SEQUENCE LISTING (22 sequences)

SEQ ID NO: 1 - protein, 487 aa, Artificial Sequence
  Anti-BCMA 4-1BB CAR amino acid sequence
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 714 aa, Artificial Sequence
  Anti-BCMA 4-1BB CAR armored with soluble IL-18 amino acid sequence
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 3 - protein, 671 aa, Artificial Sequence
  Anti-BCMA 4-1BB CAR armored with soluble IL-15 CAR amino acid sequence
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 670 aa, Artificial Sequence
  Anti-BCMA CD28 CAR armored with soluble IL-15 amino acid sequence
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 748 aa, Artificial Sequence
  Anti-BCMA 4-1BB CAR armored with membrane-bound IL-18 amino acid sequence
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDPTNGPKIPSIATGMVGALLLLLVVALGIGLFMRR

SEQ ID NO: 6 - protein, 486 aa, Artificial Sequence
  Anti-CD19 4-1BB CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 713 aa, Artificial Sequence
  Anti-CD19 4-1BB CAR armored with soluble IL-18 amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 8 - protein, 747 aa, Artificial Sequence
  Anti-CD19 4-1BB CAR armored with membrane-bound IL-18 amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDPTNGPKIPSIATGMVGALLLLLVVALGIGLFMRR

SEQ ID NO: 9 - protein, 487 aa, Artificial Sequence
  Anti-GPC3 4-1BB CAR amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 714 aa, Artificial Sequence
  Anti-GPC3 4-1BB CAR armored with soluble IL-18 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 11 - DNA, 2289 bp, Artificial Sequence
  Anti-BCMA 4-1BB CAR armored with 5xNFkb 5xAP- inducible IL-15 nucleic acid sequence
atggcactgccagtgacagcactgctgctgcctctggcactgctgctgcacgcagcaaggcctgccgtgcagctggtggagtccggcggcggcctggtgcaggccggcgactctctgagactgacatgcaccgcctccggcagggccttctctacatactttatggcctggttcagacaggccccaggcaaggagagggagtttgtggcaggaatcgcatggtctggaggaagcaccgcatacgcagactctgtgaagggccgcttcacaatcagccgggataacgccaagaataccgtgtatctgcagatgaactccctgaagtctgaggataccgccgtgtactattgcgcctccagaggcatcgaggtggaggagtttggagcatggggacagggaacacaggtgaccgtgagctccggaggaggaggatctcaggtgcagctggaggagtccggaggaggatctgtgcaggcaggaggcagcctgaggctgtcctgtgcctacacatatagcacctactccaactactatatgggatggtttagggaggcaccaggcaaggcccggacatctgtggccatcatctctagcgacaccacaatcacctacaaggatgccgtgaagggcagattcacaatcagcaaggacaacgccaagaataccctgtatctgcagatgaatagcctgaagcctgaggactccgccatgtacaggtgcgccgcctggacatctgattggagcgtggcctattggggccagggcacacaggtgaccgtgtcctctaccagcaccacaacccctgcaccaaggccacctacaccagcacctaccatcgcctctcagcctctgagcctgagaccagaggcctgtaggccagcagcaggaggagcagtgcacacccggggcctggacttcgcctgcgatatctacatctgggcaccactggcaggaacatgtggagtgctgctgctgagcctggtcatcaccctgtactgcaagagaggcaggaagaagctgctgtatatctttaagcagccattcatgcgccccgtgcagacaacccaggaggaggacggctgctcctgtcggtttccagaagaggaggagggaggatgtgagctgagggtgaagttcagcaggtccgcagatgcaccagcataccagcagggccagaatcagctgtataacgagctgaatctgggccggagagaggagtatgacgtgctggataagaggaggggaagggatccagagatgggaggcaagcctcggagaaagaacccacaggagggcctgtacaatgagctgcagaaggacaagatggccgaggcctatagcgagatcggcatgaagggagagaggaggaggggcaagggacacgatggcctgtaccagggcctgtccacagccaccaaggacacctatgatgccctgcacatgcaggccctgccaccccggtaactcacacaaaaaaccaacacacagatgtaatgaaaataaagatattttattttaggatgtattgatgaacatctgcacgatgtgcacaaagctctgcaggaactccttgatgttcttctcctccagctcctcacactccttgcagccgctctctgtcacattgccgttggagctcagagaattgttggccaggatgatcagattctccacggtatcgtggatagaggcgtcgccagactccagggagatgacctgcagctccagcagaaaacacttcatggcggtcaccttgcaggaagggtgcacatcgctctctgtatacagggtggcgtcgatgtgcatagactggatcagatcctcgatcttcttcaggtcggagatcacattcacccagttggcctctgtcttgggcaggccggcgctaaagcagcccaggatgaacacgtggatgccggcctcggtcaggaagtgagagttcagcagcagacacaggtagcactggatgctgatagatctcaggtggggcttggagatccgcatggctctgtctcaggtcagtatagaagctttgatgtgaagtcagccaagaacagctgaacactacttctgctgaggcccttttataggagggattgcttcctgtgaataataggaggatattgtccacatccagtaaagaggaaatccccaactgcatccaaaaagttttctgggaatatccactgctgcaggtgactcactgagtcagtgactcaagtggaaagtccccagtggaaagtccccagtggaaagtccccagtggaaagtccccagtggaaagtcccc

SEQ ID NO: 12 - DNA, 2261 bp, Artificial Sequence
  Anti-BCMA 4-1BB CAR armored with 3xNFkb 3xAP- inducible IL-15 nucleic acid sequence
atggcactgccagtgacagcactgctgctgcctctggcactgctgctgcacgcagcaaggcctgccgtgcagctggtggagtccggcggcggcctggtgcaggccggcgactctctgagactgacatgcaccgcctccggcagggccttctctacatactttatggcctggttcagacaggccccaggcaaggagagggagtttgtggcaggaatcgcatggtctggaggaagcaccgcatacgcagactctgtgaagggccgcttcacaatcagccgggataacgccaagaataccgtgtatctgcagatgaactccctgaagtctgaggataccgccgtgtactattgcgcctccagaggcatcgaggtggaggagtttggagcatggggacagggaacacaggtgaccgtgagctccggaggaggaggatctcaggtgcagctggaggagtccggaggaggatctgtgcaggcaggaggcagcctgaggctgtcctgtgcctacacatatagcacctactccaactactatatgggatggtttagggaggcaccaggcaaggcccggacatctgtggccatcatctctagcgacaccacaatcacctacaaggatgccgtgaagggcagattcacaatcagcaaggacaacgccaagaataccctgtatctgcagatgaatagcctgaagcctgaggactccgccatgtacaggtgcgccgcctggacatctgattggagcgtggcctattggggccagggcacacaggtgaccgtgtcctctaccagcaccacaacccctgcaccaaggccacctacaccagcacctaccatcgcctctcagcctctgagcctgagaccagaggcctgtaggccagcagcaggaggagcagtgcacacccggggcctggacttcgcctgcgatatctacatctgggcaccactggcaggaacatgtggagtgctgctgctgagcctggtcatcaccctgtactgcaagagaggcaggaagaagctgctgtatatctttaagcagccattcatgcgccccgtgcagacaacccaggaggaggacggctgctcctgtcggtttccagaagaggaggagggaggatgtgagctgagggtgaagttcagcaggtccgcagatgcaccagcataccagcagggccagaatcagctgtataacgagctgaatctgggccggagagaggagtatgacgtgctggataagaggaggggaagggatccagagatgggaggcaagcctcggagaaagaacccacaggagggcctgtacaatgagctgcagaaggacaagatggccgaggcctatagcgagatcggcatgaagggagagaggaggaggggcaagggacacgatggcctgtaccagggcctgtccacagccaccaaggacacctatgatgccctgcacatgcaggccctgccaccccggtaactcacacaaaaaaccaacacacagatgtaatgaaaataaagatattttattttaggatgtattgatgaacatctgcacgatgtgcacaaagctctgcaggaactccttgatgttcttctcctccagctcctcacactccttgcagccgctctctgtcacattgccgttggagctcagagaattgttggccaggatgatcagattctccacggtatcgtggatagaggcgtcgccagactccagggagatgacctgcagctccagcagaaaacacttcatggcggtcaccttgcaggaagggtgcacatcgctctctgtatacagggtggcgtcgatgtgcatagactggatcagatcctcgatcttcttcaggtcggagatcacattcacccagttggcctctgtcttgggcaggccggcgctaaagcagcccaggatgaacacgtggatgccggcctcggtcaggaagtgagagttcagcagcagacacaggtagcactggatgctgatagatctcaggtggggcttggagatccgcatggctctgtctcaggtcagtatagaagctttgatgtgaagtcagccaagaacagctgaacactacttctgctgaggcccttttataggagggattgcttcctgtgaataataggaggatattgtccacatccagtaaagaggaaatccccaactgcatccaaaaagttttctgggaatatccactgctgcaggtgactcactgagtcagtgactcaagtggaaagtccccagtggaaagtccccagtggaaagtcccc

SEQ ID NO: 13 - DNA, 1461 bp, Artificial Sequence
  Anti-BCMA 4-1BB CAR nucleic acid sequence
atggccctgcccgtgaccgccctgctgctgcccctggccctgctgctgcacgccgccaggcccgccgtgcagctggtggagagcggcggcggcctggtgcaggccggcgacagcctgaggctgacctgcaccgccagcggcagggccttcagcacctacttcatggcctggttcaggcaggcccccggcaaggagagggagttcgtggccggcatcgcctggagcggcggcagcaccgcctacgccgacagcgtgaagggcaggttcaccatcagcagggacaacgccaagaacaccgtgtacctgcagatgaacagcctgaagagcgaggacaccgccgtgtactactgcgccagcaggggcatcgaggtggaggagttcggcgcctggggccagggcacccaggtgaccgtgagcagcggcggcggcggcagccaggtgcagctggaggagagcggcggcggcagcgtgcaggccggcggcagcctgaggctgagctgcgcctacacctacagcacctacagcaactactacatgggctggttcagggaggcccccggcaaggccaggaccagcgtggccatcatcagcagcgacaccaccatcacctacaaggacgccgtgaagggcaggttcaccatcagcaaggacaacgccaagaacaccctgtacctgcagatgaacagcctgaagcccgaggacagcgccatgtacaggtgcgccgcctggaccagcgactggagcgtggcctactggggccagggcacccaggtgaccgtgagcagcaccagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 14 - DNA, 2142 bp, Artificial Sequence
  Anti-BCMA 4-1BB CAR armored with soluble IL-18 nucleic acid sequence
atggccctgcccgtgaccgccctgctgctgcccctggccctgctgctgcacgccgccaggcccgccgtgcagctggtggagagcggcggcggcctggtgcaggccggcgacagcctgaggctgacctgcaccgccagcggcagggccttcagcacctacttcatggcctggttcaggcaggcccccggcaaggagagggagttcgtggccggcatcgcctggagcggcggcagcaccgcctacgccgacagcgtgaagggcaggttcaccatcagcagggacaacgccaagaacaccgtgtacctgcagatgaacagcctgaagagcgaggacaccgccgtgtactactgcgccagcaggggcatcgaggtggaggagttcggcgcctggggccagggcacccaggtgaccgtgagcagcggcggcggcggcagccaggtgcagctggaggagagcggcggcggcagcgtgcaggccggcggcagcctgaggctgagctgcgcctacacctacagcacctacagcaactactacatgggctggttcagggaggcccccggcaaggccaggaccagcgtggccatcatcagcagcgacaccaccatcacctacaaggacgccgtgaagggcaggttcaccatcagcaaggacaacgccaagaacaccctgtacctgcagatgaacagcctgaagcccgaggacagcgccatgtacaggtgcgccgcctggaccagcgactggagcgtggcctactggggccagggcacccaggtgaccgtgagcagcaccagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatgaggatcagcaagccccacctgaggagcatcagcatccagtgctacctgtgcctgctgctgaacagccacttcctgaccgaggccggcatccacgtgttcatcctgggctgcttcagcgccggcctgcccaagaccgaggcctacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggac

SEQ ID NO: 15 - DNA, 2013 bp, Artificial Sequence
  Anti-BCMA 4-1BB CAR armored with soluble IL-15 CAR nucleic acid sequence
atggcactgcctgtcacggcccttctgctcccgctggctctgctcctgcacgccgcacgtccagcggtgcagttggtggagagcggaggtggcctcgtgcaggccggcgattctttgcggctgacctgtacagcatcgggccgtgcgttctctacctatttcatggcatggttccgccaggcgcctggcaaagagcgcgagttcgttgctggcatagcctggtctggaggcagtaccgcttacgcggacagcgtgaagggccggttcaccatctctcgcgacaacgccaagaacaccgtgtacctgcagatgaactccctcaagtcggaggacaccgctgtctactactgcgcctccaggggcatcgaggtagaggagttcggtgcttggggccaaggcacccaggtgacggtctcctccggcggtggaggtagccaggtccagctggaggagagtggcggcggctccgtgcaggccggcggttcgctgcgcctgtcctgtgcctacacctactccacgtactcaaactactacatgggctggttccgggaggccccaggcaaggcccgcacctccgtggccatcatcagctccgacaccaccatcacttacaaggacgccgtgaaaggtcgtttcaccatctccaaggacaacgcgaagaacaccctgtacctgcagatgaattccctgaagcccgaagactcggctatgtataggtgtgctgcttggaccagcgattggtctgtggcttattggggccagggcacccaggtcacagtgagctctacatcaactacaacccccgccccgcgccccccaaccccggctccgactatcgcttcccagccattgtctctccgccctgaagcttgtagacctgcagccggcggcgccgtccatactcgcggtttggacttcgcctgcgacatctatatctgggcgcccctggccggtacctgcggggtgctgctgctgagtctggtcatcaccctttactgtaagcgtggccgcaagaagctgttgtacatcttcaagcagcccttcatgcgtccggtgcagacgacccaggaggaagacggatgctcttgccgattccctgaggaagaggagggcgggtgtgaactcagagtaaaatttagccgctcggctgacgcacccgcctaccagcagggacagaaccagctgtacaacgagctcaacctgggccgccgcgaagagtacgatgttttggataaacgccgcggtcgagacccggagatgggaggtaagcccaggcgcaaaaaccctcaggagggcctgtacaacgagctacagaaagacaagatggccgaggcgtattccgagatcggtatgaagggcgagcggcgcagagggaaaggccacgacggcctttatcagggcctctccactgccaccaaggatacttacgacgcacttcacatgcaggccctgcccccgcgtgggagcggggctaccaactttagcctgctgaagcaggcgggagatgtggaggagaatccagggcccatgcgcatctctaaacctcatttgcgctcgatctcgattcagtgctacctgtgcctgctactcaactcccactttctgaccgaagcaggcatccatgttttcatcttagggtgctttagcgccgggctacccaagactgaggccaactgggtcaacgtgatttccgaccttaagaagattgaggacctgatccagtcgatgcacattgacgccactctgtacacggagtccgatgtgcaccccagctgtaaggtgacggctatgaagtgctttctgctggaattgcaggtgatttccctggagtctggagacgcgtcaatccacgacacggtagagaacctgatcatcctggcgaacaactccctctcgagcaatggcaacgtgactgagagcgggtgtaaggagtgcgaggagctcgaggagaagaatatcaaggagttcctgcaatccttcgtccacatcgtgcagatgtttattaatactagc

SEQ ID NO: 16 - DNA, 2010 bp, Artificial Sequence
  Anti-BCMA CD28 CAR armored with soluble IL-15 nucleic acid sequence
atggctttgccggtgaccgctctgctgctgcccctggctttgctgcttcacgccgctcgccctgccgtgcaactcgtggaatctggcggcggactggtccaggcgggtgattctctccggttgacatgcactgcttccgggagggcgttctccacctatttcatggcgtggttccgccaggcgccgggcaaggaacgcgagttcgtggcgggcatcgcgtggtctgggggttcgactgcctacgcggacagtgtcaagggacggttcaccatcagccgcgacaacgcgaagaacacggtatacctgcagatgaatagcctgaaatccgaagatactgcagtgtattactgtgcctcccgcggtatcgaggtggaggagttcggcgcctggggccagggcacccaggtcaccgtgtcgtccggcggcggtggctcccaagtgcagttggaagagagcggcgggggctccgtacaggctgggggctcccttcgcctgagctgcgcctacacctactctacctacagcaactactacatgggttggttcagagaggctcccgggaaggcgcgcacttccgtggccatcatctcttccgacacgactatcacctacaaggacgctgtgaagggaagattcacgatctcaaaagacaatgccaagaacactctctacctccagatgaactccctgaagcctgaagacagcgcaatgtataggtgtgccgcttggacgagcgattggtctgtcgcatattggggccaggggacccaggtgacagtgtcctcgacgagcaccaccacacctgctcctaggcccccaactccggcgcccaccattgcttcacagccactgtctctgcgcccggaggcctgccgaccggccgctggaggcgctgtgcatacacgtggtttggatttcgcctgtgacatctacatctgggcccccctggccgggacctgcggggtgctgctgctttcgctggtgatcaccctatactgtcgctccaagcgcagtcgcctacttcacagtgattacatgaacatgactccccgccgtcccggccctacccgcaagcactaccagccctatgcccccccgcgtgacttcgctgcttaccggagccgcgtcaaattttcacgcagtgcggacgcgcctgcctatcagcagggacagaaccagctttacaacgagctcaacctgggccggcgcgaggagtacgacgtgctggacaagcgccgtggacgtgatccggagatgggcggaaaacctcggcgcaaaaatcctcaggagggcctttacaacgagcttcagaaggacaaaatggccgaggcttactcggagatcggtatgaagggcgagcgccgtcgcggcaaagggcacgacggcctgtaccagggattatcgactgctaccaaggatacatacgacgcgctccacatgcaggccctgcctccccgtggctccggtgcaaccaacttctccctcctcaagcaggccggtgacgtggaggagaatccaggccccatgcgcatctccaagccgcacctgaggtccatttccatacaatgttacctgtgcctgttgctcaacagccactttctgaccgaggccggcatccacgtgttcatcctgggttgcttttcggccggcctgccgaagaccgaggctaactgggttaacgtgatctctgatctaaagaagattgaggacctgatccagtccatgcatattgacgccaccctgtacacggagagtgacgtgcacccctcttgtaaggtgaccgccatgaagtgctttctgctggagctgcaggtcatcagcttggagtctggggacgcatccattcatgacaccgtggagaacctgattatcctggccaacaactctctgtcctcaaatggcaacgtcaccgagagcggctgtaaggaatgcgaggagctggaggagaagaacatcaaggagttcctgcagtccttcgtccacatcgtccagatgtttattaacacgtct

SEQ ID NO: 17 - DNA, 2244 bp, Artificial Sequence
  Anti-BCMA 4-1BB CAR armored with membrane-bound IL-18 nucleic acid sequence
atggccctgcccgtgaccgccctgctgctgcccctggccctgctgctgcacgccgccaggcccgccgtgcagctggtggagagcggcggcggcctggtgcaggccggcgacagcctgaggctgacctgcaccgccagcggcagggccttcagcacctacttcatggcctggttcaggcaggcccccggcaaggagagggagttcgtggccggcatcgcctggagcggcggcagcaccgcctacgccgacagcgtgaagggcaggttcaccatcagcagggacaacgccaagaacaccgtgtacctgcagatgaacagcctgaagagcgaggacaccgccgtgtactactgcgccagcaggggcatcgaggtggaggagttcggcgcctggggccagggcacccaggtgaccgtgagcagcggcggcggcggcagccaggtgcagctggaggagagcggcggcggcagcgtgcaggccggcggcagcctgaggctgagctgcgcctacacctacagcacctacagcaactactacatgggctggttcagggaggcccccggcaaggccaggaccagcgtggccatcatcagcagcgacaccaccatcacctacaaggacgccgtgaagggcaggttcaccatcagcaaggacaacgccaagaacaccctgtacctgcagatgaacagcctgaagcccgaggacagcgccatgtacaggtgcgccgcctggaccagcgactggagcgtggcctactggggccagggcacccaggtgaccgtgagcagcaccagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatgaggatcagcaagccccacctgaggagcatcagcatccagtgctacctgtgcctgctgctgaacagccacttcctgaccgaggccggcatccacgtgttcatcctgggctgcttcagcgccggcctgcccaagaccgaggcctacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggaccccaccaacggccccaagatccccagcatcgccaccggcatggtgggcgccctgctgctgctgctggtggtggccctgggcatcggcctgttcatgaggagg

SEQ ID NO: 18 - DNA, 1458 bp, Artificial Sequence
  Anti-CD19 4-1BB CAR nucleic acid sequence
atggccctgcccgtgaccgccctgctgctgcccctggccctgctgctgcacgccgccaggcccgacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 19 - DNA, 2139 bp, Artificial Sequence
  Anti-CD19 4-1BB CAR armored with soluble IL-18 nucleic acid sequence
atggccctgcccgtgaccgccctgctgctgcccctggccctgctgctgcacgccgccaggcccgacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatgaggatcagcaagccccacctgaggagcatcagcatccagtgctacctgtgcctgctgctgaacagccacttcctgaccgaggccggcatccacgtgttcatcctgggctgcttcagcgccggcctgcccaagaccgaggcctacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggac

SEQ ID NO: 20 - DNA, 2241 bp, Artificial Sequence
  Anti-CD19 4-1BB CAR armored with membrane-bound IL-18 nucleic acid sequence
atggccctgcccgtgaccgccctgctgctgcccctggccctgctgctgcacgccgccaggcccgacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatgaggatcagcaagccccacctgaggagcatcagcatccagtgctacctgtgcctgctgctgaacagccacttcctgaccgaggccggcatccacgtgttcatcctgggctgcttcagcgccggcctgcccaagaccgaggcctacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggaccccaccaacggccccaagatccccagcatcgccaccggcatggtgggcgccctgctgctgctgctggtggtggccctgggcatcggcctgttcatgaggagg

SEQ ID NO: 21 - DNA, 1461 bp, Artificial Sequence
  Anti-GPC3 4-1BB CAR nucleic acid sequence
atggccctgcccgtgaccgccctgctgctgcccctggccctgctgctgcacgccgccaggcccgacgtggtgatgacccagagccccctgagcctgcccgtgacccccggcgagcccgccagcatcagctgcaggagcagccagagcctggtgcacagcaacgccaacacctacctgcactggtacctgcagaagcccggccagagcccccagctgctgatctacaaggtgagcaacaggttcagcggcgtgcccgacaggttcagcggcagcggcagcggcaccgacttcaccctgaagatcagcagggtggaggccgaggacgtgggcgtgtactactgcagccagaacacccacgtgccccccaccttcggccagggcaccaagctggagatcaagaggggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgactacgagatgcactgggtgaggcaggcccccggccagggcctggagtggatgggcgccctggaccccaagaccggcgacaccgcctacagccagaagttcaagggcagggtgaccctgaccgccgacgagagcaccagcaccgcctacatggagctgagcagcctgaggagcgaggacaccgccgtgtactactgcaccaggttctacagctacacctactggggccagggcaccctggtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 22 - DNA, 2142 bp, Artificial Sequence
  Anti-GPC3 4-1BB CAR armored with soluble IL-18 nucleic acid sequence
atggccctgcccgtgaccgccctgctgctgcccctggccctgctgctgcacgccgccaggcccgacgtggtgatgacccagagccccctgagcctgcccgtgacccccggcgagcccgccagcatcagctgcaggagcagccagagcctggtgcacagcaacgccaacacctacctgcactggtacctgcagaagcccggccagagcccccagctgctgatctacaaggtgagcaacaggttcagcggcgtgcccgacaggttcagcggcagcggcagcggcaccgacttcaccctgaagatcagcagggtggaggccgaggacgtgggcgtgtactactgcagccagaacacccacgtgccccccaccttcggccagggcaccaagctggagatcaagaggggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgactacgagatgcactgggtgaggcaggcccccggccagggcctggagtggatgggcgccctggaccccaagaccggcgacaccgcctacagccagaagttcaagggcagggtgaccctgaccgccgacgagagcaccagcaccgcctacatggagctgagcagcctgaggagcgaggacaccgccgtgtactactgcaccaggttctacagctacacctactggggccagggcaccctggtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatgaggatcagcaagccccacctgaggagcatcagcatccagtgctacctgtgcctgctgctgaacagccacttcctgaccgaggccggcatccacgtgttcatcctgggctgcttcagcgccggcctgcccaagaccgaggcctacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggac

(19) United States
(12) Patent Application Publication
(10) Pub. No.: US 2023/0226181 A1
(43) Pub. Date: Jul. 20, 2023

(54) GENETIC ENGINEERING OF gamma delta T CELLS FOR IMMUNOTHERAPY

(71) Applicant: Nanjing Legend Biotech Co., Ltd., Nanjing (CN)

(72) Inventors: Zhongyuan Tu, Nanjing (CN); Yafeng Zhang, Nanjing (CN); Shu Wu, Nanjing (CN); Yi Xu, Nanjing (CN)

(21) Appl. No.: 18/011,766

(22) PCT Filed: Jun. 22, 2021
(86) PCT No.: PCT/CN2021/101462

§ 371 Date: Dec. 20, 2022

(30) Foreign Application Priority Data
Jun. 22, 2020 (WO) PCT/CN2020/097439

(51) Int. Cl. A61K 39/00; C07K 14/725; C07K 14/715; C07K 16/28; A61P 35/00; C07K 14/705; C07K 16/30; C07K 14/54

(52) U.S. Cl. CPC A61K 39/4611; C07K 14/7051; C07K 14/7155; C07K 16/2803; C07K 16/2878; A61P 35/00; C07K 14/70578; C07K 16/303; A61K 39/464474; A61K 39/464412; C07K 14/70521; C07K 14/54; A61K 39/464417; A61K 39/4631; A61K 2239/28

(57) ABSTRACT

The present disclosure relates to a novel platform for immunotherapy which combines CAR engineered γδ T cells with armoring interleukin IL-18 that can be expressed constitutively or inducibly, or with a chimeric cytokine receptor comprising the endodomain of the IL-18 receptor. The system/platform and the associated methods according to the present disclosure have advantages such as increased immune cell potency and persistence for therapeutic applications.

This application claims priority benefits of International Patent Application No. PCT/CN2020/097439 filed Jun. 22, 2020, the contents of which are incorporated herein by reference in their entirety.

### RELATED APPLICATIONS AND INCORPORATION BY REFERENCE

All the patent or patent applications cited or referenced herein, and all documents cited therein or during their prosecution (“appln cited documents”) and all documents cited or referenced in the appln cited documents, and all the other documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The contents of the following submission on ASCII text file are incorporated herein by reference in their entirety: a computer readable form (CRF) of the Sequence Listing (file name: P10828-PCT.210617. Sequence listing_ST25.txt, date recorded: Jun. 22, 2021, size: 88 KB).

### FIELD OF THE INVENTION

The present disclosure relates to genetically engineered immunoresponsive cells for therapeutic and related applications. In particular, the present disclosure relates to armored CAR γδ T cells.

### BACKGROUND OF THE INVENTION

Immunotherapy with chimeric antigen receptor (CAR) T cells offers a promising method to improve cure rates and decrease morbidities for patients with cancer. In this regard, CD19-specific CAR T cell therapies have achieved dramatic objective responses for a high percent of patients with CD19-positive leukemia or lymphoma (1-2). Most patients with other hematologic tumor or solid tumors however, have experienced transient or no benefit from CAR T cell therapies (3-5). Novel strategies are therefore needed to improve CAR T cell function for patients with these tumors. One of the obstacles for the field is limited CAR T cell persistence after infusion into patients. Another obstacle is hostile tumor microenvironment suppresses CAR T cell function.

T cells can be subdivided into conventional and unconventional T cells, based on their function and expression of TCRs and coreceptors (6). Conventional T cells express the αβ variant of the TCR together with either the CD4 or CD8αβ coreceptor, they belong to adaptive immune cells. The unconventional T cells on the other hand have been postulated to express either the αβ TCR or the γδ TCR. The cells expressing γδ TCR are γδ T cells. They have both adaptive and innate characteristics.

Besides CAR-αβ T cell therapy, people also use γδ T cells for cell therapy, especially for allogenic cell therapy (7). The advantage of γδ T cells for allogeneic applications is that the γδ TCR binds to ligand in a MHC independent manner, so γδ T cells are not alloreactive and don't cause GvHD (graft-versus-host disease). Besides, γδ T cells are more like innate cells on tumor killing or pathogen clearance. They can respond and kill tumor or infected cells rapidly but release less cytokines for proliferation. On the other hand, however, the persistence of large numbers in vivo is often limited to a few days. Therefore, both CAR expression and CAR engineered cell expansion and persistence are critical for proper CAR functionality, which continuously needs new approach and improvements.

Citation or identification of any document in this application is not an admission that such document is available as prior art to the present invention.

### SUMMARY OF THE INVENTION

The present invention provides a novel platform which modified CAR (or TCR) engineered γδ T cells with an interleukin IL-18 armor. The CAR (or TCR) and IL-18 can be transcribed from one nucleic acid or two separate nucleic acids. The expression of IL-18 can be constitutive or inducible to meet different needs. Other than expressing an exogenous IL-18 polypeptide or a variant thereof, the armoring effect can also be achieved by using a chimeric cytokine receptor comprising the endodomain of the IL-18 receptor (IL-18R, IL-18R(a and/or IL-18Rβ) and the exodomain of another cytokine or an artificial ligand. Along with other advantages, the resultant platform, i.e. IL-18 armored CAR (or TCR) engineered γδ T cells, has an improved T cell expansion and persistence, as well as increased tumor-killing potency.

In an aspect of the present disclosure, there is provided an engineered γδ T cell comprising:
(i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

In certain embodiments, the IL-18 receptor comprises IL-18Ra, IL-18RP, or the combination thereof.

In certain embodiments, the chimeric cytokine receptor further comprises the exodomain of a cytokine other than IL-18, or an artificial ligand.

In certain embodiments, the IL-18 is in soluble form (sIL-18) or membrane-bound form (mbIL-18).

In certain embodiments, the engineered γδ T cell is selected from the group consisting of γδ T cell, 81 T cell, 63 T cell, or the combination thereof.

In certain embodiments, the first nucleic acid further comprises a first regulatory region which comprises a promoter operatively linked to the first nucleic acid sequence.

In certain embodiments, the second nucleic acid sequence further comprises a second regulatory region operatively linked to the second nucleic acid sequence.

In certain embodiments, the second regulatory region comprises (i) an inducible promoter, and/or (ii) a promoter and one or more transcription factor binding sites, wherein the transcription factor binding sites bind to transcription factors that are active in activated γδ T cells.

In certain embodiments, the transcription factor binding sites comprise one or more copies of the transcription factor binding site selected from the group consisting of NF-κB, AP-1, Myc, NR4A, TOX1, TOX2, TOX3, TOX4, STAT1, STAT2, STAT3, STAT4, STAT5, STAT6, or combinations thereof.

In certain embodiments, the promoter comprises an IFN-β promoter, an IL-2 promoter, a BCL-2 promoter, a GM-CSF promoter, an IL-6 promoter, an IFN-γ promoter, an IL-12 promoter, an IL-4 promoter, an IL-15 promoter, an IL-18 promoter or an IL-21 promoter.

In certain embodiments, the first nucleic acid and the second nucleic acid are comprised in one vector. In certain embodiments, the first nucleic acid and the second nucleic acid are under control of one promoter.

In certain embodiments, the first nucleic acid and the second nucleic acid are under control of two promoters. In certain embodiments, the first nucleic acid and the second nucleic acid are transcribed in opposite directions.

In certain embodiments, the first nucleic acid and the second nucleic acid are comprised in separate vectors.

In certain embodiments, the vector is a virus vector.

In certain embodiments, the virus vector is a lentivirus vector, retrovirus vector, adenoviral vectors, adeno-associated virus vectors, vaccinia vector, or herpes simplex viral vector.

In certain embodiments, the extracellular antigen recognition domain is selective for a tumor antigen or an infectious disease-associated antigen.

In certain embodiments, the tumor antigen is selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof.

In certain embodiments, the extracellular antigen recognition domain is monospecific. In certain embodiments, the CAR is single CAR. In certain embodiments, the tumor antigen comprises BCMA, GPC3 and CD19.

In certain embodiments, the extracellular antigen recognition domain is multispecific.

In certain embodiments, the CAR is a tandem CAR or dual CAR. In certain embodiments, the tandem CAR or dual CAR targets the same tumor antigen. In certain embodiments, the tandem CAR or dual CAR targets different epitopes on the same tumor antigen. In certain embodiments, the tandem CAR or dual CAR targets different tumor antigens. In certain embodiments, the tumor antigen comprises BCMA, GPC3 and/or CD19.

In certain embodiments, the tandem CAR comprises: more than one antigen-binding portions that target different epitopes of BCMA, a transmembrane domain, and an intracellular signaling domain.

In certain embodiments, the tandem CD19 comprises: more than one antigen-binding portions that target different epitopes of CD19, a transmembrane domain, and an intracellular signaling domain.

In certain embodiments, the tandem GPC3 comprises: more than one antigen-binding portions that target different epitopes of GPC3, a transmembrane domain, and an intracellular signaling domain.

In certain embodiments, the intracellular signaling domain comprises a primary intracellular signaling domain of an immune effector cell derived from a signal transducing molecule selected from the group consisting of CD3ζ, FcRγ, FcRβ, CD3γ, CD3δ, CD3ε, CD5, CD22, CD79a, CD79b, CD66d and combinations thereof.

In certain embodiments, the intracellular signaling domain comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.

In certain embodiments, the transmembrane domain is from CD4, CD8U, CD28, or ICOS.

In certain embodiments, the nucleic acid sequence that encodes a CAR further comprises a hinge region located between the extracellular antigen recognition domain and the transmembrane domain.

In certain embodiments, both the first nucleic acid and the second nucleic acid have a leading peptide.

In certain embodiments, the engineered γδ T cell comprises a nucleic acid having a nucleotide sequence at least about 80%, 85%, 90%, 91%, 92%, 93%, 94%, 95%, 96%, 97%, 98%, 99% or 100% identical to SEQ ID NO: 14, 17, 19, 20 or 22. In certain embodiments, the engineered γδ T cell comprises a nucleic acid of any one of SEQ ID NO: 14, 17, 19, 20 or 22. In certain embodiments, the engineered γδ T cell comprises a nucleic acid of SEQ ID NO: 14. In certain embodiments, the engineered γδ T cell comprises a nucleic acid of SEQ ID NO: 17. In certain embodiments, the engineered γδ T cell comprises a nucleic acid of SEQ ID NO: 19. In certain embodiments, the engineered γδ T cell comprises a nucleic acid of SEQ ID NO: 20. In certain embodiments, the engineered γδ T cell comprises a nucleic acid of SEQ ID NO: 22.

In certain embodiments, wherein the engineered γδ T cell is allogeneic. In certain embodiments, the engineered γδ T cell is autologous.

In an aspect of the present disclosure, there is provided an engineered γδ T cell comprising:
(i) a first nucleic acid, which comprises a first regulatory region operatively linked to a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor,(b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R),

wherein the extracellular antigen recognition domain is selective for a tumor antigen selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof;

the intracellular signaling domain comprises a primary intracellular signaling domain of an immune effector cell derived from a signal transducing molecule selected from the group consisting of CD3ζ, FcRγ, FcRβ, CD3γ, CD3δ, CD3ε, CD5, CD22, CD79a, CD79b, CD66d and combinations thereof, and the intracellular signaling domain further comprises an intracellular co-stimulatory domain derived from a co-stimulatory molecule selected from the group consisting of CD27, CD28, 4-IBB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSFI4, HAVCR1, LGALS9, DAP10, DAP12, CD83, ligands of CD83 and combinations thereof;

the transmembrane domain is from CD4, CD8a, CD28, or ICOS; and

optionally, the second nucleic acid sequence further comprises a second regulatory region which is inducible and operatively linked to the second nucleic acid sequence.

In an aspect of the present disclosure, there is provided an engineered γδ T cell comprising:
(i) a first nucleic acid, which comprises a first regulatory region operatively linked to a first nucleic acid sequence that encodes a chimeric antigen receptor (CAR) comprising: more than one tandem antigen recognition portions targeting BCMA; a transmembrane domain selected from CD4, CD8a, CD28, or ICOS; a CD3ζ intracellular signaling domain; and a CD28 or 4-1BB intracellular co-stimulatory domain;
and
(ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R).

In an aspect, there is provided an engineered γδ T cell comprising a nucleic acid that comprises from N-terminus to C-terminus: a promoter, a leading peptide, an anti-BCMA extracellular antigen recognition domain comprising more than one tandem antigen binding portions, a transmembrane domain, a CD28 or 4-1BB intracellular co-stimulatory domain, a CD3(intracellular signaling domain, a P2A self-cleaving peptide, a leading peptide, and a IL-18 encoding sequence.

In an aspect, there is provided an engineered γδ T cell comprising a nucleic acid that comprises from N-terminus to C-terminus: a promoter, a leading peptide, an anti-BCMA extracellular antigen recognition domain comprising more than one tandem antigen binding portions, a transmembrane domain, a CD28 or 4-1BB intracellular co-stimulatory domain, a CD3(intracellular signaling domain, a PA2 polyadenylation site, a IL-18 encoding sequence, a leading peptide, and a promoter and NF-κB and/or AP-1 inducible elements.

In an aspect of the present disclosure, there is provided an engineered γδ T cell comprising:
(i) a first nucleic acid, which comprises a first regulatory region operatively linked to a first nucleic acid sequence that encodes a chimeric antigen receptor (CAR) comprising: more than one tandem antigen recognition portions targeting CD19; a transmembrane domain selected from CD4, CD8a, CD28, or ICOS; a CD3 intracellular signaling domain; and a CD28 or 4-1BB intracellular co-stimulatory domain;
and
(ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R).

In an aspect, there is provided an engineered γδ T cell comprising a nucleic acid that comprises from N-terminus to C-terminus: a promoter, a leading peptide, an anti-CD19 extracellular antigen recognition domain comprising more than one tandem antigen binding portions, a transmembrane domain, a CD28 or 4-1BB intracellular co-stimulatory domain, a CD3(intracellular signaling domain, a P2A self-cleaving peptide, a leading peptide, and a IL-18 encoding sequence.

In an aspect, there is provided an engineered γδ T cell comprising a nucleic acid that comprises from N-terminus to C-terminus: a promoter, a leading peptide, an anti-CD19 extracellular antigen recognition domain comprising more than one tandem antigen binding portions, a transmembrane domain, a CD28 or 4-1BB intracellular co-stimulatory domain, a CD3(intracellular signaling domain, a PA2 polyadenylation site, a IL-18 encoding sequence, a leading peptide, and a promoter and NF-κB and/or AP-1 inducible elements.

In an aspect of the present disclosure, there is provided an engineered γδ T cell comprising:
(i) a first nucleic acid, which comprises a first regulatory region operatively linked to a first nucleic acid sequence that encodes a chimeric antigen receptor (CAR) comprising: more than one tandem antigen recognition portions targeting GPC3; a transmembrane domain selected from CD4, CD8a, CD28, or ICOS; a CD3(intracellular signaling domain; and a CD28 or 4-1BB intracellular co-stimulatory domain;
and
(ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R).

In an aspect, there is provided an engineered γδ T cell comprising a nucleic acid that comprises from N-terminus to C-terminus: a promoter, a leading peptide, an anti-GPC3 extracellular antigen recognition domain comprising more than one tandem antigen binding portions, a transmembrane domain, a CD28 or 4-1BB intracellular co-stimulatory domain, a CD3(intracellular signaling domain, a P2A self-cleaving peptide, a leading peptide, and a IL-18 encoding sequence.

In an aspect, there is provided an engineered γδ T cell comprising a nucleic acid that comprises from N-terminus to C-terminus: a promoter, a leading peptide, an anti-GPC3 extracellular antigen recognition domain comprising more than one tandem antigen binding portions, a transmembrane domain, a CD28 or 4-1BB intracellular co-stimulatory domain, a CD3(intracellular signaling domain, a PA2 polyadenylation site, a IL-18 encoding sequence, a leading peptide, and a promoter and NF-κB and/or AP-1 inducible elements.

In an aspect, there is provided an engineered γδ T cell comprising:
(i) a chimeric antigen receptor (CAR) comprising an extracellular antigen recognition domain that is selective for a target, a transmembrane domain, and an intracellular signaling domain, and/or
a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) exogenous cytokine IL-18 or a functional variant thereof, or a chimeric cytokine receptor comprising the endodomain of the IL-18 receptor (IL-18R).

In some embodiments, the extracellular antigen recognition domain is selective for a tumor antigen selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof;

the intracellular signaling domain comprises a primary intracellular signaling domain of an immune effector cell derived from a signal transducing molecule selected from the group consisting of CD3ζ, FcRγ, FcRβ, CD3γ, CD3δ, CD3ε, CD5, CD22, CD79a, CD79b, CD66d and combinations thereof; and/or the intracellular signaling domain comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof; and the transmembrane domain is from CD4, CD8α, CD28, or ICOS.

In some embodiments, the IL-18 receptor comprises IL-18Rα, IL-18Rβ or the combination thereof. In other words, the endodomain of the chimeric cytokine receptor may comprise the endodomain of IL-18Rα, the endodomain of IL-18Rβ, or the endodomains of both IL-18Rα and IL-18Rβ.

In some embodiments, the chimeric cytokine receptor further comprises the exodomain of a cytokine other than IL-18, or an artificial ligand. In some embodiments, the IL-18 is in soluble form or membrane-bound form.

In some embodiments, the CAR is a tandem CAR targeting BCMA. In some embodiments, the CAR is a tandem CAR targeting CD19. In some embodiments, the CAR is a tandem CAR targeting GPC3.

In an aspect, there is provided an engineered γδ T cell comprising:
(i) a tandem chimeric antigen receptor (CAR) comprising more than one antigen recognition portions targeting BCMA, CD19 or GPC3, a transmembrane domain, and an intracellular signaling domain; and
(ii) an exogenous cytokine IL-18 or a functional variant thereof, or a chimeric cytokine receptor comprising the endodomain of the IL-18 receptor (IL-18R).

In some embodiments, the intracellular signaling domain is CD3ζ, the intracellular signaling domain also comprises an intracellular co-stimulatory domain CD28 or 4-1BB, and the transmembrane domain is from CD4, CD8α, CD28, or ICOS.

In some embodiments, the IL-18 receptor comprises IL-18Rα, IL-18Rβ or the combination thereof. In some embodiments, the chimeric cytokine receptor further comprises the exodomain of a cytokine other than IL-18, or an artificial ligand. In some embodiments, the IL-18 is in soluble form or membrane-bound form.

In some embodiments, the engineered γδ T cell comprises a polypeptide having an amino acid sequence at least about 80%, 85%, 90%, 91%, 92%, 93%, 94%, 95%, 96%, 97%, 98%, 99% or 100% identical to SEQ ID NO: 2, 5, 7, 8 or 10. In some embodiments, the engineered γδ T cell comprises an amino acid sequence of any one of SEQ ID NO: 2, 5, 7, 8 or 10. In some embodiments, the engineered γδ T cell comprises an amino acid sequence of SEQ ID NO: 2. In some embodiments, the engineered γδ T cell comprises an amino acid sequence of SEQ ID NO: 5. In some embodiments, the engineered γδ T cell comprises an amino acid sequence of SEQ ID NO: 7. In some embodiments, the engineered γδ T cell comprises an amino acid sequence of SEQ ID NO: 8. In some embodiments, the engineered γδ T cell comprises an amino acid sequence of SEQ ID NO: 10.

In an aspect, there is provided a pharmaceutical composition, comprising an effective amount of the engineered γδ T cell according to the present invention and a pharmaceutically acceptable excipient. In certain embodiments, the pharmaceutical composition comprises a therapeutically effective amount of the engineered γδ T cell for treating a hematological cancer or solid tumor.

In an aspect, there is provided a method of providing an anti-tumor immunity in a subject comprising administering to the subject an effective amount of the engineered γδ T cell or the pharmaceutical composition according to the invention.

In an aspect, there is provided a method of treating cancer in a subject, the method comprising administering to the subject an effective amount of the engineered γδ T cell or the pharmaceutical composition according to the invention, wherein the engineered γδ T cells treat the cancer.

In an aspect, there is provided a method of delaying or preventing metastasis or recurrence of a cancer in a subject, the method comprising administering to the subject an effective amount of the engineered γδ T cell or the pharmaceutical composition according to the invention, wherein the engineered γδ T cells delay or prevent metastasis or recurrence of the cancer.

In an aspect, there is provided a method of making a chimeric antigen receptor γδ T cell armored with IL-18, which comprises introducing into a γδ T cell:
(i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

In an aspect, there is provided a kit for making a chimeric antigen receptor γδ T cell armored with IL-18, which comprises:
(a) a container comprising
   (1) (i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
      a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
   (ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R);
   or
   (2) a vector comprising the first and second nucleic acids;
(b) a container comprising γδ T cells; and
(c) instructions for using the kit.

In an aspect, there is provided use of the engineered γδ T cell or the pharmaceutical composition according to the invention, to treat a cancer or an infectious disease in a subject.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Schematic presentation of a second generation CAR armored with soluble human IL-18. The CAR construct and cytokine are expressed on the same transcript. P2A indicates a short, virus-derived peptide sequence that mediate a ribosome-skipping event and enables generation of separate peptide products from one mRNA.

FIG. 2: Schematic presentation of antigen recognition domain fused TCR armored with soluble human IL-18.

FIG. 3: Schematic presentation of a second generation CAR armored with membrane bound human IL-18.

FIG. 4: Schematic presentation of antigen recognition domain fused TCR armored with membrane bound human IL-18.

FIG. 5: Schematic presentation of a second generation CAR armored with IL-18 based chimeric cytokine receptor.

FIG. 6: Schematic presentation of antigen recognition domain fused TCR armored with IL-18 based chimeric cytokine receptor.

FIG. 7: Second generation CAR with 4-1BB costimulatory domain armored with soluble IL-18 (sIL-18, FIG. 7A) and soluble IL-15 (sIL-15, FIG. 7B), membrane-bound IL-18 (mbIL-18, FIG. 7C) and CAR with CD28 costimulatory domain armored with soluble IL-15 (sIL-15, FIG. 7D).

FIG. 8: Second generation CAR with 4-1BB armored with soluble IL-15 under 5 NF-κB×5 AP-1 (FIG. 8A) and 3 NF-κB ×3 AP-1 inducible elements (FIG. 8B). The CAR construct and IL15 are expressed in opposite directions from their respective promoters.

FIG. 9: Cytotoxicity of CAR-γδ T cells with different molecular designs of cytokine armors on multiple myeloma tumor cell lines H929, RPMI-8226 and NCI-H929 (FIGS. 9A, 9B, 9C and 9D), B cell malignancies cell line Raji (FIG. 9E) and liver cancer cell line Huh7 (FIG. 9F)

FIG. 10: In vitro IL-15 (FIG. 10A) and IL-18 (FIG. 10B) cytokine release from γδ T cells with different molecular designs of cytokine armors.

FIG. 11: TNF-α (FIG. 11A), GM-CSF (FIG. 11B) and IFN-γ (FIG. 11C) cytokine release from γδ T cells with different molecular designs of cytokine armors.

FIG. 12: In vitro long-term cytotoxicity and persistence of anti-BCMA armored and unarmored CAR-γδ T cells (FIGS. 12A and 12B), anti-CD19 armored and unarmored CAR-γδ T cells (FIGS. 12C and 12D) and anti-GPC3 armored and unarmored CAR-γδ T cells (FIGS. 12E and 12F).

FIG. 13: In vivo efficacy of γδ T cells with different molecular designs of cytokine armors on multiple myeloma (FIGS. 13A and 13B), B cell malignancies (FIG. 13C) and liver cancer (FIG. 13D) animal models.

FIG. 14: In vivo IL-15 (FIG. 14A) and IL-18 (FIG. 14B) cytokine release from γδ T cells with different molecular designs of cytokine armors in the multiple myeloma animal model described in FIG. 13A. In vivo TNF-α (FIG. 14C), GM-CSF (FIG. 14D) and IFN-γ (FIG. 14E) cytokine release from γδ T cells with different molecular designs of cytokine armors in the multiple myeloma animal model described in FIG. 13A. In vivo IFN-γ (FIG. 14F), TNF-α (FIG. 14G) and GM-CSF (FIG. 14H) cytokine release from γδ T cells with different molecular designs of cytokine armors in the multiple myeloma animal model described in FIG. 13B. In vivo IFN-γ (FIG. 14I), TNF-α (FIG. 14J) and GM-CSF (FIG. 14K) cytokine release from γδ T cells with different molecular designs of cytokine armors in the multiple myeloma animal model described in FIG. 13C. In vivo IFN-γ (FIG. 14L) and GM-CSF (FIG. 14M) cytokine release from γδ T cells with different molecular designs of cytokine armors in the multiple myeloma animal model described in FIG. 13D.

The following detailed description, given by way of example, but not intended to limit the invention solely to the specific embodiments described, may best be understood in conjunction with the accompanying drawings.

### DETAILED DESCRIPTION OF THE INVENTION

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dibel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

All embodiments provided throughout this application are non-limiting embodiments which are given for illustration purposes only and are not intended to limit the invention in any way.

Different technical features, technical solutions, and/or embodiments that are discussed in the same or different aspects/parts of the present application can be combined to form new features, solutions, or embodiments. These new features, solutions, or embodiments also fall into the scope of the present invention.

The present disclosure, in an aspect, provides an engineered γδ T cell comprising:
(i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

The present disclosure, in an aspect, provides an engineered γδ T cell comprising:
(i) a chimeric antigen receptor (CAR) comprising an extracellular antigen recognition domain that is selective for a target, a transmembrane domain, and an intracellular signaling domain, and/or
a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) an exogenous cytokine IL-18 or a functional variant thereof, or a chimeric cytokine receptor comprising the endodomain of the IL-18 receptor (IL-18R).

In some embodiments, the engineered γδ T cell comprises: (i) an anti-BCMA CAR, or an anti-BCMA TCR, or an anti-BCMA antigen recognition domain fused to the CD3 chain of a TCR complex; and (ii) an exogenous cytokine IL-18 or a functional variant thereof, or a chimeric cytokine receptor comprising the endodomain of the IL-18 receptor (IL-18R). In some embodiments, the anti-BCMA CAR is a tandem CAR, for example, comprising more than one, e.g. 2, 3, 4, 5, or 6, antigen recognition portions, e.g. single domain antibodies (sdAb). In some embodiments, the anti-BCMA CAR is a dual CAR, e.g. targeting BCMA, CD19 and GPC3. In some embodiments, IL-18 is in soluble form or a membrane-bound form.

In some embodiments, the engineered γδ T cell comprises: (i) an anti-CD19 CAR, or an anti-CD19 TCR, or an anti-CD19 antigen recognition domain fused to the CD3 chain of a TCR complex; and (ii) an exogenous cytokine IL-18 or a functional variant thereof, or a chimeric cytokine receptor comprising the endodomain of the IL-18 receptor (IL-18R). In some embodiments, the anti-CD19 CAR is a tandem CAR, for example, comprising more than one, e.g. 2, 3, 4, 5, or 6, antigen recognition portions, e.g. single domain antibodies (sdAb). In some embodiments, the anti-CD19 CAR is a dual CAR, e.g. targeting BCMA, CD19 and GPC3. In some embodiments, IL-18 is in soluble form or a membrane-bound form.

In some embodiments, the engineered γδ T cell comprises: (i) an anti-GPC3 CAR, or an anti-GPC3 TCR, or an anti-GPC3 antigen recognition domain fused to the CD3 chain of a TCR complex; and (ii) an exogenous cytokine IL-18 or a functional variant thereof, or a chimeric cytokine receptor comprising the endodomain of the IL-18 receptor (IL-18R). In some embodiments, the anti-GPC3 CAR is a tandem CAR, for example, comprising more than one, e.g. 2, 3, 4, 5, or 6, antigen recognition portions, e.g. single domain antibodies (sdAb). In some embodiments, the anti-GPC3 CAR is a dual CAR, e.g. targeting BCMA, CD19 and GPC3. In some embodiments, IL-18 is in soluble form or a membrane-bound form.

In some embodiments, the engineered γδ T cell comprises a nucleic acid having a nucleotide sequence at least about 80%, 85%, 90%, 91%, 92%, 93%, 94%, 95%, 96%, 97%, 98%, or 99% identical to SEQ ID NO: 14, 17, 19, 20 or 22. In some embodiments, the engineered γδ T cell comprises a nucleic acid having the nucleotide sequence set forth in SEQ ID NO: 14, 17, 19, 20 or 22. In some embodiments, the engineered γδ T cell comprises a nucleic acid of SEQ ID NO: 14. In some embodiments, the engineered γδ T cell comprises a nucleic acid of SEQ ID NO: 17. In some embodiments, the engineered γδ T cell comprises a nucleic acid of SEQ ID NO: 19. In some embodiments, the engineered γδ T cell comprises a nucleic acid of SEQ ID NO: 20. In some embodiments, the engineered γδ T cell comprises a nucleic acid of SEQ ID NO: 22.

In some embodiments, the engineered γδ T cell comprises a polypeptide having an amino acid sequence at least about 80%, 85%, 90%, 91%, 92%, 93%, 94%, 95%, 96%, 97%, 98%, or 99% identical to SEQ ID NO: 2, 5, 7, 8 or 10. In some embodiments, the engineered γδ T cell comprises a polypeptide having the amino acid sequence set forth in SEQ ID NO: 2, 5, 7, 8 or 10. In some embodiments, the engineered γδ T cell comprises an amino acid sequence of SEQ ID NO: 2. In some embodiments, the engineered γδ T cell comprises an amino acid sequence of SEQ ID NO: 5. In some embodiments, the engineered γδ T cell comprises an amino acid sequence of SEQ ID NO: 7. In some embodiments, the engineered γδ T cell comprises an amino acid sequence of SEQ ID NO: 8. In some embodiments, the engineered γδ T cell comprises an amino acid sequence of SEQ ID NO: 10.

Chimeric Antigen Receptors (CARs)

The present invention can be used with any CAR, including but not limited to what are referred to as first-generation, second-generation, third-generation, and “armored” CARs.

The term “chimeric antigen receptor” or “CAR” as used herein refers to an artificially constructed hybrid protein or polypeptide containing a binding moiety (e.g. an antibody) linked to immune cell (e.g. T cell) signaling or activation domains. In some embodiments, CARs are synthetic receptors that retarget T cells to tumor surface antigens (Sadelain et al., Nat. Rev. Cancer 3(1):35-45 (2003); Sadelain et al., Cancer Discovery 3(4):388-398 (2013)). CARs can provide both antigen binding and immune cell activation functions onto an immune cell such as a T cell. CARs have the ability to redirect T-cell specificity and reactivity toward a selected target in a non-MHC-restricted manner, exploiting the antigen-binding properties of monoclonal antibodies. The non-MHC-restricted antigen recognition can give T-cells expressing CARs the ability to recognize an antigen independent of antigen processing, thus bypassing a mechanism of tumor escape.

In certain embodiments, the chimeric receptor comprises an extracellular antigen recognition domain specific for one or more antigens (such as tumor antigens) or epitopes, a transmembrane domain, and an intracellular signaling domain of a T cell, γδ T cell, NK cell or NKT cell and/or co-stimulatory receptors. When used in reference with “antigen recognition domain”, the phrase “selective for a target” or the like means the antigen recognition domain is specific for a target such as a tumor antigen, or has some specificity or selectivity to a target.

“CAR γδ T cell” refers to a γδ T cell that expresses a CAR. “Anti-CD19 CAR” refers to a CAR having an extracellular binding domain specific for CD19, “anti-BCMA CAR” refers to a CAR having an extracellular binding domain specific for BCMA, “anti-GPC3 CAR” refers to a CAR having an extracellular binding domain specific for GPC3, and so on.

Several “generations” of CARs have been developed. First-generation CAR T-cells utilize an intracellular domain from the CD3ζ-chain of the TCR, which provides so called ‘signal 1,’ and induces cytotoxicity against targeted cells. Engagement and signaling via the CD3ζ chain is required for T-cell stimulation and proliferation but is not often sufficient for sustained proliferation and activity in the absence of a second signal or ‘signal 2.’ Second-generation CARs were developed to enhance efficacy and persistence in vivo after reinfusion into a subject and contain an second costimulatory signaling domain (CD28 or 4-1BB) intracellular domain that functions to provide ‘signal 2’ to mitigate anergy and activation-induced cell death seen with first generation CAR T-cells. Third-generation CARs are further optimized by use of two distinct costimulatory domains in tandem, e.g., CD28/4-1BB/CD3 or CD28/OX-40/CD3. (see, e.g., Yeku et al., 2016, Armored CAR T-cells: utilizing cytokines and pro-inflammatory ligands to enhance CAR T-cell anti-tumour efficacy. Biochem Soc Trans. 44(2):412). CARs have been further optimized or “armored” to secrete active cytokines or express costimulatory ligands that further improve efficacy and persistence.

Tandem CAR and Dual CAR

All forms of CARs can be suitably used in the present invention, including but not limited to single CAR, tandem CAR, dual CAR, and the combinations thereof.

Tandem CAR includes more than one antigen-binding portions (such as 2, 4, or 6 sdAb or scFv). Typically, tandem CARs may contain monospecific, bivalent antigen-binding moiety, e.g., two identical VHH domains binding BCMA, CD19 or GPC3, or multi-specific, e.g., bispecific bivalent, antigen-binding moiety, e.g., two different VHH domains binding BCMA, CD19 or GPC3, or one VHH domain binding BCMA, CD19 or GPC3 and the other VHH domain binding a molecule other than BCMA, CD19 or GPC3, a transmembrane domain, and an intracellular domain. In another aspect, the CAR of the present disclosure may include a tandem CAR having an extracellular antigen recognition domain including a first binding domain and a second binding domain, wherein the first binding domain fuses to the second binding domain optionally via a linker.

In some embodiments, the CAR used in the present invention is a tandem CAR which comprises: more than one antigen-binding portions (e.g. single domain antibody (sdAb) and/or single chain variable fragment (scFv)) that target different epitopes on BCMA, CD19 or GPC3, a transmembrane domain, and an intracellular signaling domain.

Dual CAR can be a combination of any two CARs, in which each of a first CAR and a second CAR may be a single CAR or a tandem CAR, i.e., single CAR/single CAR, single CAR/tandem CAR, or tandem CAR/tandem CAR. The levels of dual CAR T cell signaling may be regulated by manipulating the intracellular domains of each first and second CARs. For example, the intracellular domains of each of the first CAR and the second CAR may contain a co-stimulatory domain, such as CD28, 4-1BB (CD137), ICOS, OX40 (CD134), CD27, and/or DAP10, and/or a signaling domain from a T cell receptor, such as a signaling domain from a T cell receptor (e.g., CD3( ). For example, dual CAR of the present disclosure may include a first CAR and a second CAR each having an intracellular domain containing a co-stimulatory domain and a signaling domain from a T cell receptor. Thus, when dual CAR bind antigens (e.g., bispecific), the T cell signals may be transmitted through two signaling domains from a T cell receptor. Dual CAR of the present disclosure may also include a first CAR having an intracellular domain containing a co-stimulatory domain and a signaling domain from a T cell receptor and a second CAR having an intracellular domain containing a co-stimulatory domain. Thus, when dual CAR bind antigens (e.g., bispecific), the T cell signals may be transmitted through the signaling domain from a T cell receptor of the first CAR.

In some embodiments of the present invention, the tandem CAR or dual CAR targets the same tumor antigen, for example, they can target different epitopes on the same tumor antigen, such as different epitopes of BCMA, CD19 or GPC3. In some embodiments of the present invention, the tandem CAR or dual CAR targets the same tumor antigen, for example, they can target different epitopes on the same tumor antigen, such as different epitopes of BCMA. In some embodiments of the present invention, the tandem CAR or dual CAR targets the same tumor antigen, for example, they can target different epitopes on the same tumor antigen, such as different epitopes of CD19. In some embodiments of the present invention, the tandem CAR or dual CAR targets the same tumor antigen, for example, they can target different epitopes on the same tumor antigen, such as different epitopes of GPC3. In some embodiments, the tandem CAR or dual CAR targets different tumor antigens, such as BCMA, CD19 and GPC3.

CAR Ligand-Binding Domains

CARs typically employ scFv domains of antibodies to target cell surface antigens of target cells. These binding domains consist of a variable heavy and variable light chains fused together with a flexible linker. The variable domains are derived within an antibody, determining antigen specificity. TCR-like antibody based CARs are a class of CARs which express scFvs from antibodies that specifically recognize MHC class molecules and its loaded peptide (Dahan et al., 2012, T-cell-receptor-like antibodies-generation, function and applications. Expert Reviews in Molecular Medicine. 14:e6). This specificity can be utilized to target cancers based on recognition of mutated intracellular proteins. If mutated peptide sequences are loaded onto the MHC, they could effectively generate neo-epitopes, which can be used to distinguish a cancerous cell from a normal cell by a CAR that only recognizes the specific MHC/peptide combination.

The phrases “ligand-binding domain”, “antigen binding domain”, “antigen recognition domain”, and “targeting domain” are used interchangeably with reference to CARs or TCRs in the present application. Antigen recognition domains take many forms. Non-limiting examples include bispecific receptors (Zakaria Grada, et al. TanCAR: A Novel Bispecific Chimeric Antigen Receptor for Cancer Immunotherapy. Molecular Therapy, 2013, 2, e105), single domain VHH based CARs (De Meyer T, et a., VHH-based products as research and diagnostic tools. Trends Biotechnol. 2014 May; 32(5):263-70), and “universal” CARs comprising avidin that binds to any antigen receptor that incorporates biotin (Huan Shi, et al. Chimeric antigen receptor for adoptive immunotherapy of cancer: latest research and future prospects. Molecular Cancer, 2014,13:219).

The term “antigen recognition domain” as used herein refers to an antibody fragment including, but not limited to, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single domain antibody (sdAb), a single chain variable fragment (scFv) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen recognition domain is capable of binding to the same antigen to which the parent antibody or a parent antibody fragment (e.g., a parent scFv) binds. In some embodiments, an antigen-binding fragment may comprise one or more complementarity determining regions (CDRs) from a particular human antibody grafted to frameworks (FRs) from one or more different human antibodies.

The antigen recognition domain can be made specific for any disease-associated antigen, including but not limited to tumor antigens (for example, tumor-associated antigens (TAAs) or tumor-specific antigen (TSA)) and infectious disease-associated antigens. In certain embodiments, the extracellular antigen recognition domain is selective for a tumor antigen or an infectious disease-associated antigen.

In certain embodiments, the antigen recognition domain is multispecific, such as bispecific or trispecific. The term “multispecific” is used in the present disclosure in its broader sense, which is, an antigen recognition domain is multispecific if it can target more than one epitopes on the same antigen or it can target more than one antigens.

Antigens have been identified in most of the human cancers, including Burkitt lymphoma, neuroblastoma, melanoma, osteosarcoma, renal cell carcinoma, breast cancer, prostate cancer, lung carcinoma, and colon cancer. TAAs include, without limitation, CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP and HER-2. TAAs further include neoantigens, peptide/MHC complexes, and HSP/peptide complexes. BCMA, i.e. B-cell maturation antigen, is a cell surface protein universally expressed on malignant plasma cells and it has emerged as a very selective antigen to be targeted in novel treatments.

In certain embodiments, the antigen recognition domain comprises a T-cell receptor or binding fragment thereof that binds to a defined tumour specific peptide-MHC complex.

The term “T-cell receptor” or “TCR” refers to a molecule on the surface of a T cell or T lymphocyte that is responsible for recognizing an antigen. TCR is a heterodimer which is composed of two different protein chains. In some embodiments, the TCR of the present disclosure consists of an alpha (α) chain and a beta (β) chain and is referred as αβ TCR. αβ TCR recognizes antigenic peptides degraded from protein bound to major histocompatibility complex molecules (MHC) at the cell surface. In some embodiments, the TCR of the present disclosure consists of a gamma (γ) and a delta (δ) chain and is referred as γδ TCR. γδ TCR recognizes peptide and non-peptide antigens in a MHC-independent manner. γδ T cells have shown to play a prominent role in recognizing lipid antigens. In particular, the γ chain of TCR includes but is not limited to Vγ2, Vγ3, Vγ4, Vγ5, Vγ8, Vγ9, Vγ10, a functional variant thereof, and a combination thereof; and the δ chain of TCR includes but is not limited to δ1, δ2, δ3, a functional variant thereof, and a combination thereof. In some embodiments, the γδ TCR may be Vγ2/Vδ1TCR, Vγ2/Vδ2 TCR, Vγ2/Vδ3 TCR, Vγ3/Vδ1 TCR, Vγ3/Vδ2 TCR, Vγ3/Vδ3 TCR, Vγ4/V1 TCR, Vγ4/VS2 TCR, Vγ4/Vδ3 TCR, Vγ5/Vδ1 TCR, Vγ5/Vδ2 TCR, Vγ5/V83 TCR, Vγ8N81 TCR, Vγ8/Vδ2 TCR, Vγ8/V3 TCR, Vγ9/Vδ1 TCR, Vγ9/Vδ2 TCR, Vγ9/Vδ3 TCR, Vγ10/Vδ1 TCR, Vγ10/Vδ2 TCR, and/or Vγ10/Vδ3 TCR. In some examples, the γδ TCR may be Vγ9/Vδ2 TCR, Vγ10/Vδ2 TCR, and/or Vγ2/Vδ2 TCR.

In certain embodiments, the antigen recognition domain comprises a natural ligand of a tumor expressed protein or tumor-binding fragment thereof. For example, the transferrin receptor 1 (TfR1), also known as CD71, is a homodimeric protein that is a key regulator of cellular iron homeostasis and proliferation. Although TfR1 is expressed at a low level in a broad variety of cells, it is expressed at higher levels in rapidly proliferating cells, including malignant cells in which overexpression has been associated with poor prognosis. In an embodiment of the invention, the antigen recognition domain comprises transferrin or a transferrin receptor-binding fragment thereof.

In certain embodiments, the antigen recognition domain is specific to a defined tumor associated antigen, such as but not limited to BCMA, CD19, GPC3, FRα, CEA, 5T4, CA125, SM5-1 or CD71. In certain embodiments, the tumor associated antigen can be a tumor-specific peptide-MHC complex. In certain such embodiments, the peptide is a neoantigen. In other embodiments, the tumor associated antigen it a peptide-heat shock protein complex.

In certain embodiments, targeting domains of CARs of the invention target tumor-associated antigens. In certain embodiments, the tumor-associated antigen is selected from: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (ela2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAGIB, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OAl, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

Intracellular Signaling Domain

The intracellular signaling domain comprises a primary intracellular signaling domain of an immune effector cell (such as T cell, e.g. γδ T cell). In certain embodiments, the primary intracellular signaling domain is derived from CD3ζ, FcRγ, FcRβ, CD3γ, CD3δ, CD3s, CD5, CD22, CD79a, CD79b, or CD66d. In certain embodiments, the primary intracellular signaling domain is derived from CD3 (i.e., “a CD3 intracellular signaling domain”). In certain embodiments, the intracellular signaling domain comprises an intracellular co-stimulatory sequence. In certain embodiments, the intracellular signaling domain comprises both a primary intracellular signaling domain (e.g., a CD3(intracellular signaling domain) and an intracellular co-stimulatory domain. In certain embodiments, the intracellular signaling domain comprises a primary intracellular signaling domain but does not comprise an intracellular co-stimulatory domain. In certain embodiments, the intracellular signaling domain comprises an intracellular co-stimulatory sequence but does not comprise a primary intracellular signaling domain.

Co-stimulatory Domains

“Co-stimulatory domain” (CSD) as used herein refers to the portion of the CAR which enhances the proliferation, survival and/or development of memory cells. The CARs of the invention may comprise one or more co-stimulatory domains. Each costimulatory domain comprises a costimulatory domain of any one or more of, for example, members of the TNFR superfamily, CD28, CD137 (4-1BB), CD134 (OX40), Dap10, CD27, CD2, CD5, ICAM-1, LFA-l(CD1 1a/CD18), Lck, TNFR-I, TNFR-II, Fas, CD30, CD40 or combinations thereof. Further costimulatory domains used with the invention comprise one or more of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLγδ/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8α, CD8β, CD82/Kai-1, CD84/SLAMF5, CD90/Thy1, CD96, CDS, CEACAMI, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R P, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSFI9L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6.

In certain embodiments, the intracellular signaling domain comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.

Transmembrane Domain

“Transmembrane domain” (TMD) as used herein refers to the region of the CAR which crosses the plasma membrane. The transmembrane domain of the CAR of the invention is the transmembrane region of a transmembrane protein (for example Type I transmembrane proteins), an artificial hydrophobic sequence or a combination thereof. Although the main function of the transmembrane is to anchor the CAR in the T cell membrane, in certain embodiments, the transmembrane domain influences CAR function. In certain embodiments, the transmembrane domain is from CD4, CD8α, CD28, or ICOS. Gueden et al. associated use of the ICOS transmembrane domain with increased CAR T cell persistence and overall anti-tumor efficacy (Guedan S. et al., Enhancing CAR T cell persistence through ICOS and 4-1BB costimulation. JCI Insight. 2018; 3:96976). In an embodiment, the transmembrane domain comprises a hydrophobic a helix that spans the cell membrane. Other transmembrane domains will be apparent to those of skill in the art and may be used in connection with alternate embodiments of the invention. In certain embodiments, the transmembrane domain is a human transmembrane domain. In certain embodiments, the transmembrane domain comprises human CD8a transmembrane domain. In certain embodiments, the transmembrane domain comprises human CD28 transmembrane domain.

Hinge Region

The chimeric receptors of the present application may comprise a hinge domain that is located between the extracellular antigen recognition domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular domain relative to the transmembrane domain of the effector molecule can be used. The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In certain embodiments, the hinge domain is a hinge domain of a naturally occurring protein. Hinge domains of any protein known in the art to comprise a hinge domain are compatible for use in the chimeric receptors described herein. In certain embodiments, the hinge domain is at least a portion of a hinge domain of a naturally occurring protein and confers flexibility to the chimeric receptor. In certain embodiments, the hinge domain is derived from CD8, such as CD8α. In certain embodiments, the hinge domain is a portion of the hinge domain of CD8α, e.g., a fragment containing at least 15 (e.g., 20, 25, 30, 35, or 40) consecutive amino acids of the hinge domain of CD8α. In certain embodiments, the hinge domain is derived from CD28.

Hinge domains of antibodies, such as an IgG, IgA, IgM, IgE, or IgD antibodies, are also compatible for use in the chimeric receptor systems described herein. In certain embodiments, the hinge domain is the hinge domain that joins the constant domains CH1 and CH2 of an antibody. In certain embodiments, the hinge domain is of an antibody and comprises the hinge domain of the antibody and one or more constant regions of the antibody. In certain embodiments, the hinge domain comprises the hinge domain of an antibody and the CH3 constant region of the antibody. In certain embodiments, the hinge domain comprises the hinge domain of an antibody and the CH2 and CH3 constant regions of the antibody. In certain embodiments, the antibody is an IgG, IgA, IgM, IgE, or IgD antibody. In certain embodiments, the antibody is an IgG antibody. In some embodiments, the antibody is an IgG1, IgG2, IgG3, or IgG4 antibody. In certain embodiments, the hinge region comprises the hinge region and the CH2 and CH3 constant regions of an IgG1 antibody. In certain embodiments, the hinge region comprises the hinge region and the CH3 constant region of an IgG1 antibody.

Non-naturally occurring peptides may also be used as hinge domains for the chimeric receptors described herein. In certain embodiments, the hinge domain between the C-terminus of the extracellular ligand-binding domain of an Fc receptor and the N- terminus of the transmembrane domain is a peptide linker, such as a (GxS)n linker, wherein x and n, independently can be an integer between 3 and 12, including 3, 4, 5, 6, 7, 8, 9, 10, 11, 12, or more.

In certain embodiments, both the first nucleic acid and the second nucleic acid have a leading peptide.

Promoters

In some embodiments, the first polynucleotide is operably linked to a first promoter, and the second polynucleotide is operably linked to a second promoter. In some embodiments, the first polynucleotide and the second polynucleotide are linked to the same promoter. In some embodiments, the first polynucleotide and the second polynucleotide are operably linked to each other via a third polynucleotide encoding a self-cleaving peptide, such as T2A, P2A, or F2A. In some embodiments, the self-cleaving peptide is P2A.

A large number of promoters recognized by a variety of potential host cells are well known. Any promoter suitable for the practice of the present invention can be used herein.

One example of a suitable promoter for CAR, TCR or antigen recognition domain fused to CD3 chain of TCR complex is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumour virus (MMTV), human immunodeficiency virus (HIV) long terminal repeat (LTR) promoter, MoMuLV promoter, an avian leukaemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Exemplary promoters for cytokine expression include but are not limited to an IFN-3 promoter, an IL-2 promoter, a BCL-2 promoter, a GM-CSF promoter, an IL-6 promoter, an IFN-γ promoter, an IL-12 promoter, an IL-4 promoter, an IL-15 promoter, an IL-18 promoter or an IL-21 promoter.

Promoters typically fall into two classes, inducible and constitutive, both of which are contemplated in the present invention. Inducible promoter is a promoter that initiates increased levels of transcription under its control in response to changes in the condition, e.g. the presence or absence of a nutrient or other chemicals.

In certain embodiments, cytokine expression is driven by an IFN-β promoter or functional promoter fragment thereof. The IFN-β promoter is well known and characterized (see, e.g, Vodjdani G. et al., 1988. Structure and characterization of a murine chromosomal fragment containing the interferon beta gene. J Mol Biol. 204(2):221-31) and an IFN-β promoter fragment sufficient to drive cytokine expression is exemplified herein.

In certain embodiments, cytokine expression is driven by an IL-2 promoter or functional promoter fragment thereof. The T cell growth factor, IL-2, is the major cytokine that is produced during the primary response of T cells. IL-2 expression is controlled tightly at the transcriptional level, and extensive analysis of the IL-2 gene established a minimal promoter region, which extends about 300 bp relative to the transcription start site, that is known to be sufficient for IL-2 induction upon T cell activation in vitro. (Jain, J. et al., 1995, Transcriptional regulation of the IL-2 gene. Curr. Opin. Immunol. 7:333-342; Serfling, E. et al., 1995, The architecture of the interleukin-2 promoter: a reflection of T lymphocyte activation. Biochim. Biophys. Acta. 1263:181-200).

In certain embodiments, cytokine expression is driven by a BCL-2 promoter or functional promoter fragment thereof. In certain embodiments, the promoter fragment is a minimal BCL-2 promoter.

In certain embodiments, cytokine expression is driven by a GM-CSF promoter or functional promoter fragment thereof. In certain embodiments, the promoter fragment is a minimal GM-CSF promoter.

In certain embodiments, cytokine expression is driven by an IL-6 promoter or functional promoter fragment thereof. In certain embodiments, the promoter fragment is a minimal IL-6 promoter.

In certain embodiments, cytokine expression is driven by an IFN-γ promoter or functional promoter fragment thereof. In certain embodiments, the promoter fragment is a minimal IFN-γ promoter.

In certain embodiments, cytokine expression is driven by an IL-12 promoter or functional promoter fragment thereof. In certain embodiments, the promoter fragment is a minimal IL-12 promoter.

In certain embodiments, cytokine expression is driven by an IL-4 promoter or functional promoter fragment thereof. In certain embodiments, the promoter fragment is a minimal IL-4 promoter.

In certain embodiments, cytokine expression is driven by an IL-18 promoter or functional promoter fragment thereof. In certain embodiments, the promoter fragment is a minimal IL-18 promoter.

In certain embodiments, cytokine expression is driven by an IL-21 promoter or functional promoter fragment thereof. In certain embodiments, the promoter fragment is a minimal IL-21 promoter.

Minimal Promoters

Minimal promoters are described in the art and may be selected to minimize the basal level of transcription in cell that are not activated. For example, Parvin et al. describes a eukaryotic minimal promoter of IgH transcription that can be reconstitute in vitro in a minimal reaction that contains only TATA-binding protein (TPB), TFIIB and RNA polymerase II (pol II) when the template is negatively coiled. (Parvin et al., 1993, DNA topology and a minimal set of basalfactors for transcription by RNA polymerase II. Cell 73:522). Butler (Butler et al, 2002, The RNA polymerase II core promoter: a key component in the regulation of gene expression. Genes & Dev. 16:2583) refers to the core promoter as the minimal stretch of contiguous DNA sequence that is sufficient to direct accurate initiation of transcription by the RNA polymerase II machinery. According to Butler, a core promoter typically encompasses the site of transcription initiation and extends either upstream or downstream for an additional ˜35 nucleotides and in many instances will comprise only about 40 nt, include the TATA box, initiator (Inr), TFIIB recognition element (BRE), and downstream core promoter element (DPE) that are commonly found in core promoters but also notes that each of these core promoter elements is found in some but not all core promoters. These are distinct from other cis-acting DNA sequences that regulate RNA polymerase II transcription such as the proximal promoter, enhancers, silencers, and boundary/insulator elements which contain recognition sites for a variety of sequence-specific DNA-binding factors that are involved in transcriptional regulation. The proximal promoter is the region in the immediate vicinity of the transcription start site (roughly from −250 to +250 nt). Enhancers and silencers can be located many kbp from the transcription start site and act either to activate or to repress transcription.

Transcription Factor Binding Sites

In some embodiments, the expression of the nucleic acid encoding the armor (i.e. an exogenous IL-18 or a IL-18 chimeric cytokine receptor) in the CAR (or TCR) γδ T cell where it was introduced into is regulated using promoters and transcription factor binding sites that are active and can be modulated once the immune cell is activated, e.g. upon engagement of the CAR or TCR with an antigen.

NFκb and AP-1 are transcriptional factors that play an important role in gene transcription in activated immune cells. Both TCR and CAR based signaling pathways activate NFκb and AP-1 transcriptional factors. T cell-NF-κB plays an important role in tumor control. It is also investigated that stimulation of NK cells or γδ T cells with specific cell targets results in an increased binding activity of NF-κB and AP-1 transcription factors.

When the immune cell is activated by antigen engagement there is activation and nuclear translocation of activator protein-1 (AP-1) and nuclear factor-x-light chain enhancer of activated B cells (NF-κB) transcriptional factors, which bind to their respective sites at the promoter to stimulate transcription. Thus, a cytokine encoding sequence or other sequence operatively linked to a promoter and transcription factor binding sites for AP-1, NF-κB, or other transcription factor that operates at the binding site when the cell is activate is expressed at high levels when the cell is activated and at low or undetectable levels when the cell is not activated.

The NF-κB transcription factor family in mammals consists of five proteins, p65 (RelA), RelB, c-Rel, p105/p50 (NF-κB1), and p100/52 (NF-κB2) that associate with each other to form distinct transcriptionally active homo- and heterodimeric complexes. They all share a conserved 300 amino acid long amino-terminal Rel homology domain (RHD), and sequences within the RHD are required for dimerization, DNA binding, interaction with 1κBs, as well as nuclear translocation. (Oeckinghaus et al., 2009, The NF-κB Family of Transcription Factors and Its Regulation, Cold Spring Harb Perspect Biol. 2009 October; 1(4): a000034).

NF-κB exerts its fundamental role as transcription factor by binding to variations of the consensus DNA sequence of 5′-GGGRNYYYCC-3′ (in which R is a purine, (i.e., A or G), Y is a pyrimidine (i.e., C or T), and N is any nucleotide) known as KB sites. How NF-κB selectively recognizes a small subset of relevant KB sites from the large excess of potential binding sites (about 1.4×10⁴ estimated in human genome) is a critical step for stimulus-specific gene transcription. At a molecular level, DNA-binding differences of individual NF-κB dimers have been linked to dimer-specific roles in gene regulation (Hoffmann et al., 2006, Transcriptional regulation via the NF-kappaB signaling module. Oncogene 25:6706; Natoli G., 2006, Tuning up inflammation: how DNA sequence and chromatin organization control the induction of inflammatory genes by NF-kappaB. FEBS Lett. 580:2843). Much work has been carried out to identify structural features of NF-κB: DNA complexes and how distinctive features of NF-κB proteins and DNA sequences contribute to specific complex formation (Siggers et al., 2012, Principles of dimer-specific gene regulation revealed by a comprehensive characterization of NF-κB family DNA binding. Nat Immunol. 13(1): 95; Mulero et al., 2019, Genome reading by the NF-κB transcription factors. Nucleic Acids Res. 47(19):9967). The presence of NF-κB sites is observed to be a minimal requirement for NF-κB regulation but not sufficient for gene induction (Wan et al., 2009, Specification of DNA Binding Activity of NF-κB Proteins, Cold Spring Harb Perspect Biol. 1(4): a000067.).

The dimeric transcription factor complex Activator Protein-1 (AP-1) is a group of proteins involved in a wide array of cell processes and a critical regulator of nuclear gene expression during T-cell activation. AP-1 transcription factors are homo- or hetero-dimmer forming proteins that belong to a group of DNA binding proteins called Basic-Leucine Zipper domain (bZIP) proteins. Dimerization between members of the AP-1 family occurs through a structure which is known as leucine zipper, comprised of a heptad of repeats of leucine residues along a α-helix, which can dimerize with another α-helix via formation of a coiled-coil structure with contacts between hydrophobic leucine zipper domain. Adjacent to the leucine zipper lies a basic DNA binding domain which is rich in basic amino acids and is responsible for DNA-binding in either 12-O-tetradecanoylphorbol-13-acetate (TPA) response elements (5′-TGAG/CTCA-3′) or cAMP response elements (CRE, 5′-TGACGTCA-3′) (Shaulian et al. AP-1 as a regulator of cell life and death. Nat. Cell Biol. 4:E131; Atsaves, 2019, AP-1 Transcription Factors as Regulators of Immune Responses in Cancer. Cancers 11(7):1037).

The Myc proteins (c-Myc, L-Myc, S-Myc, and N-Myc) are a family of transcription factors that regulate growth and cell cycle entry by their ability to induce expression of genes required for these processes. In normal cells, mitogen stimulation leads to a burst of Myc expression in G1 phase, facilitating entry into the cell cycle. MYC plays a role in regulating a range of innate and adaptive immune cells, and is a key transcription factor that regulates immune cell maturation, development, proliferation and activation, including macrophages, T cells, dendritic cells, and natural killer (NK) cells.

Another useful transcriptional control mechanism of the invention involves the NR4A1 family of transcription factors (e.g., NR4A1, NR4A2 and NR4A3). When NR4A1 is overexpressed in naive T cells, there is upregulation of genes related to anergy and exhaustion, downregulation of genes related to effector programs, reduced TH1 and TH17 differentiation in CD4+ T cells, and reduced IFNγ production by CD8⁺ T cells. Ablation of NR4A1 enhances effector functions of CD4⁺ and CD8⁺ T cells, increases expansion, and blocks the formation of tolerance. (Liu X. et al., 2019, Genome-wide analysis identifies NR4A1 as a key mediator of T cell dysfunction. Nature. 2019 Feb 27). According to the invention, NR4A is a useful transcription factor to maintain expression of cytokines. Incorporation of NR4A binding elements in constructs of the invention boosts cytokine expression and prolongs cytokine release by the CAR T cells.

Similarly, TOX transcription factors act as mediators of T cell exhaustion. TOX and TOX2 as well as NR4A family members have been shown to be highly induced in CD8⁺ CAR⁺ PD-1^{high} TIM3^{high}(“exhausted”) TILs. (Seo, H. et al., 2019, TOX and TOX2 transcription factors cooperate with NR4A transcription factors to impose CD8⁺ T cell exhaustion, PNAS Jun. 18, 2019 116 (25):12410). Other TOX family members include TOX3 and TOX4. TOX transcription factors normally activate transcription through cAMP response element (CRE) sites and protect against cell death by inducing antiapoptotic and repressing pro-apoptotic transcripts. According to the invention, TOX family binding elements are used to increase and/or prolong cytokine expression. An example of a cAMP response element (CRE) is the response element for CREB which contains the highly conserved nucleotide sequence, 5′-TGACGTCA-3′.

Another group of useful transcription factors involved in transcription activation in immune cells are members of signal transducer and activator of transcription (STAT) family proteins, including STAT3, STAT4, STAT5A, STAT5B, and, STAT6, which mediate response to cytokines and growth factors. STAT proteins dimerize through reciprocal pTyr-SH2 domain interactions, and translocate to the nucleus where they bind to specific STAT-response elements in the target gene promoters and regulate transcription. There are 10 or so STAT-response elements, in general consisting of a palindromic sequence, TT Nᵢ AA, where i is 4, 5, or 6. Recognition of this sequence by a particular STAT depends on the value of i as well as on the specific sequence for Nᵢ. For example, binding of STAT3 is better if N is 4, STAT1 if N is 5, and STAT6 if N is 6. (Schindler, U. et al., 1995, Components of a Stat recognition code: evidence for two layers of molecular selectivity. Immunity 2: 689.; Seidel, H. M. et al., 1995, Spacing of palindromic half sites as a determinant of selective STAT (signal transducers and activators of transcription) DNA binding and transcriptional activity. Proc. Natl. Acad. Sci. USA 92:3041).

The transcription factor binding sites can be used singly or in multiples, for example 1, 2, 3, 4, 5, 6, 7, 8, 9, 10, or more transcription factor binding sites. The transcription factors can be the same or different, and can be mixed in varying ratios and in any order. Exemplary constructs comprise 5 sequential NF-κB binding sites with 1 AP binding site, and 3 sequential NF-κB binding sites with 1 AP binding site.

Leading Peptide

The chimeric receptors of the present application may comprise a leading peptide (also known as a signal sequence) at the N-terminus of the polypeptide. In general, leading peptides are peptide sequences that target a polypeptide to the desired site in a cell. Leading peptides including signal sequences of naturally occurring proteins or synthetic, non-naturally occurring signal sequences may be compatible for use in the chimeric receptors described herein. In some embodiments, the leading peptide is derived from a molecule selected from the group consisting of CD8, GM-CSF receptor α, and IgG1 heavy chain. In some embodiments, the signal peptide is derived from CD8, such as CD8α.

T Cell Receptor (TCR)

The T-cell receptor (TCR) is a protein complex found on the surface of T cells that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR.

The structure and function of TCR have been extensively discussed in publications. The TCR is a hetero dimer composed of two different protein chains. In humans, in 95% of T cells the TCR consists of an a chain and a β chain, whereas in 5% of T cells the TCR consists of γ and δ chains. All types of TCR can be utilized in the present invention.

The definition and discussion in connection with the extracellular antigen recognition domain of CARs also apply to the antigen recognition domain that is fused to the CD3 chain of a TCR complex in the present invention. The TCR complex used in the present invention comprises (a) a TCR chain selected from a gamma chain and a delta chain of a T cell receptor, (b) an epsilon chain, a delta chain, and/or a gamma chain of CD3, or (c) a zeta chain of CD3.

IL-18, IL-18R, and chimeric cytokine receptor (CCR)

Unless otherwise indicated, the term “cytokine” used herein refers to interleukin IL-18.

Interleukin-18 (IL-18) is a member of the IL-1 family of cytokines. Many lines of evidence indicate that IL-18 plays an important role in the pathogenesis of inflammatory diseases. IL-18R, the receptor of IL-18, belongs to the IL-1R family, and the IL-18R complex is composed of the IL-18Rα and IL-18β chains. IL-18 exerts its biological function by binding with IL-18R.

The genetically engineered γδ T cells according to the present invention may be further armored by IL-18. The armor can be an interleukin IL-18 or functional variants thereof; or alternatively, it can be a chimeric cytokine receptor comprising the endodomain of IL-18R (IL-18Ra and/or IL-18Rβ) which is also called “the IL-18 based chimeric cytokine receptor” in the present disclosure.

A chimeric cytokine receptor (CCR) is a molecule which comprises a cytokine receptor endodomain and a heterologous ligand-binding exodomain. The heterologous exodomain binds a ligand other than the cytokine for which the cytokine receptor from which the endodomain was derived is selective. In this way, it is possible to alter the ligand specificity of a cytokine receptor by grafting on a heterologous binding specificity.

Generally a chimeric cytokine receptor may comprise: (i) a ligand binding exodomain; (ii) an optional spacer; (iii) a transmembrane domain; and (iv) a cytokine-receptor endodomain.

An “IL-18 based chimeric cytokine receptor” is a chimeric cytokine receptor that comprises the endodomain of IL-18R (IL-18Rα, IL-18Rβ, or the combination thereof). It may comprise an exodomain of a cytokine other than IL-18 (e.g. IL-4, IL-7, IL-15, IL-21, and so on), therefore the function or the functioning level of IL-18R can be regulated through activities on the exodomain (e.g. by engaging the exodomain with an antigen or other moieties such as small molecules). In accordance with the same principle or mechanism, the exodomain of the chimeric cytokine receptor of the present invention can be replaced with an artificial ligand, e.g. PD-L1 ligand (Programmed Death Ligand-1). For example, the artificial ligand can engage with an antigen or other moieties, or it can respond to a chemical (e.g. a medicinal agent), so that the function of the artificial ligand is regulated or can be modified, which in turn regulating or modifying the function of the endodomain of the chimeric cytokine receptor.

To be fully functioning, a chimeric cytokine receptor may also comprise a transmembrane domain, and preferably a dimerization domain to form a heterodimer between the IL-18Rα chain and the IL-18Rβ chain or a homodimer between two IL-18Rα chains or between two IL-18Rβ chains.

The endodomain of the IL-18 based CCR is a signaling domain which comprises the endodomain of IL-18Rα, the endodomain of IL-18R3, or the endodomains of both IL-18Rα and IL-18Rβ. Specifically, the endodomain of the IL-18 based CCR can comprise a Toll/interleukin-1 receptor homology (TIR) domain and an adaptor domain.

Therefore, in some embodiments of the present invention, the IL-18 based chimeric cytokine receptor comprises an ligand binding exodomain, a transmembrane domain, a dimerization domain, and an endodomain, wherein the ligand binding exodomain is from a cytokine other than IL-18 (e.g. IL-4, IL-7, IL-15, IL-21, and so on), or it can be an artificial ligand; the endodomain is from IL-18Rα or IL-18Rβ or both. In some embodiment, the endodomain may comprise a Toll/interleukin-1 receptor homology (TIR) domain and an adaptor domain.

In some embodiments, the genetically engineered γδ T cells according to the present invention comprise an exogenous cytokine IL-18 polypeptide or a nucleic acid encoding an exogenous cytokine IL-18 polypeptide. As used herein, the term “exogenous” is intended to mean that the referenced molecule or other material is introduced into, or non-native to, the host cell, tissue, organism, or system. The molecule can be introduced, for example, by introduction of an encoding nucleic acid into the host genetic material such as by integration into a host chromosome or as non-chromosomal genetic material such as a plasmid.

Nucleic Acids

In an aspect, the present disclosure provides genetically engineered γδ T cell which comprises and expresses the following two nucleic acids: (i) a first nucleic acid encoding a CAR, TCR, and/or an antigen binding domain fused to the CD3 chain of a TCR complex, and (ii) a second nucleic acid encoding an exogenous cytokine IL-18 or IL-18 based chimeric cytokine receptor. Each of the first and second nucleic acids can be constitutively or inducibly expressed. Any form of IL-18 can be used, e.g. full length polypeptide or a fragment thereof, soluble or membrane-bound. This genetic modification/manipulation produces a CAR (or TCR) γδ T cell armored with interleukin IL-18, which has multiple advantages for cancer treatment or related uses, and can also serve as a platform to make further genetic modifications.

In an embodiment, the engineered γδ T cell of the present invention comprises: (i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain; and/or a first nucleic acid, which comprises a first nucleic acid sequence that encodes a T cell receptor (TCR) or antigen recognition domain fused to the CD3 chain of a TCR complex, where the TCR complex comprising (a) a TCR chain selected from an alpha chain, a beta chain, a gamma chain and a delta chain of a T cell receptor, (b) an epsilon chain, a delta chain, and/or a gamma chain of CD3, or (c) a zeta chain of CD3; and (ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

In certain embodiments, the first nucleic acid further comprises a first regulatory region which comprises a promoter operatively linked to the first nucleic acid sequence, for the expression of the first nucleic acid sequence.

In certain embodiments, the second nucleic acid further comprises a second regulatory region operatively linked to the second nucleic acid sequence, for the expression of the second nucleic acid sequence. In certain embodiments, the second regulatory region comprises (i) an inducible promoter, and/or (ii) a promoter and one or more transcription factor binding sites, wherein the transcription factor binding sites bind to transcription factors that are active in activated γδ T cells.

In certain embodiments, the first nucleic acid and the second nucleic acid are linked and comprised in a vector, and they can be transcribed in the same or opposite directions. In other embodiments, the first nucleic acid and the second nucleic acid are comprised in separate vectors, and they can be introduced to the cell separately. Said vector can be any vehicle that can be advantageously utilized to introduce nucleic acids into T cells, including but not limited to a virus vector, e.g. a lentivirus or retrovirus vector.

In some embodiments, the engineered γδ T cell of the present invention comprises:
(i) a first nucleic acid, which comprises a first regulatory region operatively linked to a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from an alpha chain, a beta chain, a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R),
wherein the extracellular antigen recognition domain is selective for a tumor antigen selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof;
the intracellular signaling domain comprises a primary intracellular signaling domain of an immune effector cell derived from a signal transducing molecule selected from the group consisting of CD3ζ, FcRγ, FcRβ, CD3γ, CD36, CD3&, CD5, CD22, CD79a, CD79b, CD66d and combinations thereof; and the intracellular signaling domain further comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof;
the transmembrane domain is from CD4, CD8α, CD28, or ICOS; and
optionally, the second nucleic acid sequence further comprises a second regulatory region which is inducible and operatively linked to the second nucleic acid sequence.

In some embodiments, the engineered γδ T cell of the present invention comprises:
(i) a first nucleic acid, which comprises a first regulatory region operatively linked to a first nucleic acid sequence that encodes a chimeric antigen receptor (CAR) comprising: more than one tandem antigen recognition portions targeting BCMA, CD19 or GPC3; a transmembrane domain selected from CD4, CD8α, CD28, or ICOS; a CD3(intracellular signaling domain; and a CD28 or 4-1BB intracellular co-stimulatory domain;
and
(ii) a second nucleic acid, which comprises a nucleic acid sequence that encodes an exogenous cytokine IL-18 or a fragment thereof, or a chimeric cytokine receptor comprising the endodomain domain of the IL-18 receptor (IL-18R).

In certain embodiments, the engineered γδ T cell comprises a nucleic acid having a nucleotide sequence at least about 80%, 85%, 90%, 91%, 92%, 93%, 94%, 95%, 96%, 97%, 98%, 99% or 100% identical to SEQ ID NO: 14, 17, 19, 20 or 22. In certain embodiments, the engineered γδ T cell comprises a nucleic acid having a nucleotide sequence of any one of SEQ ID NO: 14, 17, 19, 20 or 22.

As used herein, the terms “polynucleotide”, “nucleotide”, and “nucleic acid” are intended to be synonymous with each other. It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed, e.g. codon optimization. Nucleic acids according to the invention may comprise DNA or RNA. They may be single stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid sequences may be joined by a sequence allowing co-expression of the two or more nucleic acid sequences. For example, the construct may rearranged and comprise an internal promoter. There can be expression of multiple cytokines, using for example, an additional promoter, an internal ribosome entry sequence (IRES) sequence or a sequence encoding a cleavage site. The cleavage site may be self-cleaving, such that when the polypeptide is produced, it is immediately cleaved into the discrete proteins without the need for any external cleavage activity. Various self-cleaving sites are known, including the Foot-and Mouth disease virus (FMDV) and the 2A self-cleaving peptide. The co-expressing sequence may be an internal ribosome entry sequence (IRES). The co-expressing sequence may be an internal promoter.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame).

Variants

As used herein, the phrase “a nucleic acid having a nucleotide sequence at least, for example, 95% ‘identical’ to a reference nucleotide sequence” is intended to mean that the nucleotide sequence of the nucleic acid is identical to the reference sequence except that it can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among Attorney Docket No. 51624-0048US1/Client ref. L2-W20205WU-US nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, amino acid sequence variants are contemplated. The terms “variant”, “homologue” or “derivative” in relation to a polypeptide sequence include any substitution of, variation of, modification of, replacement of, deletion of or addition of one (or more) amino acid from or to the sequence, and “a functional variant” means a variant of an polypeptide sequence which has one or more of the aforementioned changes to the reference sequence but still retains full or part of the functions of the reference sequence, for example, at least 75%, at least 80%, at least 85%, at least 87%, at least 90%, at least 91%, at least 92%, at least 93%, at least 94%, at least 95%, at least 96%, at least 97%, at least 98%, at least 99%, or 100% of the functions of the reference sequence.

By way of example, various codon optimization techniques can be used to obtain an optimized amino acid sequence from the IL-18 polypeptide, CAR, or other polypeptides discussed herein. For example, it may be desirable to improve the binding affinity and/or other biological properties of an antigen biding domain or other moieties. Amino acid sequence variants may be prepared by introducing appropriate modifications into the nucleotide sequence encoding a polypeptide, or by peptide synthesis. Such modifications include, for example, deletions from, and/or insertions into and/or substitutions of residues within an amino acid sequence. Any combination of deletion, insertion, and substitution can be made to arrive at the final construct, provided that the final construct possesses the desired characteristics, e.g., antigen-binding.

In some embodiments, antibody binding domain moieties or other polypeptide moieties comprising one or more amino acid substitutions, deletions, or insertions are contemplated. Sites of interest for mutational changes include the antibody binding domain heavy and light chain variable regions (VRs) and frameworks (FRs). Amino acid substitutions may be introduced into a binding domain of interest and the products screened for a desired activity, e.g., retained/improved antigen binding or decreased immunogenicity. In certain embodiments, amino acid substitutions may be introduced into one or more of the primary co-stimulatory receptor domain (extracellular or intracellular), secondary costimulatory receptor domain, or extracellular co-receptor domain.

Accordingly, the invention encompasses the polypeptides particularly disclosed herein as well as polypeptides having at least 75%, at least 80%, at least 85%, at least 87%, at least 90%, at least 91%, at least 92%, at least 93%, at least 94%, at least 95%, at least 96%, at least 97%, at least 98%, at least 99% sequence identity to the amino acid sequences particularly disclosed herein. The terms “percent similarity,” “percent identity,” and “percent homology” when referring to a particular sequence are used as set forth in the University of Wisconsin GCG software program BestFit. Other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448),

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, 5, 10, 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:

Conservative substitutions are shown in the Table below.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucinne; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser(S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties: a. hydrophobic: Norleucine, Met, Ala, Val, Leu, Ile; b. neutral hydrophilic: Cys, Ser, Thr, Asn, Gin; c. acidic: Asp, Glu; d. basic: His, Lys, Arg; e. residues that influence chain orientation: Gly, Pro; aomatic: Trp, Tyr, Phe. Non-conservative substitutions will entail exchanging a member of one of these classes for another class.

Vectors

Vectors may be used to introduce the nucleic acid sequence(s) or nucleic acid construct(s) into a host cell so that it expresses one or more CAR, TCR or antigen recognition domain fused to CD3 chain of TCR complex, and cytokine (namely, IL-18) according to an aspect of the invention and, optionally, one or more other proteins of interest (POI). The vector may, for example, be a plasmid or a viral vector, such as a retroviral vector or a lentiviral vector, or a transposon-based vector or synthetic mRNA.

Vectors derived from retroviruses, such as the lentivirus, are suitable tools to achieve long-term gene transfer since they allow long-term, stable integration of a transgene or transgenes and its propagation in daughter cells. The vector may be capable of transfecting or transducing a lymphocyte.

In some embodiments, a nucleic acid discussed in the present disclosure is inserted into a vector. Two nucleic acids can be inserted into one vector or two separate vectors. The expression of natural or synthetic nucleic acids encoding a TCR, CAR or antigen recognition domain fused to CD3 chain of TCR complex and constitutive or inducible cytokine can be achieved by operably linking a nucleic acid encoding the CAR, TCR or antigen recognition domain fused to CD3 chain of TCR complex polypeptide or portions thereof to one promoters and the cytokine expressing portion to another promoter, and incorporating the construct into an expression vector. Another way to achieve such expression is to put the two nucleic acids under the control of one promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

The vectors can be suitable for replication and integration in eukaryotic cells. Typical cloning vectors contain transcription and translation terminators, initiation sequences, and promoters useful for regulation of the expression of the desired nucleic acid sequence. Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193). In some embodiments, the nucleic acid construct of the invention is a multi-cistronic construct comprising two promoters; one promoter driving the expression of the TCR or CAR. In some embodiments, the dual promoter constructs of the invention are uni-directional. In other embodiments, the dual promoter constructs of the invention are bi-directional. In order to assess the expression of the CAR or TCR polypeptide and cytokine polypeptides, the expression vector to be introduced into a cell can also contain either a selectable marker gene or a reporter gene or both to facilitate identification and selection of expressing cells from the population of cells sought to be transfected or transduced through viral vectors.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying chimeric receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly(lactic-co-glycolic acid) (PLGA) and poly lactic acid (PLA), poly(ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the chimeric receptor-encoding nucleic acids to the engineered immune cells. See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, nucleic acids are introduced to the engineered immune cells by a physical method, including, but not limited to electroporation, sonoporation, photoporation, magnetofection, hydroporation.

Cells

The immunoresponsive cells used in the present invention comprise γδ T cells. They can be allogeneic or autologous.

In certain embodiments, therapeutic cells of the invention comprise autologous cells engineered to express a construct of the invention. In certain embodiments, therapeutic cells of the invention comprise allogeneic cells engineered to express a construct of the invention. Autologous cells may be advantageous in avoiding graft-versus-host disease (GVHD) due to CAR-or TCR-mediated recognition of recipient alloantigens. Also, the immune system of a recipient could attack the infused CAR- or TCR-bearing cells, causing rejection. In certain embodiments, to prevent GVHD, and to reduce rejection, endogenous TCR is removed from allogeneic cells by genome editing.

γδ T Cells

γδ T cells are a subgroup of T cells with distinct T cell receptors (TCRs) γ and δ chains on their surface. γδ T cells are a group of heterogeneous T cells, composed of a variety of subgroups, based on their TCRs composition and cellular function. Based on the TCR structure, human γδ T cells can be divided into four main populations based on TCR S chain expression (61, 62, 63, 65). Furthermore, the different TCR δ chains and TCR γ chains combine together to form different γδ T cell types. For example, γδ T cells expressing a TCR containing γ-chain variable region 9 (Vγ9) and δ-chain variable region 2 (Vδ2), are referred to as Vγ9 V82 T cells. In both humans and mice, Vγ2, Vγ3, Vγ4, Vγ5, Vγ8, Vγ9, and Vγ11 rearrangements of the γ chain are found.

All classes of γδ T cells are contemplated in the present disclosure and can be suitably used to carry out the present invention. In an embodiment, the engineered γδ T cell of the invention is selected from the group consisting of γ9δ2 T cell, δ1 T cell, δ3 T cell, or the combination thereof.

The present invention, in an aspect, provides a method of making an engineered CAR (or TCR) γδ T cell armored with IL-18, which comprises introducing into a γδ T cell: (i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a fragment thereof, or a chimeric cytokine receptor comprising the endodomain of the IL-18 receptor (IL-18R).

The present invention, in an aspect, provides a kit for making an engineered CAR (or TCR) γδ T cell armored with IL-18, which comprises:
(a) a container comprising
   (1) (i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
   a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
   (ii) a second nucleic acid, which comprises a nucleic acid sequence that encodes an exogenous cytokine IL-18 or a chimeric cytokine receptor comprising the endodomain of the IL-18 receptor (IL-18R);
   or
   (2) a vector comprising the first and second nucleic acids;
(b) a container comprising γδ T cells; and
(c) instructions for using the kit.

Sources of Cells

Prior to expansion and genetic modification, a source of cells (e.g., T cells such as γδ T cells) is cells obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In one aspect, T cells such as γδ T cells are isolated from peripheral blood lymphocytes by lysing the red blood cells and depleting the monocytes, for example, by centrifugation through a PERCOLL™ gradient or by counterflow centrifugal elutriation.

A specific subpopulation of T cells, such as CD3⁺, CD28⁺, CD4⁺, CD8⁺, CD45RA*, and CD45RO⁺ T cells, can be further isolated by positive or negative selection techniques. For example, in one aspect, T cells are isolated by incubation with anti-CD3/anti-CD28-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumour infiltrating lymphocytes (TIL) from tumor tissue or from immunocompromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD16, HLA-DR, and CD8. In certain aspects, it may be desirable to enrich for or positively select for regulatory T cells which typically express CD4⁺, CD25⁺, CD62Lhi, GITR⁺, CD137, PD1, TIM3, LAG-3, CD150 and FoxP3⁺. Alternatively, in certain aspects, T regulatory cells are depleted by anti-CD25 conjugated beads or other similar method of selection.

The methods described herein can include, e.g., selection of a specific subpopulation of immune effector cells, e.g., T cells, that are a T regulatory cell-depleted population, CD25⁺ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25⁺ cells.

A specific subpopulation of effector cells that specifically bind to a target antigen can be enriched for by positive selection techniques. For example, in some embodiments, effector cells are enriched for by incubation with target antigen-conjugated beads for a time period sufficient for positive selection of the desired abTCR effector cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of effector cells present at low levels in the heterogeneous cell population, use of longer incubation times, such as 24 hours, can increase cell yield. Longer incubation times may be used to isolate effector cells in any situation where there are few effector cells as compared to other cell types. The skilled artisan would recognize that multiple rounds of selection can also be used in the context of this invention.

T cells for stimulation can also be frozen after a washing step. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

Allogeneic CAR and TCR Effector Cells

In embodiments described herein, the immune effector cell can be an allogeneic immune effector cell, e.g., γδ T cell. For example, the cell can be an allogeneic γδ T cell, e.g., an allogeneic γδ T cell with endogenous T cell receptor (TCR) or allogeneic γδ T cell lacking expression human leukocyte antigen (HLA), e.g., HLA class I and/or HLA class II.

A T cell described herein can be, e.g., engineered such that it does not express a functional HLA on its surface. For example, a cell described herein can be engineered such that cell surface expression HLA, e.g., HLA class 1 and/or HLA class II, is downregulated. In some aspects, downregulation of HLA may be accomplished by reducing or eliminating expression of beta- 2 microglobulin (B2M).

In some embodiments, the cell can lack a functional HLA, e.g., HLA class I and/or HLA class II. Modified cells that lack expression of a functional HLA can be obtained by any suitable means, including a knock out or knock down of one or more subunit of HLA. For example, the T cell can include a knock down of HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. a cell engineered by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class 11, Gal9, adenosine, and TGFR beta. Inhibition of an inhibitory molecule, e.g., by inhibition at the DNA, RNA or protein level, can optimize a CAR-expressing cell performance. In some embodiments, an inhibitory nucleic acid, e.g., a dsRNA, an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to inhibit HLA

In some embodiments, endogenous HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, Gal9, adenosine, and TGFR beta), in a T cell.

Expression of siRNA and shRNAs in immune cells can be achieved using any conventional expression system, e.g., such as a lentiviral expression system. Exemplary shRNAs that downregulate expression of components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773.

CRISPR to inhibit endogenous TCR or HLA

“CRISPR” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCNI), HVEM (TNFRSF14 or CD270), KIR, A2aR, MHC class I, MHC class II, GAL9, adenosine, and TGFR beta).

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

Activation and Expansion of Immune Cells

T cells, e.g. γδ T cells, may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In some embodiments, expansion can be performed using flasks or containers, or gas-permeable containers known by those of skill in the art and can proceed for 7 days, 8 days, 9 days, 10 days, 11 days, 12 days, 13 days, or 14 days, about 7 days to about 14 days, about 8 days to about 14 days, about 9 days to about 14 days, about 10 days to about 14 days, about 11 days to about 14 days, about 12 days to about 14 days, or about 13 days to about 14 days.

In certain embodiments, the expansion can be performed using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UHCT-1 (commercially available from BioLegend, San Diego, Calif., USA). CAR- or TCR-expressing cells can be expanded in vitro by including one or more antigens, including antigenic portions thereof, such as epitope(s), of a cancer, which can be optionally expressed from a vector, such as a human leukocyte antigen A2 (HLA-A2) binding peptide, e.g., 0.3 gM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. CAR or TCR cells may also be rapidly expanded by re-stimulation with the same antigen(s) of the cancer pulsed onto HLA-A2-expressing antigen-presenting cells. Alternatively, the cells can be further stimulated with, e.g., example, irradiated, autologous lymphocytes or with irradiated HLA-A2⁺ allogeneic lymphocytes and IL-2. In some embodiments, the stimulation occurs as part of the expansion. In some embodiments, the expansion occurs in the presence of irradiated, autologous lymphocytes or with irradiated HLA-A2⁺ allogeneic lymphocytes and IL-2.

In certain embodiments, the cell culture medium comprises IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL, or between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In certain embodiments, the cell culture medium comprises OKT3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, about 1 μg/mL or between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT3 antibody.

In certain embodiments, a combination of IL-2, IL-7, IL-15, IL-18 and/or IL-21 are employed as a combination during the expansion. In some embodiments, IL-2, IL-7, IL-15, IL-18 and/or IL-21 as well as any combinations thereof can be included during the expansion. In some embodiments, a combination of IL-2, IL-15, and IL-18 are employed as a combination during the expansion. In some embodiments, IL-2, IL-7, and IL-18 as well as any combinations thereof can be included. In some embodiments, IL-2, IL-15 as well as any combinations thereof can be included. In some embodiments, IL-2, IL-15 as well as any combinations thereof can be included. In some embodiments, IL-2, IL-15 as well as any combinations thereof can be included.

In certain embodiments, the expansion can be conducted in a supplemented cell culture medium comprising IL-2, OKT-3, and antigen-presenting feeder cells.

In certain embodiments, the expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15, or about 500 IU/mL of IL-15 to about 100 IU/mL of IL-15, or about 400 IU/mL of IL-15 to about 100 IU/mL of IL-15 or about 300 IU/mL of IL-15 to about 100 IU/mL of IL-15 or about 200 IU/mL of IL-15, or about 180 IU/mL of IL-15.

In some embodiments, the expansion culture media comprises about 20 IU/mL of IL-18, about 15 IU/mL of IL-18, about 12 IU/mL of IL-18, about 10 IU/mL of IL-18, about 5 IU/mL of IL-18, about 4 IU/mL of IL-18, about 3 IU/mL of IL-18, about 2 IU/mL of IL-18, about 1 IU/mL of IL-18, or about 0.5 IU/mL of IL-18, or about 20 IU/mL of IL-18 to about 0.5 IU/mL of IL-18, or about 15 IU/mL of IL-18 to about 0.5 IU/mL of IL-18, or about 12 IU/mL of IL-18 to about 0.5 IU/mL of IL-18, or about 10 IU/mL of IL-18 to about 0.5 IU/mL of IL-18, or about 5 IU/mL of IL-18 to about 1 IU/mL of IL-18, or about 2 IU/mL of IL-18. In some embodiments, the cell culture medium comprises about 1 IU/mL of IL-18, or about 0.5 IU/mL of IL-18.

In some embodiments, the expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21, or about 20 IU/mL of IL-21 to about 0.5 IU/mL of IL-21, or about 15 IU/mL of IL-21 to about 0.5 IU/mL of IL-21, or about 12 IU/mL of IL-21 to about 0.5 IU/mL of IL-21, or about 10 IU/mL of IL-21 to about 0.5 IU/mL of IL-21, or about 5 IU/mL of IL-21 to about 1 IU/mL of IL-21, or about 2 IU/mL of IL-21. In some embodiments, the cell culture medium comprises about 1 IU/mL of IL-21, or about 0.5 IU/mL of IL-21.

In some embodiments the antigen-presenting feeder cells (APCs) are PBMCs. In an embodiment, the ratio of CAR- or TCR- expressing cells to PBMCs and/or antigen-presenting cells in the expansion is about 1 to 25, about 1 to 50, about 1 to 100, about 1 to 125, about 1 to 150, about 1 to 175, about 1 to 200, about 1to 225, about 1 to 250, about 1to 275, about 1to 300, about 1 to 325, about 1 to 350, about 1 to 375, about 1 to 400, or about 1 to 500, or between 1 to 50 and 1 to 300, or between 1 to 100 and 1 to 200.

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In further aspects of the present invention, the cells are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

Preparation of CAR- and TCR- Expressing Cells of the Invention

Viral- and non-viral-based genetic engineering tools can be used to generate CAR-T cells, resulting in permanent or transient expression of therapeutic genes. Retrovirus-based gene delivery is a mature, well-characterized technology, which has been used to permanently integrate CARs into the host cell genome (Scholler J., e.g. Decade-long safety and function of retroviral-modified chimeric antigen receptor T cells. Sci. Transl. Med. 2012; 4:132ra53; Rosenberg S. A. et al., Gene transfer into humans-immunotherapy of patients with advanced melanoma, using tumor-infiltrating lymphocytes modified by retroviral gene transduction. N. Engl. J. Med. 1990; 323:570-578).

Non-viral DNA transfection methods can also be used. For example, Singh et al describes use of the Sleeping Beauty (SB) transposon system developed to engineer CAR T cells (Singh H., et al., Redirecting specificity of T-cell populations for CD19 using the Sleeping Beauty system. Cancer Res. 2008; 68:2961-2971) and is being used in clinical trials (see e.g., ClinicalTrials.gov: NCT00968760 and NCT01653717). The same technology is applicable to engineer T-cells and the like according to the invention.

Multiple SB enzymes have been used to deliver transgenes. Mit6s describes a hyperactive transposase (SB100X) with approximately 100-fold enhancement in efficiency when compared to the first-generation transposase. SB100X supported 35-50% stable gene transfer in human CD34(+) cells enriched in hematopoietic stem or progenitor cells. (Mátés L. et al., Molecular evolution of a novel hyperactive Sleeping Beauty transposase enables robust stable gene transfer in vertebrates. Nat. Genet. 2009; 41:753-761) and multiple transgenes can be delivered from multicistronic single plasmids (e.g., Thokala R. et al., Redirecting specificity of T cells using the Sleeping Beauty system to express chimeric antigen receptors by mix-and- matching of VL and VH domains targeting CD123⁺ tumors. PLoS ONE. 2016;11:e0159477) or multiple plasmids (e.g., Hurton L. V. et al., Tethered IL-15 augments antitumor activity and promotes a stem-cell memory subset in tumor-specific T cells. Proc. Natl. Acad. Sci. USA. 2016;113:E7788-E7797). Such systems are used with CoStARs of the invention.

Morita et al, describes the piggyBac transposon system to integrate larger transgenes (Morita D. et al., Enhanced expression of anti-CD19 chimeric antigen receptor in piggyBac transposon-engineered T cells. Mol. Ther. Methods Clin. Dev. 2017; 8:131-140) Nakazawa et al. describes use of the system to generate EBV-specific cytotoxic T-cells expressing HER2-specific chimeric antigen receptor (Nakazawa Y et al, PiggyBac-mediated cancer immunotherapy using EBV-specific cytotoxic T-cells expressing HER2-specific chimeric antigen receptor. Mol. Ther. 2011; 19:2133-2143). Manuri et al used the system to generate CD-19 specific T cells (Manuri P.V.R. et al., piggyBac transposon/transposase system to generate CD19-specific T cells for the treatment of B-lineage malignancies. Hum. Gene Ther. 2010; 21:427-437).

Transposon technology is easy and economical. One potential drawback is the longer expansion protocols currently employed may result in T cell differentiation, impaired activity and poor persistence of the infused cells. Monjezi et al describe development minicircle vectors that minimize these difficulties through higher efficiency integrations (Monjezi R. et al., Enhanced CAR T-cell engineering using non-viral Sleeping Beauty transposition from minicircle vectors. Leukemia. 2017; 31:186-194). These transposon technologies can be used in the invention.

Pharmaceutical Compositions

The present invention also relates to a pharmaceutical composition containing an effective amount of the engineered γδ T cell of the invention and a pharmaceutically acceptable excipient. In some embodiments, the pharmaceutical composition comprises a therapeutically effective amount of the engineered γδ T cell of the invention for treating a hematological cancer or solid tumor.

In some embodiments, the pharmaceutical composition provided herein contains the engineered γδ T cell of the invention in an effective amount, i.e. an amount effective for achieving a desired result, such as an effective amount to treat or prevent a specific disease or disorder, i.e. a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

In the case of cancer, the therapeutically effective amount as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent a composition for expressing a CAR or TCR and cytokine herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that improves progression free survival of a patient. In the case of infectious disease, such as viral infection, the therapeutically effective amount of a cell or composition as disclosed herein can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

As used herein, “pharmaceutically acceptable” or “pharmacologically compatible” means a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, Pa. Such compositions will contain a prophylactically or therapeutically effective amount of the active ingredient provided herein, such as in purified form, together with a suitable amount of excipient so as to provide the form for proper administration to the patient. The formulation should suit the mode of administration.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The phannaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

An aspect of the invention provides a population of the engineered γδ T cells of the invention. A suitable population may be produced by a method described herein. The population of the engineered γδ T cells may be for use as a medicament. For example, a population of the engineered γδ T cells as described herein may be used in cancer immunotherapy therapy, for example adoptive T cell therapy.

Other aspects of the invention provide the use of a population of the engineered γδ T cells as described herein for the manufacture of a medicament for the treatment of cancer, and a method of treating cancer may comprise administering a population of the engineered γδ T cells as described herein to an individual in need thereof.

The population of the engineered γδ T cells may be autologous i.e. the engineered γδ T cells were originally obtained from the same individual to whom they are subsequently administered (i.e. the donor and recipient individual are the same). The population of the engineered γδ T cells may be allogeneic i.e. the engineered γδ T cells were originally obtained from a different individual to the individual to whom they are subsequently administered (i.e. the donor and recipient individual are different). The donor and recipient individuals may be HLA matched to avoid GVHD and other undesirable immune effects.

Following administration of the engineered γδ T cells, the recipient individual may exhibit a cell mediated immune response against cancer cells in the recipient individual. This may have a beneficial effect on the cancer condition in the individual.

Cancer conditions may be characterized by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumor may be immunogenic). For example, the cancer cells may be capable of eliciting a systemic immune response in the individual against one or more antigens expressed by the cancer cells. The tumor antigens that elicit the immune response may be specific to cancer cells or may be shared by one or more normal cells in the individual.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

Method of Treatment

The present disclosure, in an aspect, provides a method of providing an anti-tumor immunity in a subject comprising administering to the subject an effective amount of the engineered γδ T cell or the pharmaceutical composition according to the invention.

The present disclosure, in an aspect, provides a method of treating cancer in a subject, the method comprising administering to the subject an effective amount of the engineered γδ T cell or the pharmaceutical composition according to the invention, wherein the engineered γδ T cells treat the cancer.

The present disclosure, in an aspect, provides a method of delaying or preventing metastasis or recurrence of a cancer in a subject, the method comprising administering to the subject an effective amount of the engineered γδ T cell or the pharmaceutical composition according to the invention, wherein the engineered γδ T cells delay or prevent metastasis or recurrence of the cancer.

The present disclosure, in an aspect, provides use of the engineered γδ T cell or the pharmaceutical composition according to the invention, to treat a cancer or an infectious disease in a subject.

γδ T cells expressing the IL-18 cytokine with CAR or TCR of the present invention may be used for the treatment of haematological cancers or solid tumors.

A method for the treatment of disease provided herein relates to the therapeutic use of the engineered γδ T cells of the invention. In this respect, the engineered γδ T cells may be administered to a subject having an existing disease or condition in order to lessen, reduce or improve at least one symptom associated with the disease and/or to slow down, reduce or block the progression of the disease. The method of the invention may cause or promote T-cell mediated killing of cancer cells. The engineered γδ T cells according to the present invention may be administered to a patient with one or more additional therapeutic agents. The one or more additional therapeutic agents can be co-administered to the patient. By “co-administering” is meant administering one or more additional therapeutic agents and the engineered γδ T cells of the present invention sufficiently close in time such that the engineered γδ T cells can enhance the effect of one or more additional therapeutic agents, or vice versa. In this regard, the engineered γδ T cells can be administered first and the one or more additional therapeutic agents can be administered second, or vice versa. Alternatively, the engineered γδ T cells and the one or more additional therapeutic agents can be administered simultaneously. One co-administered therapeutic agent that may be useful is IL-2, as this is currently used in existing cell therapies to boost the activity of administered cells. However, IL-2 treatment is associated with toxicity and tolerability issues.

As mentioned, for administration to a patient, the engineered γδ T cells of the invention can be allogeneic or autologous to the patient. In certain embodiments, allogeneic cells are further genetically modified, for example by gene editing, so as to minimize or prevent GVHD and/or a patient's immune response against the effector cells.

The engineered γδ T cells are used to treat cancers and neoplastic diseases associated with a target antigen. Cancers and neoplastic diseases that may be treated using any of the methods described herein include tumours that are not vascularized, or not yet substantially vascularized, as well as vascularized tumours. The cancers may comprise non-solid tumours (such as hematological tumours, for example, leukemias and lymphomas) or may comprise solid tumours. Types of cancers to be treated with the engineered γδ T cells of the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and pediatric tumours/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumour, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

When “an immunologically effective amount,” “an anti-tumour effective amount,” “a tumour-inhibiting effective amount,” or “a therapeutic amount” is indicated, the precise amount of the compositions of the present invention to be administered can be determined by a physician with consideration of individual differences in age, weight, tumour size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

γδ T cells expressing CARs or TCRs and the IL-18 cytokine for use in the methods of the present invention may either be created ex vivo from a patient's own peripheral blood (autologous), or in the setting of a haematopoietic stem cell transplant from donor peripheral blood (allogenic), or peripheral blood from an unconnected donor (allogenic). Alternatively, the cells may be derived from ex vivo differentiation of inducible progenitor cells or embryonic progenitor cells. In these instances, γδ T cells expressing the IL-18 cytokine with CAR, TCR or antigen recognition domain fused to CD3 chain of TCR complex, can be generated by introducing to the cells DNA or RNA coding for the cytokine and CAR, TCR or antigen recognition domain fused to CD3 chain of TCR complex, by one of many means including transduction with a viral vector, transfection with DNA or RNA.

Combination Therapies

The engineered γδ T cell described herein or the pharmaceutical composition containing the same may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the engineered γδ T cell described herein or the pharmaceutical composition containing the same may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

### Example 1: Plasmid Construction, Virus Preparation, Titer Evaluation

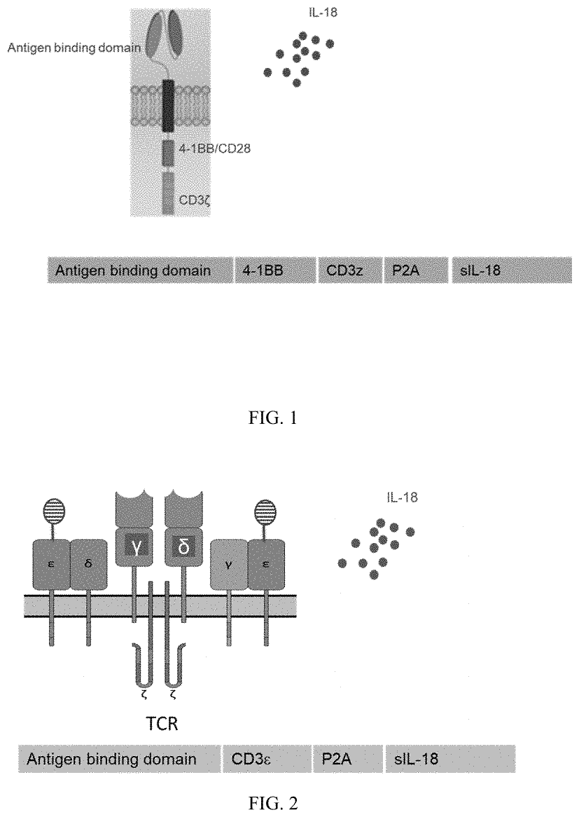

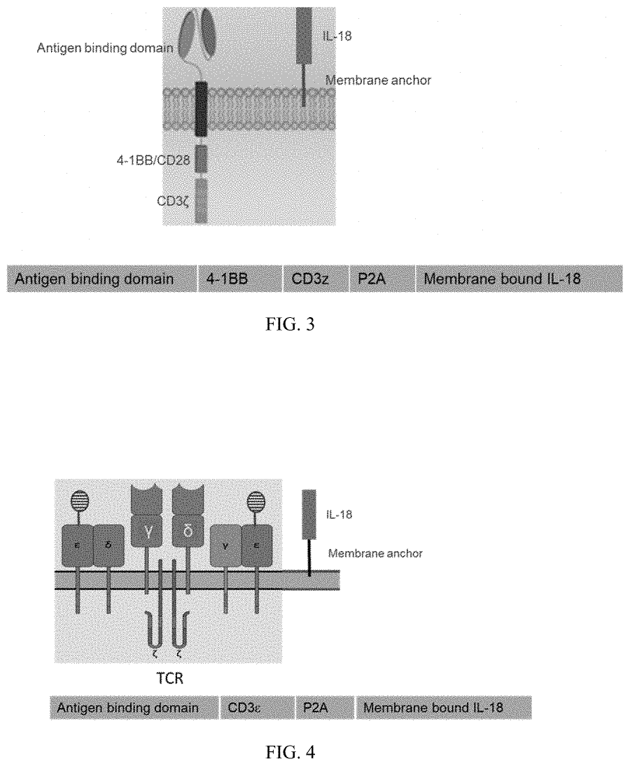

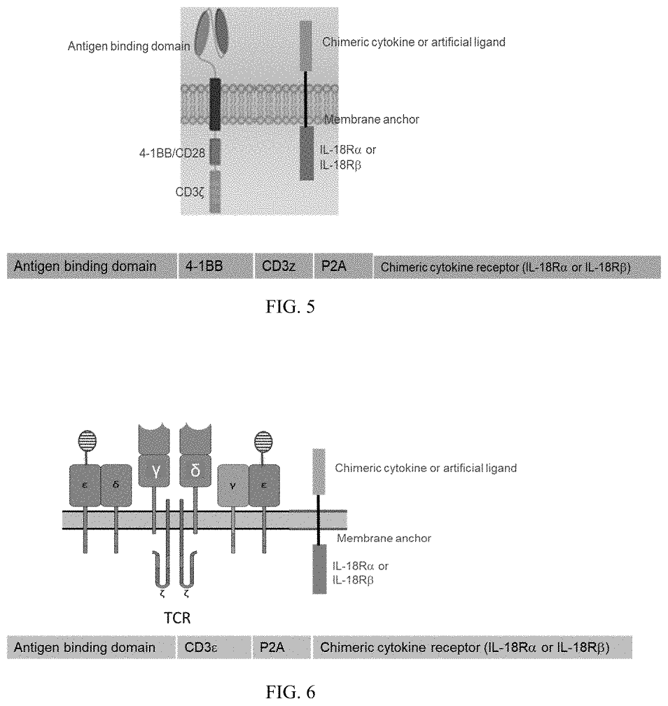

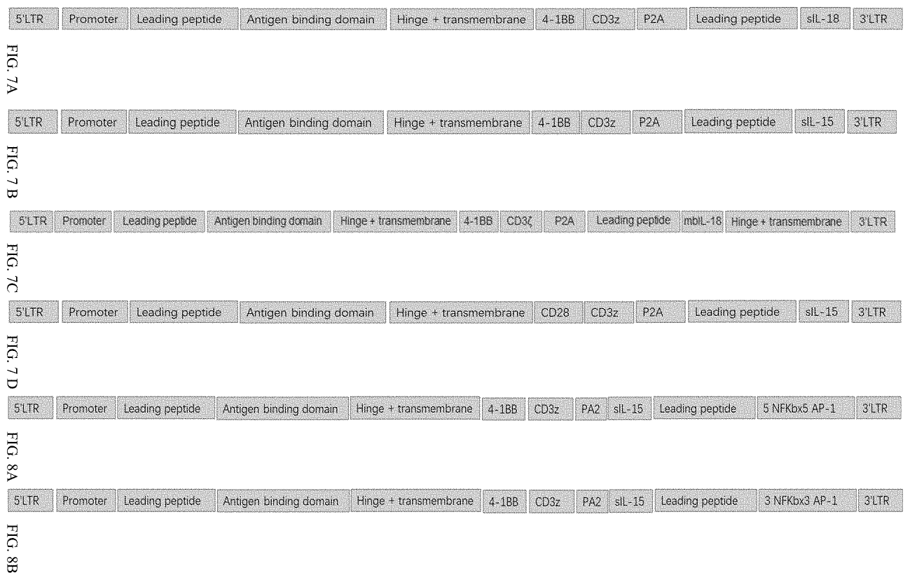

Chimeric antigen receptors armored with different cytokines, were designed as shown in FIGS. 1 to 8 and SEQ ID NO: 1 to SEQ ID NO: 22. To generate viral particles comprising polynucleic acids encoding any of the systems disclosed herein lentivirus packaging plasmid mixture including pMDLg/pRRE (Addgene #11251), pRSV-Rev (Addgene #11253), and pMD2.G (Addgene #11259) were pre-mixed with a PLVX-EF1A (including target system) vector at a pre-optimized ratio with polyetherimide (PEI), mixed properly, and incubated at room temperature for 5 minutes. The transfection mix was added dropwise to 293-T cells and mixed gently. Transfected 293-T cells were incubated overnight at 37° C. and 5% CO₂. Twenty-four hours post-transfection, supernatants were collected and centrifuged at 4° C., 500 g for 10 min to remove any cellular debris. Centrifuged supernatants were filtered through a 0.45 μm PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titers were determined by functional transduction on a T cell line.

Briefly, the lentiviral vector was modified using pLVX-Puro (Clontech #632164) by replacing the original promoter with human elongation factor 1a promoter (hEF1α) and by removing the puromycin resistance gene with EcoR1 and BamHI by GenScript. PLVX-EF1A was further subjected to the lentivirus packaging procedure as described above.

### Example 2: T Cell Transduction and FACS Analysis of Transduced T Cells

γδ T cells were prepared by addition of 5 μM Zoledronate and 1000 IU/mL IL-2 to PBMCs and cultured for 14 days with periodical change of media supplemented with 1000 IU/mL IL-2. Alternatively, γδ T cells were isolated from PBMC or umbilical cord blood (UCB) and then stimulated by anti-γδ TCR antibody and anti-CD3 (OKT3) followed by co-incubation of K562-based artificial antigen-presenting cells (aAPCs) at an 1:2 ratio for at least 10 days.

PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). Forty-eight hours post-activation, cells were transduced with lentiviral vectors encoding the system of Example 1 at an MOI of 5 with 5 pg/ml polybrene. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ⁺ T cell isolation kit (Miltenyi Biotec) before future applications or cryopreserved.

### Example 3: Quantification of Transgene Expression

On day 3 and onwards (typically day 3, 7 and 14) post transduction, cells were evaluated for expression of the system of Example 1 by flow cytometry. An aliquot of cells was collected from the culture before washed, pelleted, and resuspended in diluted antibodies (eBioscience) at a dilution factor of 100 in PBS+0.5% FBS for 50-100 μl per sample. Resuspended cells were resuspended in about 50 to 100 μl of solution. Cell were incubated at 4° C. for 30 minutes. Viability dye eFluor780 or SYTOX Blue viability stain was also added according to manufacturer's instructions. Post-incubation, cells were washed twice in PBS and resuspended in 100 to 200 μl PBS for analysis. The mean fluorescence of the system was quantified by flow cytometry.

For anti-BCMA CAR-T staining, cells were stained with Alexa Fluor 488-labeled mouse-anti-camel sdAb antibodies (Genscript). Flow cytometry analysis for all experiments was performed by using FlowJo (Tree Star, Inc.).

For anti-CD19 CAR-T staining, cells were stained with Alexa Fluor 488-labeled human CD19 protein (Genscript). Flow cytometry analysis for all experiments was performed by using FlowJo (Tree Star, Inc.).

For anti-GPC3 CAR-T staining, cells were stained with Alexa Fluor 488-labeled mouse-anti-human F(ab')2 scFv antibodies (Genscript). Flow cytometry analysis for all experiments was performed by using FlowJo (Tree Star, Inc.).

### Example 4: In Vitro Short-Term Killing and Cytokine Release

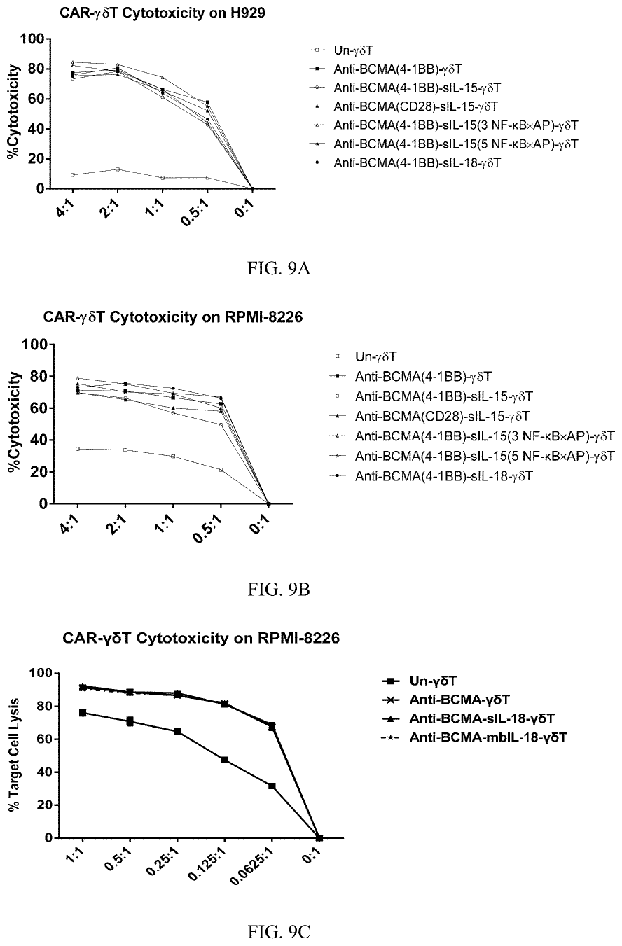

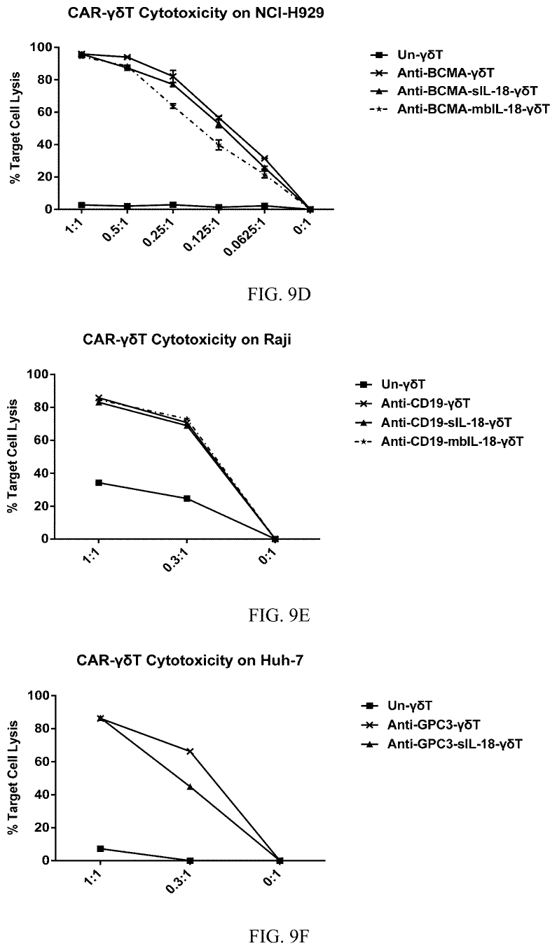

Cells were transduced with lentiviral vectors described in Example 1. Cytotoxic activity was assessed seven days post-transduction. Specifically, transduced or non-transduced γδ T cells were incubated with BCMA or CD19 or GPC3 positive target cell line, H929 or Raji or Huh7, respectively, and the short-tem cytotoxic effect of γδ T cells were evaluated by an LDH assay kit (Roche). Results showed similar short-term in vitro cytotoxicity against multiple myeloma tumor cell lines H929 and RPMI-8226 between IL-18 armored CAR-T and IL-15 armored CAR-T and variants. (FIGS. 9A and 9B). Furthermore, similar short-term cytoicity was found between soluble and membrane-bound IL-18 armored CAR-γδ T cells against BCMA-positive multiple myeloma target cells, RPMI-8226 and NCI-H929 (FIGS. 9C and 9D) as well as CD19-positive B cell malignancies target cells, Raji (FIG. 9E). In addition, similar anti-tumor cytoxicicity were observed between unarmored and soluble IL-18 armored CAR-γδ T cells against GPC-3-positive liver cancer target cell Huh7 (FIG. 9F).

To summarize, no major differences in short-term cytotoxicity was observed for the IL-15 or IL-18 and their variants armored CAR-γδ T cells compared to the unarmored CAR-γδ T cells in three different indications tested. This was expected since the cytokine armors primarily served to extend the persistence of immune cells, such as long-term cytotoxicity or in vivo settings.

The supernatant of the cytotoxicity assay plate was collected for cytokine release analysis (Human IFN gamma kit, Cisbio, Cat #62HIFNGPEH, Human TNF alpha kit, Cisbio, Cat #62HTNFAPEH, Human IL6 kit, Cisbio, Cat #62HIL06PEG, and Human IL2 kit, Cisbio, Cat #62HILO2PEH). The cell supernatant and a standard were dispensed directly into the assay plate for the cytokine detection utilizing HTRF® reagents. The antibodies labeled with the HTRF donor and acceptor were pre-mixed and added in a single dispensing step.

The ELISA standard curve was generated using the 4 Parameter Logistic (4PL) curve. The standard curve regression enables the accurate measurement of an unknown sample concentration across a wider range of concentrations than linear analysis, making it suitable for the analysis of biological systems such as cytokine release. Applicable assay kits included human IFN gamma kit, Cisbio, Cat #62HIFNGPEH; Human TNF alpha kit, Cisbio, Cat #62HTNFAPEH; and Human IL2 kit, Cisbio, Cat #62HILO2PEH. IL-15 kit (R&D, D1500), IL-18 kit (Cat #62HIL-18PEG)

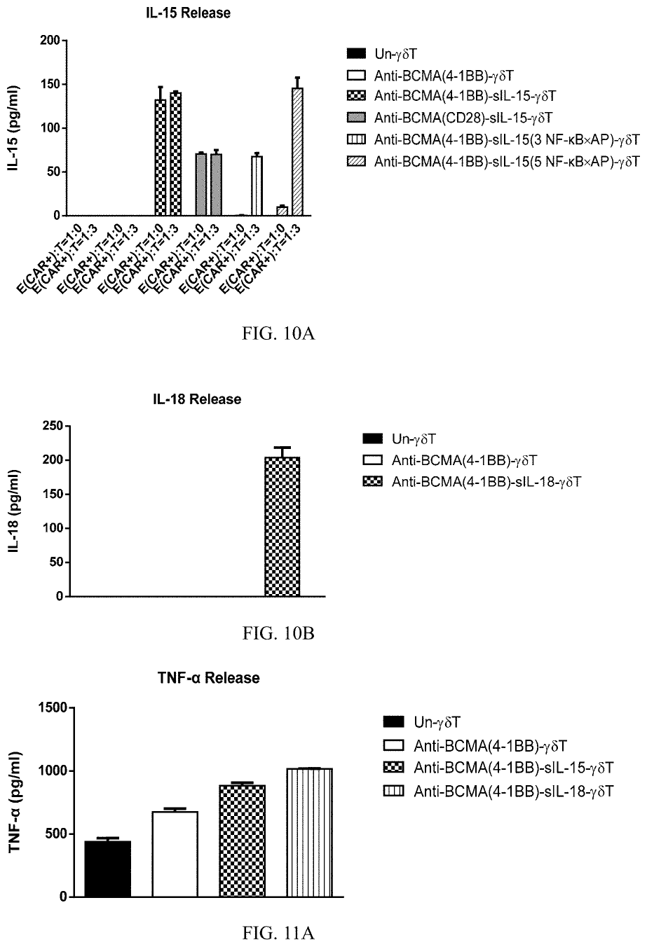

The results showed that IL-15 are expressed at different levels with IL-15 related constructs (FIG. 10A). Notably, constructs under 5 NF-κB×5 AP-1 and 3 NF-κB XAP-I inducible elements displayed activation-inducible IL-15 expression upon engagement with BCMA-positive target cells. IL-18 levels were around 200 pg/mL under our test (FIG. 10B). Further, CARs that constitutively secreting IL-15 and IL-18, alongside unarmored control, showed similar level of TNF-α and GM-CSF secretion. On the other hand, anti-BCMA (4-1BB)-sIL-18-γδT cells showed around two-fold higher IFN-γ secretion than the rest of the constructs tested. This demonstrated the pro-inflammatory effects of IL-18, by which IFN-γ synthesis is upregulated upon IL-18 stimulation in immune cells.

### Example 5: In Vitro Long-Term Cytotoxicity and Persistence

Persistence of CAR-γδ T cells were evaluated with a repetitive tumor challenge assay. In brief, 1×10⁵ CAR⁺ γδ T cells were co-cultured with 3×10⁵ BCMA-positive H929, NCI-RPMI-8226, CD19 positive Raji or GPC-3 positive Huh7 cells in a 24 well. Two days later, cells were harvested to determine the relative ratio of viable T cell and tumor cell. CAR⁺ T cells were quantified and re-plated with fresh H929 cells at a ratio of 1:3 for the next round. IFN-γ release in the supernatant was determined at the end of each round.

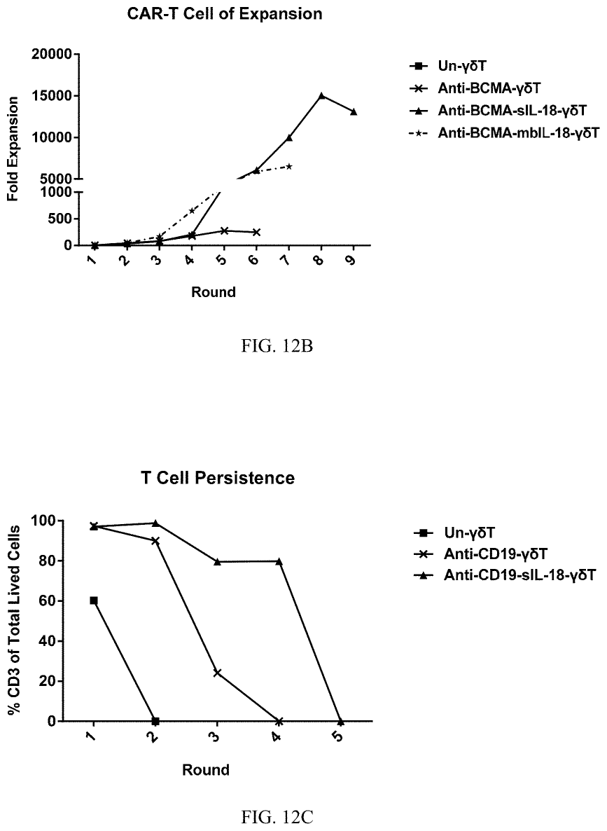

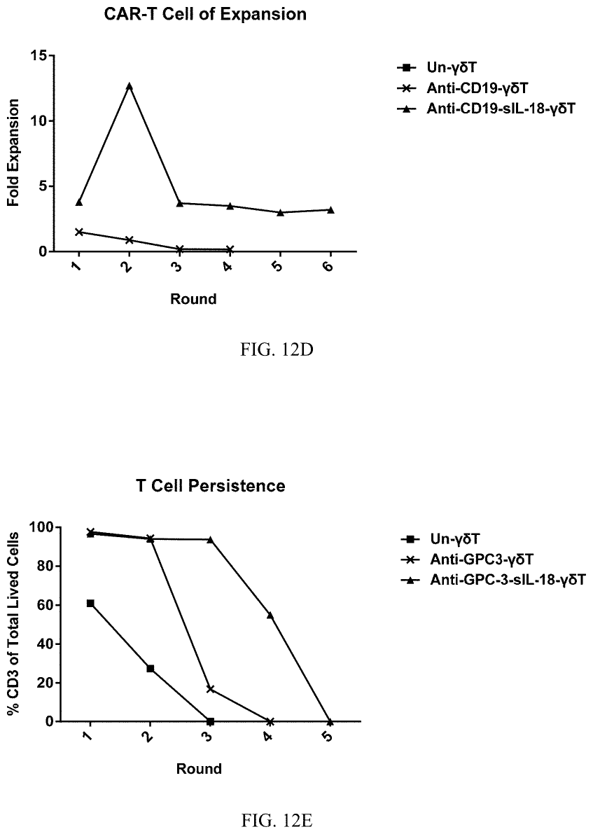

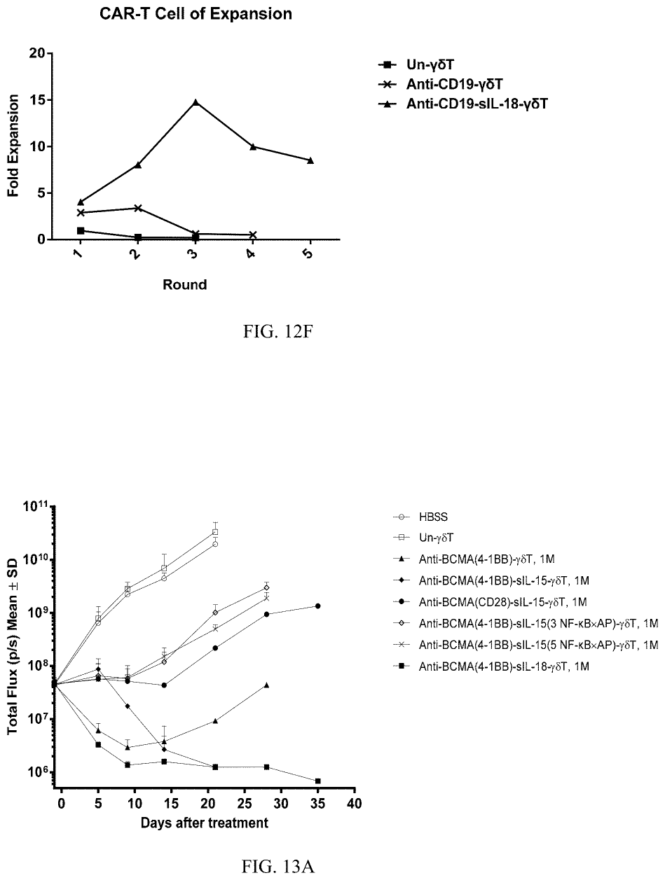

As shown in FIG. 12, both solule IL-18 and membrane-bound IL-18 armored CAR-γδ T cells displayed better persistence in anti-tumor cytotoxicity (FIG. 12A) and expansion (FIG. 12B) compared to unarmored CAR-γδ T cells. It is worth noting that soluble IL-18 armored γδ T cells appeared to have a slightly better anti-tumor cytotoxicicy and expansion than membrane-bound IL-18. In addition, soluble IL-18 armored CAR-γδ T cells were found to outperform unarmored CAR-γδ T cells in anti-tumor cytotoxicity (FIGS. 12C and 12E) and expansion (FIGS. 12D and 12F) against B cell malignancies and liver cancer, respectively.

In summary, IL-18-armord γγ T cells displayed superior persistence and anti-tumor cytoxociciy judged by an in vitro long-term cytotoxicity assay in indications like multiplte myeloma, B cell malignancies and liver cancer.

### Example 6: In vivo safety and efficacy evaluation

Anti-tumor activity of an exemplary anti-BCMA CAR-T was assessed in vivo in an RPMI-8226 xenograft model. Briefly, one million (1×10⁶) RPMI-8226 cells stably expressing the firefly luciferase reporter were implanted subcutaneously/intravenously on day 0 in NOD/SCID IL-2Rγc null (NSG) mice. Fourteen days after tumor inoculation, mice were treated with intravenous injection of 1×10⁶ armored CAR-γδ T or mock T cells or phosphate-buffered saline (PBS). Tumor progression was monitored by bioluminescent imaging (BLI) once a week. In addition, T cell proliferation was monitored via FACS analysis from plasma drawn from blood.

Anti-tumor activity of an exemplary anti-CD19 CAR-T was assessed in vivo in a Raji xenograft model. Briefly, one million (1×10⁶) Raji cells stably expressing the firefly luciferase reporter were implanted subcutaneously/intravenously on day 0 in NOD/SCID IL-2Rγc null (NSG) mice. Seven days after tumor inoculation, mice were treated with intravenous injection of 4×10⁶ armored CAR-γδ T or mock T cells or phosphate-buffered saline (PBS). Tumor progression was monitored by bioluminescent imaging (BLI) once a week. In addition, T cell proliferation was monitored via FACS analysis from plasma drawn from blood.

Anti-tumor activity of an exemplary anti-GPC3 CAR-T was assessed in vivo in a huh7 xenograft model. Briefly, 3 million (3×10⁶) huh7 cells were implanted subcutaneously on day 0 in NOD/SCID IL-2Rγc null (NSG) mice. Ten days after tumor inoculation, mice were treated with intravenous injection of 1×10⁶ armored CAR-γδ T or mock T cells or phosphate-buffered saline (PBS). Tumor dimensions were measured with calipers twice a week, and tumor volumes were calculated using the formula V=½ (length×width²). Mice were euthanized when the mean tumor burden in the control mice reached 2,000 mm³. In addition, T cell proliferation was monitored via FACS analysis from plasma drawn from blood.

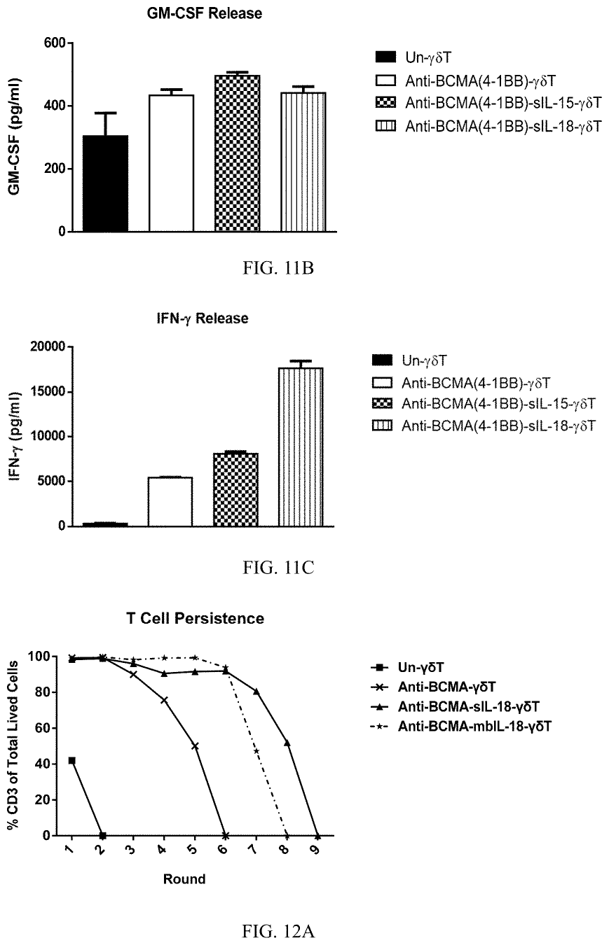

For toxicity evaluations, clinical symptoms were observed every day, while the animals' body weights and the fluorescence intensities triggered by tumor-Luc cells were measured every week. Blood (0.2 mL) was taken every week for detecting the humanized cytokine profiles (IL-15, IL-18 IFN-γ and TNF) in mice (FIG. 11A, FIG. 11B, and FIG. 11C).

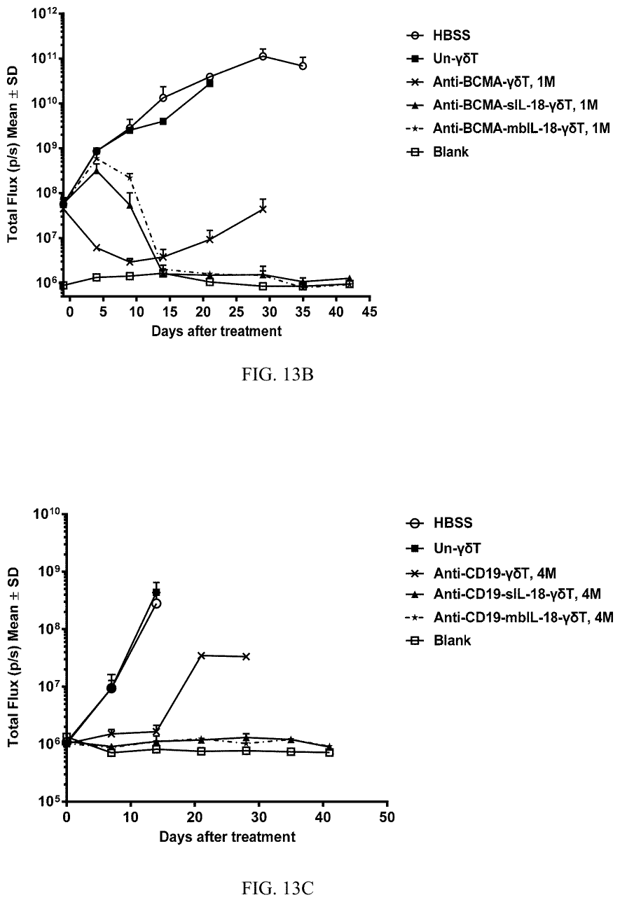

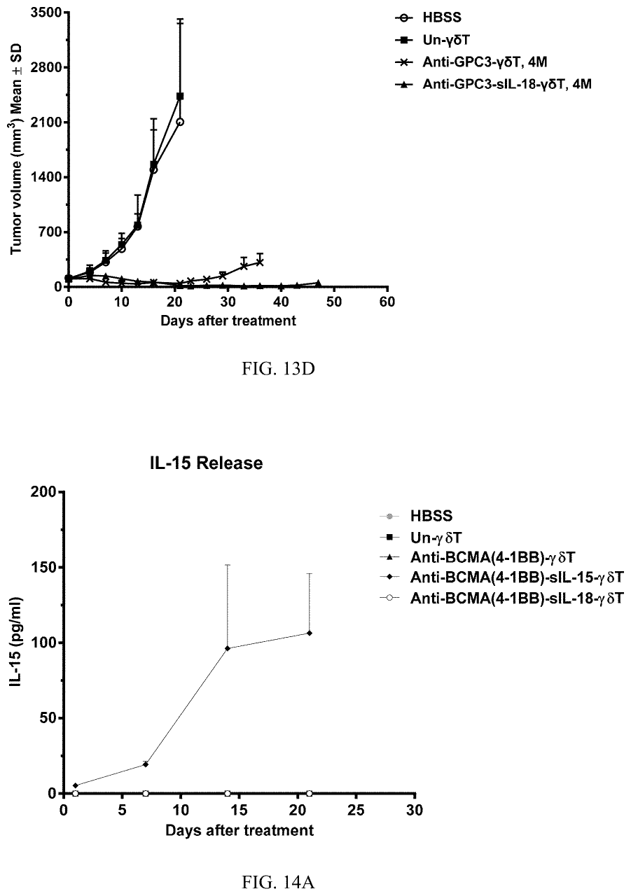

We found that unarmored CAR-γδT, alongside soluble IL-15 and IL-18 armored CAR-γδT can inhibit tumor growth (FIG. 13A). Specifically, unarmored CAR-γδT treated mice reached the lowest tumor burden around Day 10 post-infusion but slowly repulsed. On the other hand, both soluble IL-15 and IL-18 armored CAR-γδT-treated mice reached tumor-free status around Day 9 and Day 22, respectively. However, soluble IL-15 armored CAR-γδT-treated mice died shortly after they reached tumor-free status due to uncontrolled cell proliferation caused by soluble IL-15 armored. In comparison, soluble IL-18 armored CAR-γδT-treated mice not only reached tumor-free status the earliest among all the groups tested. They also remained healthy and tumor-free till the end of experimental observations. It should also be noted, while IL-15 levels can be reduced by displacing CD28 with 4-1BB or under control of activation-inducible elements, they showed poor in vitro efficacy as these mice showed little reduction of tumor burden in the first two weeks of treatment and subsequently dies of high tumor burden due to disease progression.

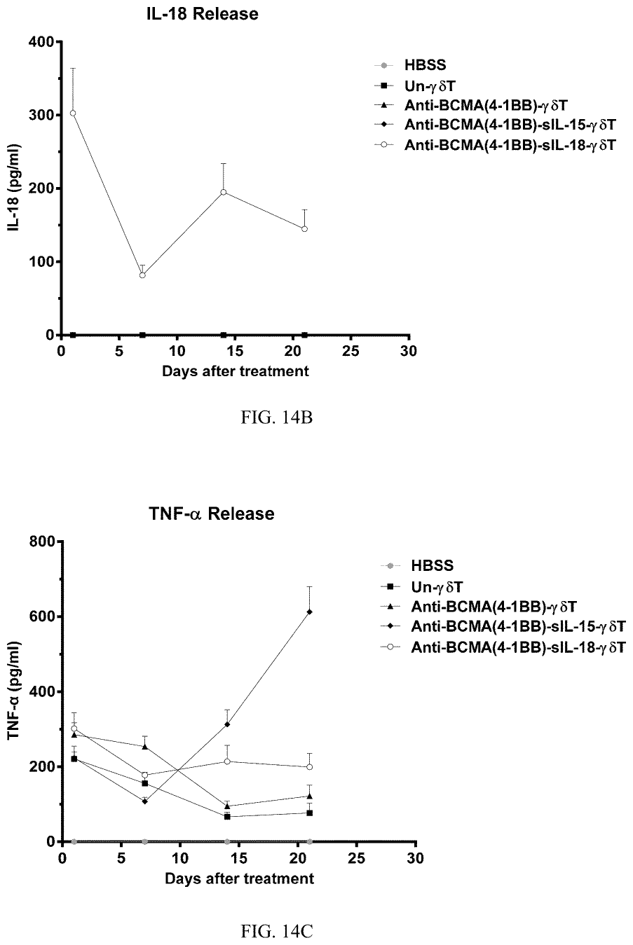

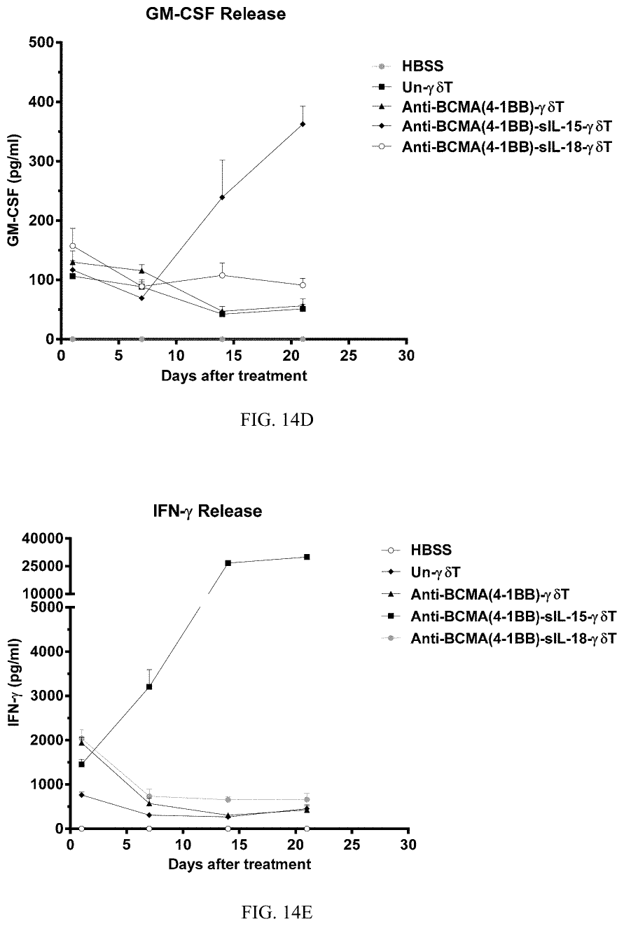

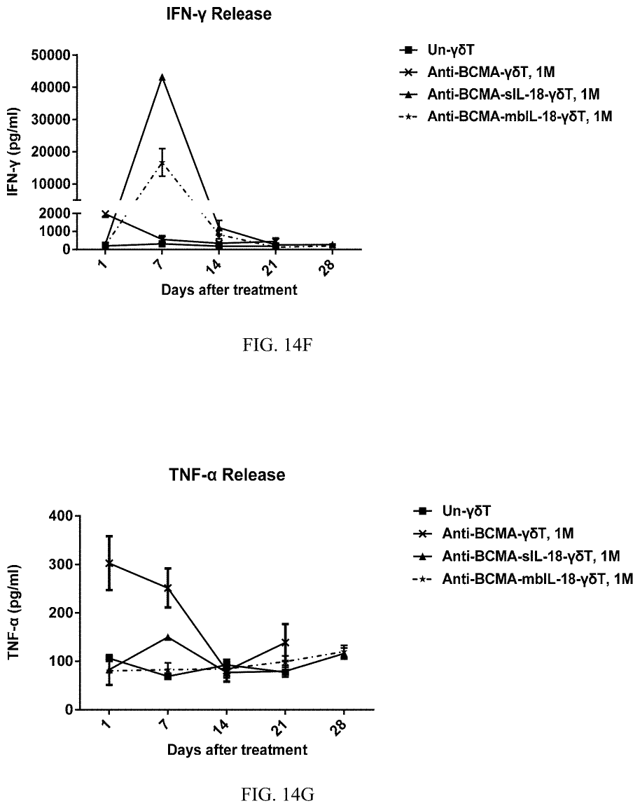

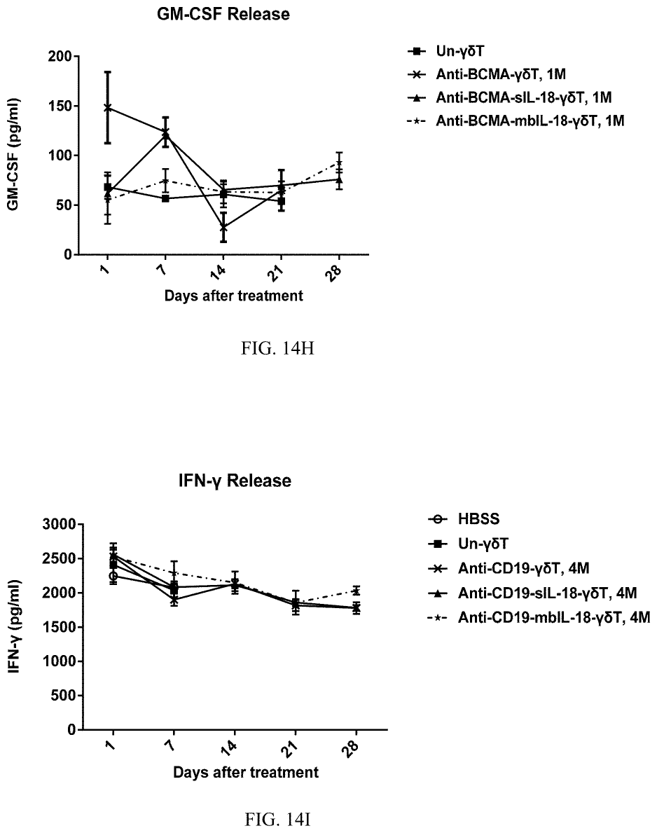

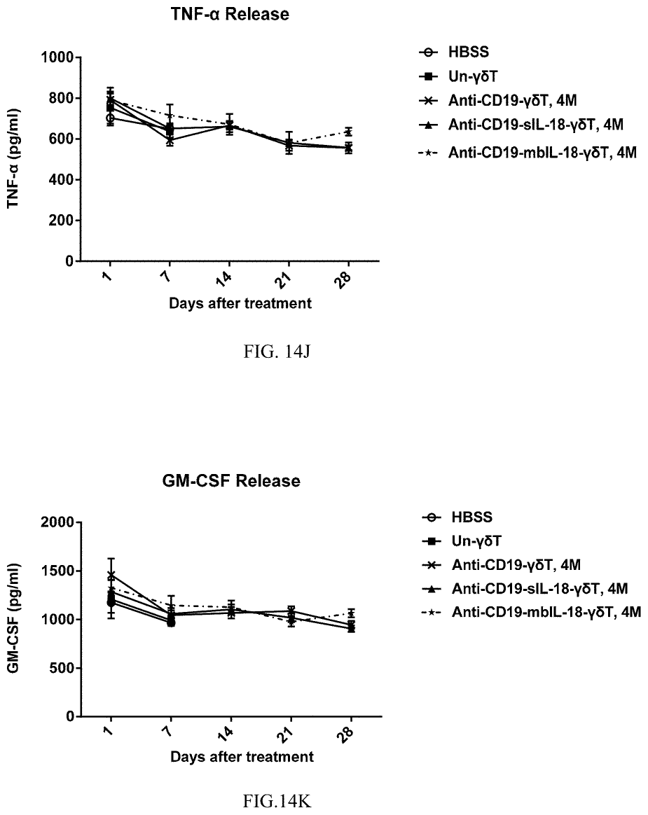

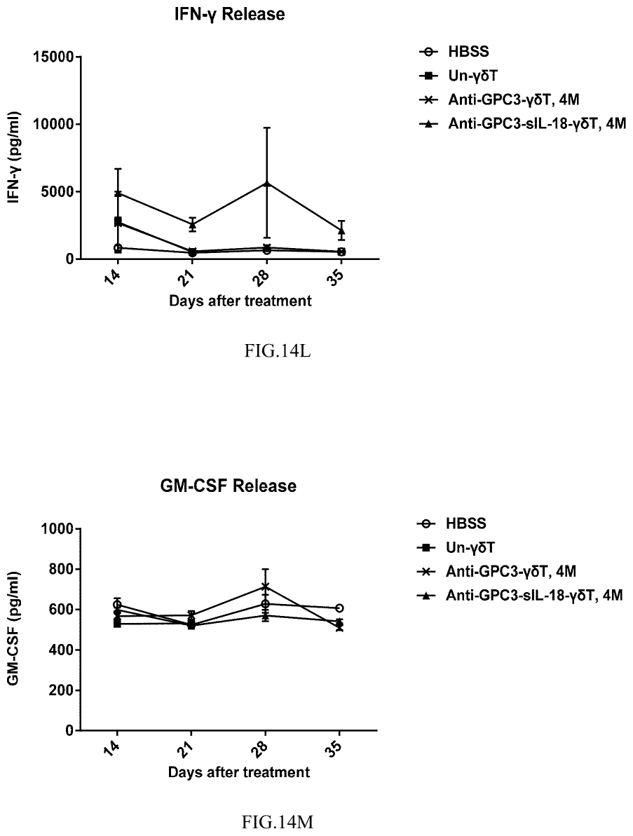

Further, soluble IL-18 armored CAR-γδT-treated mice displayed normal IL-18 (FIG. 14B) level while IL-15 (FIG. 14A) level kept increasing for mice treated with IL-15 armored CAR-γδ T, highlighting unsafe profile of soluble IL-15 armor. In addition, IL-18 armored CAR-γδ T-treated mice displayed a safe cytokine profile with low TNF-α, GM-CSF and IFN-γ secretion detected in the peripheral blood, while soluble IL-15 armored CAR-γδT-treated mice showed ˜20 fold increase over other designs (FIGS. 14C, 14D and 14E), further suggesting the unsafe feature of soluble IL-15 armor.

To summarize, we established in in vivo multiple myeloma model that IL-18 armored anti-BCMA CAR-γδ T cells were efficacious and safe. We next sought to investigate whether there was any difference between solule and membrane-bound form of IL-18 armors in the same animal model.

We found that mice treated with either solule or membrane-bound IL-18 armored CAR—γδ T cells reached tumor-free status around Day 14 and remained tumor-free till the end of observation. In comparison, mice treated with unarmored CAR-γδ T cells never reached tumor-free status and slowly repased after Day 9 post-treatment (FIG. 13B). Further, soluble IL-18 armor appeared to induce more IFN-γ production than membrane-bound IL-18 armor. This demonstrated the superior in vivo efficacy of these armors, TNF-α and GM-CSF production were similarly low among all groups (FIGS. 14F, 14G and 14H), thus demonstrating a safe profile of IL-18 armors in vivo. In conclusion, we found both solule and membrane-bound armored anti-BCMA CAR-γδ T cells efficacious and safe in multiple myeloma animal models.

Next, we sought to investigate if IL-18 armor were applicable to other indications as investigated by in vivo animal models.

In B cell malignancies model, similar to the findings with multiple myeloma models, we found that mice treated with either solule or membrane-bound IL-18 armored CAR-γΥ T cells remained tumor-free till the end of observation. In comparison, mice treated with unarmored CAR-γδ T cells repased after Day 14 post-treatment (FIG. 13B). Interestingly, IFN-γ, TNF-α and GM-CSF production were similarly low among all groups (FIGS. 14I, 14J and 14K), nevertheless demonstrating a safe profile of IL-18 armors in vivo. To conclude, we found both solule and membrane-bound armored anti-CD19 CAR-γδ T cells efficacious and safe in B cell malignancies animal models.

In liver cancer model, we found that mice treated with solule IL-18 armored anti-GPC3-CAR-γδ T cells reached tumor-free status as early as 10 days post-treatment and remained tumor-free till the end of observation. In comparison, mice treated with unarmored CAR-γδ T cells repased after Day 20 post-treatment (FIG. 13D). Further, soluable IL-18 armor appeared to induce more IFN-γ production compared with unarmored control (FIG. 14L). This demonstrated the superior in vivo efficacy and persistence of IL-18 armors, similar to that of multiple myeloma models. GM-CSF production was similarly low among all groups (FIG. 14M), thus demonstrating a safe profile of IL-18 armors in vivo. In conclusion, we found both solule and membrane-bound armored anti-GPC3 CAR-γδ T cells efficacious and safe in liver cancer animal models.

In summary, soluble IL-18 and membrane bound IL-18 armored CAR-γδ T were efficacious and safe in treating multiple myeloma, B cell malignancies and solid tumors such as liver cancer as demonstrated via in vitro efficacy and in vivo efficacy and safety tests.

Sequence Listing

SEQ ID NO: 1 (Anti-BCMA 4-1BB CAR amino acid sequence)
MALPVTALLLPLALLLHAARPAVQLVESGGGLVQAGDSLRLTCTASGRAFSTYFMAWFRQAPG
KEREFVAGIAWSGGSTAYADSVKGRFTISRDNAKNTVYLQMNSLKSEDTAVYYCASRGIEVEEF
GAWGQGTQVTVSSGGGGSQVQLEESGGGSVQAGGSLRLSCAYTYSTYSNYYMGWFREAPGKA
RTSVAIISSDTTITYKDAVKGRFTISKDNAKNTLYLQMNSLKPEDSAMYRCAAWTSDWSVAYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 2 (Anti-BCMA 4-1BB CAR armored with soluble IL-18 amino acid sequence)
MALPVTALLLPLALLLHAARPAVQLVESGGGLVQAGDSLRLTCTASGRAFSTYFMAWFRQAPG
KEREFVAGIAWSGGSTAYADSVKGRFTISRDNAKNTVYLQMNSLKSEDTAVYYCASRGIEVEEF
GAWGQGTQVTVSSGGGGSQVQLEESGGGSVQAGGSLRLSCAYTYSTYSNYYMGWFREAPGKA
RTSVAIISSDTTITYKDAVKGRFTISKDNAKNTLYLQMNSLKPEDSAMYRCAAWTSDWSVAYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGSGATNFSLLKQAGDVEENPG
PMRISKPHLRSISIQCYLCLLLNSHFLTEAGIHVFILGCFSAGLPKTEAYFGKLESKLSVIRNLNDQV
LFIDQGNRPLFEDMTDSDCRDNAPRTIFIISMYKDSQPRGMAVTISVKCEKISTLSCENKIISFKEM
NPPDNIKDTKSDIIFFQRSVPGHDNKMQFESSSYEGYFLACEKERDLFKLILKKEDELGDRSIMFT
VQNED
SEQ ID NO: 3 (Anti-BCMA 4-1BB CAR armored with soluble IL-15 CAR amino acid sequence)
MALPVTALLLPLALLLHAARPAVQLVESGGGLVQAGDSLRLTCTASGRAFSTYFMAWFRQAPG
KEREFVAGIAWSGGSTAYADSVKGRFTISRDNAKNTVYLQMNSLKSEDTAVYYCASRGIEVEEF
GAWGQGTQVTVSSGGGGSQVQLEESGGGSVQAGGSLRLSCAYTYSTYSNYYMGWFREAPGKA
RTSVAIISSDTTITYKDAVKGRFTISKDNAKNTLYLQMNSLKPEDSAMYRCAAWTSDWSVAYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGSGATNFSLLKQAGDVEENPG
PMRISKPHLRSISIQCYLCLLLNSHFLTEAGIHVFILGCFSAGLPKTEANWVNVISDLKKIEDLIQSM
HIDATLYTESDVHPSCKVTAMKCFLLELQVISLESGDASIHDTVENLIILANNSLSSNGNVTESGC
KECEELEEKNIKEFLQSFVHIVQMFINTS
SEQ ID NO: 4 (Anti-BCMA CD28 CAR armored with soluble IL-15 amino acid sequence)
MALPVTALLLPLALLLHAARPAVQLVESGGGLVQAGDSLRLTCTASGRAFSTYFMAWFRQAPG
KEREFVAGIAWSGGSTAYADSVKGRFTISRDNAKNTVYLQMNSLKSEDTAVYYCASRGIEVEEF
GAWGQGTQVTVSSGGGGSQVQLEESGGGSVQAGGSLRLSCAYTYSTYSNYYMGWFREAPGKA
RTSVAIISSDTTITYKDAVKGRFTISKDNAKNTLYLQMNSLKPEDSAMYRCAAWTSDWSVAYW
GQGTQVTVSSTSTTTPAPRPPTPAPTIASQPLSLRPEACRPAAGGAVHTRGLDFACDIYIWAPLAG
TCGVLLLSLV1TLYCRSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGSGATNFSLLKQAGDVEENPG
PMRISKPHLRSISIQCYLCLLLNSHFLTEAGIHVFILGCFSAGLPKTEANWVNVISDLKKIEDLIQSM
HIDATLYTESDVHPSCKVTAMKCFLLELQVISLESGDASIHDTVENL1ILANNSLSSNGNVTESGC
KECEELEEKNIKEFLQSFVHIVQMFINTS
SEQ ID NO: 5 (Anti-BCMA 4-1BB CAR armored with membrane-bound IL-18 amino acid sequence)
MALPVTALLLPLALLLHAARPAVQLVESGGGLVQAGDSLRLTCTASGRAFSTYFMAWFRQAPG
KEREFVAGIAWSGGSTAYADSVKGRFTISRDNAKNTVYLQMNSLKSEDTAVYYCASRGIEVEEF
GAWGQGTQVTVSSGGGGSQVQLEESGGGSVQAGGSLRLSCAYTYSTYSNYYMGWFREAPGKA
RTSVAIISSDTTITYKDAVKGRFTISKDNAKNTLYLQMNSLKPEDSAMYRCAAWTSDWSVAYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGSGATNFSLLKQAGDVEENPG
PMRISKPHLRSISIQCYLCLLLNSHFLTEAGIHVFILGCFSAGLPKTEAYFGKLESKLSVIRNLNDQV
LFIDQGNRPLFEDMTDSDCRDNAPRTIFIISMYKDSQPRGMAVTISVKCEKISTLSCENKIISFKEM
NPPDNIKDTKSDIIFFQRSVPGHDNKMQFESSSYEGYFLACEKERDLFKLILKKEDELGDRSIMFT
VQNEDPTNGPKIPSIATGMVGALLLLLVVALGIGLFMRR
SEQ ID NO: 6 (Anti-CD19 4-1BB CAR amino acid sequence)
MALPVTALLLPLALLLHAARPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGGSYAMDYWGQGTSV
TVSSTTTPAPRPPTPAPTIASQPLSLRPEACRPAAGGAVHTRGLDFACDIYIWAPLAGTCGVLLLSL
VITLYCKRGRKKLLYIFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 7 (Anti-CD19 4-1BB CAR armored with soluble IL-18 amino acid sequence)
MALPVTALLLPLALLLHAARPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGGSYAMDYWGQGTSVT
VSSTTTPAPRPPTPAPTIASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGPMRISKPHLRSISI
QCYLCLLLNSHFLTEAG1HVFILGCFSAGLPKTEAYFGKLESKLSVIRNLNDQVLF1DQGNRPLFED
MTDSDCRDNAPRTIFIISMYKDSQPRGMAVTISVKCEKISTLSCENKIISFKEMNPPDNIKDTKSDII
FFQRSVPGHDNKMQFESSSYEGYFLACEKERDLFKLILKKEDELGDRSIMFTVQNED
SEQ ID NO: 8 (Anti-CD19 4-1BB CAR armored with membrane-bound IL-18 amino acid sequence)
MALPVTALLLPLALLLHAARPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGGSYAMDYWGQGTSVT
VSSTTTPAPRPPTPAPTIASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGPMRISKPHLRSISI
QCYLCLLLNSHFLTEAGIHVFILGCFSAGLPKTEAYFGKLESKLSVIRNLNDQVLFIDQGNRPLFED
MTDSDCRDNAPRTIFIISMYKDSQPRGMAVTISVKCEKISTLSCENKIISFKEMNPPDNIKDTKSDII
FFQRSVPGHDNKMQFESSSYEGYFLACEKERDLFKLILKKEDELGDRSIMFTVQNEDPTNGPKIPS
IATGMVGALLLLLVVALGIGLFMRR
SEQ ID NO: 9 (Anti-GPC3 4-1BB CAR amino acid sequence)
MALPVTALLLPLALLLHAARPDVVMTQSPLSLPVTPGEPASISCRSSQSLVHSNANTYLHWYLQK
PGQSPQLLIYKVSNRFSGVPDRFSGSGSGTDFTLKISRVEAEDVGVYYCSQNTHVPPTFGQGTKLE
IKRGGGGSGGGGSGGGGSQVQLVQSGAEVKKPGASVKVSCKASGYTFTDYEMHWVRQAPGQG
LEWMGALDPKTGDTAYSQKFKGRVTLTADESTSTAYMELSSLRSEDTAVYYCTRFYSYTYWGQG
TLVTVSSTTTPAPRPPTPAPTIASQPLSLRPEACRPAAGGAVHTRGLDFACDIYIWAPLAGTCGVLL
LSLVITLYCKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 10 (Anti-GPC3 4-1BB CAR armored with soluble IL-18 amino acid sequence)
MALPVTALLLPLALLLHAARPDVVMTQSPLSLPVTPGEPASISCRSSQSLVHSNANTYLHWYLQK
PGQSPQLLIYKVSNRFSGVPDRFSGSGSGTDFTLKISRVEAEDVGVYYCSQNTHVPPTFGQGTKLE
IKRGGGGSGGGGSGGGGSQVQLVQSGAEVKKPGASVKVSCKASGYTFTDYEMHWVRQAPGQG
LEWMGALDPKTGDTAYSQKFKGRVTLTADESTSTAYMELSSLRSEDTAVYYCTRFYSYTYWGQG
TLVTVSSTTTPAPRPPTPAPTIASQPLSLRPEACRPAAGGAVHTRGLDFACDIYIWAPLAGTCGVLL
LSLVITLYCKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEIGMK
GERRRGKGHDGLYQGLSTATKDTYDALHMQALPPRGSGATNFSLLKQAGDVEENPGPMRISKPH
LRSISIQCYLCLLLNSHFLTEAGIHVFILGCFSAGLPKTEAYFGKLESKLSVIRNLNDQVLFIDQGN
RPLFEDMTDSDCRDNAPRTIFIISMYKDSQPRGMAVTISVKCEKISTLSCENKIISFKEMNPPDNIK
DTKSDIIFFQRSVPGHDNKMQFESSSYEGYFLACEKERDLFKLILKKEDELGDRSIMFTVQNED
SEQ ID NO: 11 (Anti-BCMA 4-1BB CAR armored with 5xNFkb 5xAP-inducible IL-15 nucleic
acid sequence)
ATGGCACTGCCAGTGACAGCACTGCTGCTGCCTCTGGCACTGCTGCTGCACGCAGCAAGGCC
TGCCGTGCAGCTGGTGGAGTCCGGCGGCGGCCTGGTGCAGGCCGGCGACTCTCTGAGACTG
ACATGCACCGCCTCCGGCAGGGCCTTCTCTACATACTTTATGGCCTGGTTCAGACAGGCCCC
AGGCAAGGAGAGGGAGTTTGTGGCAGGAATCGCATGGTCTGGAGGAAGCACCGCATACGCA
GACTCTGTGAAGGGCCGCTTCACAATCAGCCGGGATAACGCCAAGAATACCGTGTATCTGCA
GATGAACTCCCTGAAGTCTGAGGATACCGCCGTGTACTATTGCGCCTCCAGAGGCATCGAGG
TGGAGGAGTTTGGAGCATGGGGACAGGGAACACAGGTGACCGTGAGCTCCGGAGGAGGAG
GATCTCAGGTGCAGCTGGAGGAGTCCGGAGGAGGATCTGTGCAGGCAGGAGGCAGCCTGAG
GCTGTCCTGTGCCTACACATATAGCACCTACTCCAACTACTATATGGGATGGTTTAGGGAGG
CACCAGGCAAGGCCCGGACATCTGTGGCCATCATCTCTAGCGACACCACAATCACCTACAAG
GATGCCGTGAAGGGCAGATTCACAATCAGCAAGGACAACGCCAAGAATACCCTGTATCTGC
AGATGAATAGCCTGAAGCCTGAGGACTCCGCCATGTACAGGTGCGCCGCCTGGACATCTGAT
TGGAGCGTGGCCTATTGGGGCCAGGGCACACAGGTGACCGTGTCCTCTACCAGCACCACAA
CCCCTGCACCAAGGCCACCTACACCAGCACCTACCATCGCCTCTCAGCCTCTGAGCCTGAGA
CCAGAGGCCTGTAGGCCAGCAGCAGGAGGAGCAGTGCACACCCGGGGCCTGGACTTCGCCT
GCGATATCTACATCTGGGCACCACTGGCAGGAACATGTGGAGTGCTGCTGCTGAGCCTGGTC
ATCACCCTGTACTGCAAGAGAGGCAGGAAGAAGCTGCTGTATATCTTTAAGCAGCCATTCAT
GCGCCCCGTGCAGACAACCCAGGAGGAGGACGGCTGCTCCTGTCGGTTTCCAGAAGAGGAG
GAGGGAGGATGTGAGCTGAGGGTGAAGTTCAGCAGGTCCGCAGATGCACCAGCATACCAGC
AGGGCCAGAATCAGCTGTATAACGAGCTGAATCTGGGCCGGAGAGAGGAGTATGACGTGCT
GGATAAGAGGAGGGGAAGGGATCCAGAGATGGGAGGCAAGCCTCGGAGAAAGAACCCACA
GGAGGGCCTGTACAATGAGCTGCAGAAGGACAAGATGGCCGAGGCCTATAGCGAGATCGGC
ATGAAGGGAGAGAGGAGGAGGGGCAAGGGACACGATGGCCTGTACCAGGGCCTGTCCACA
GCCACCAAGGACACCTATGATGCCCTGCACATGCAGGCCCTGCCACCCCGGTAACTCACACA
AAAAACCAACACACAGATGTAATGAAAATAAAGATATTTTATTTTAGGATGTATTGATGAAC
ATCTGCACGATGTGCACAAAGCTCTGCAGGAACTCCTTGATGTTCTTCTCCTCCAGCTCCTCA
CACTCCTTGCAGCCGCTCTCTGTCACATTGCCGTTGGAGCTCAGAGAATTGTTGGCCAGGAT
GATCAGATTCTCCACGGTATCGTGGATAGAGGCGTCGCCAGACTCCAGGGAGATGACCTGC
AGCTCCAGCAGAAAACACTTCATGGCGGTCACCTTGCAGGAAGGGTGCACATCGCTCTCTGT
ATACAGGGTGGCGTCGATGTGCATAGACTGGATCAGATCCTCGATCTTCTTCAGGTCGGAGA
TCACATTCACCCAGTTGGCCTCTGTCTTGGGCAGGCCGGCGCTAAAGCAGCCCAGGATGAAC
ACGTGGATGCCGGCCTCGGTCAGGAAGTGAGAGTTCAGCAGCAGACACAGGTAGCACTGGA
TGCTGATAGATCTCAGGTGGGGCTTGGAGATCCGCATGGCTCTGTCTCAGGTCAGTATAGAA
GCTTTGATGTGAAGTCAGCCAAGAACAGCTGAACACTACTTCTGCTGAGGCCCTTTTATAGG
AGGGATTGCTTCCTGTGAATAATAGGAGGATATTGTCCACATCCAGTAAAGAGGAAATCCCC
AACTGCATCCAAAAAGTTTTCTGGGAATATCCACTGCTGCAGGTGACTCACTGAGTCAGTGA
CTCAAGTGGAAAGTCCCCAGTGGAAAGTCCCCAGTGGAAAGTCCCCAGTGGAAAGTCCCCA
GTGGAAAGTCCCC
SEQ ID NO: 12 (Anti-BCMA 4-1BB CAR armored with 3xNFkb 3xAP-inducible IL-15 nucleic
acid sequence)
ATGGCACTGCCAGTGACAGCACTGCTGCTGCCTCTGGCACTGCTGCTGCACGCAGCAAGGCC
TGCCGTGCAGCTGGTGGAGTCCGGCGGCGGCCTGGTGCAGGCCGGCGACTCTCTGAGACTG
ACATGCACCGCCTCCGGCAGGGCCTTCTCTACATACTTTATGGCCTGGTTCAGACAGGCCCC
AGGCAAGGAGAGGGAGTTTGTGGCAGGAATCGCATGGTCTGGAGGAAGCACCGCATACGCA
GACTCTGTGAAGGGCCGCTTCACAATCAGCCGGGATAACGCCAAGAATACCGTGTATCTGCA
GATGAACTCCCTGAAGTCTGAGGATACCGCCGTGTACTATTGCGCCTCCAGAGGCATCGAGG
TGGAGGAGTTTGGAGCATGGGGACAGGGAACACAGGTGACCGTGAGCTCCGGAGGAGGAG
GATCTCAGGTGCAGCTGGAGGAGTCCGGAGGAGGATCTGTGCAGGCAGGAGGCAGCCTGAG
GCTGTCCTGTGCCTACACATATAGCACCTACTCCAACTACTATATGGGATGGTTTAGGGAGG
CACCAGGCAAGGCCCGGACATCTGTGGCCATCATCTCTAGCGACACCACAATCACCTACAAG
GATGCCGTGAAGGGCAGATTCACAATCAGCAAGGACAACGCCAAGAATACCCTGTATCTGC
AGATGAATAGCCTGAAGCCTGAGGACTCCGCCATGTACAGGTGCGCCGCCTGGACATCTGAT
TGGAGCGTGGCCTATTGGGGCCAGGGCACACAGGTGACCGTGTCCTCTACCAGCACCACAA
CCCCTGCACCAAGGCCACCTACACCAGCACCTACCATCGCCTCTCAGCCTCTGAGCCTGAGA
CCAGAGGCCTGTAGGCCAGCAGCAGGAGGAGCAGTGCACACCCGGGGCCTGGACTTCGCCT
GCGATATCTACATCTGGGCACCACTGGCAGGAACATGTGGAGTGCTGCTGCTGAGCCTGGTC
ATCACCCTGTACTGCAAGAGAGGCAGGAAGAAGCTGCTGTATATCTTTAAGCAGCCATTCAT
GCGCCCCGTGCAGACAACCCAGGAGGAGGACGGCTGCTCCTGTCGGTTTCCAGAAGAGGAG
GAGGGAGGATGTGAGCTGAGGGTGAAGTTCAGCAGGTCCGCAGATGCACCAGCATACCAGC
AGGGCCAGAATCAGCTGTATAACGAGCTGAATCTGGGCCGGAGAGAGGAGTATGACGTGCT
GGATAAGAGGAGGGGAAGGGATCCAGAGATGGGAGGCAAGCCTCGGAGAAAGAACCCACA
GGAGGGCCTGTACAATGAGCTGCAGAAGGACAAGATGGCCGAGGCCTATAGCGAGATCGGC
ATGAAGGGAGAGAGGAGGAGGGGCAAGGGACACGATGGCCTGTACCAGGGCCTGTCCACA
GCCACCAAGGACACCTATGATGCCCTGCACATGCAGGCCCTGCCACCCCGGTAACTCACACA
AAAAACCAACACACAGATGTAATGAAAATAAAGATATTTTATTTTAGGATGTATTGATGAAC
ATCTGCACGATGTGCACAAAGCTCTGCAGGAACTCCTTGATGTTCTTCTCCTCCAGCTCCTCA
CACTCCTTGCAGCCGCTCTCTGTCACATTGCCGTTGGAGCTCAGAGAATTGTTGGCCAGGAT
GATCAGATTCTCCACGGTATCGTGGATAGAGGCGTCGCCAGACTCCAGGGAGATGACCTGC
AGCTCCAGCAGAAAACACTTCATGGCGGTCACCTTGCAGGAAGGGTGCACATCGCTCTCTGT
ATACAGGGTGGCGTCGATGTGCATAGACTGGATCAGATCCTCGATCTTCTTCAGGTCGGAGA
TCACATTCACCCAGTTGGCCTCTGTCTTGGGCAGGCCGGCGCTAAAGCAGCCCAGGATGAAC
ACGTGGATGCCGGCCTCGGTCAGGAAGTGAGAGTTCAGCAGCAGACACAGGTAGCACTGGA
TGCTGATAGATCTCAGGTGGGGCTTGGAGATCCGCATGGCTCTGTCTCAGGTCAGTATAGAA
GCTTTGATGTGAAGTCAGCCAAGAACAGCTGAACACTACTTCTGCTGAGGCCCTTTTATAGG
AGGGATTGCTTCCTGTGAATAATAGGAGGATATTGTCCACATCCAGTAAAGAGGAAATCCCC
AACTGCATCCAAAAAGTTTTCTGGGAATATCCACTGCTGCAGGTGACTCACTGAGTCAGTGA
CTCAAGTGGAAAGTCCCCAGTGGAAAGTCCCCAGTGGAAAGTCCCC
SEQ ID NO: 13 (Anti-BCMA 4-1BB CAR nucleic acid sequence)
ATGGCCCTGCCCGTGACCGCCCTGCTGCTGCCCCTGGCCCTGCTGCTGCACGCCGCCAGG
CCCGCCGTGCAGCTGGTGGAGAGCGGCGGCGGCCTGGTGCAGGCCGGCGACAGCCTGAGG
CTGACCTGCACCGCCAGCGGCAGGGCCTTCAGCACCTACTTCATGGCCTGGTTCAGGCAG
GCCCCCGGCAAGGAGAGGGAGTTCGTGGCCGGCATCGCCTGGAGCGGCGGCAGCACCGCC
TACGCCGACAGCGTGAAGGGCAGGTTCACCATCAGCAGGGACAACGCCAAGAACACCGTG
TACCTGCAGATGAACAGCCTGAAGAGCGAGGACACCGCCGTGTACTACTGCGCCAGCAGG
GGCATCGAGGTGGAGGAGTTCGGCGCCTGGGGCCAGGGCACCCAGGTGACCGTGAGCAGC
GGCGGCGGCGGCAGCCAGGTGCAGCTGGAGGAGAGCGGCGGCGGCAGCGTGCAGGCCGGC
GGCAGCCTGAGGCTGAGCTGCGCCTACACCTACAGCACCTACAGCAACTACTACATGGGC
TGGTTCAGGGAGGCCCCCGGCAAGGCCAGGACCAGCGTGGCCATCATCAGCAGCGACACC
ACCATCACCTACAAGGACGCCGTGAAGGGCAGGTTCACCATCAGCAAGGACAACGCCAAG
AACACCCTGTACCTGCAGATGAACAGCCTGAAGCCCGAGGACAGCGCCATGTACAGGTGC
GCCGCCTGGACCAGCGACTGGAGCGTGGCCTACTGGGGCCAGGGCACCCAGGTGACCGTG
AGCAGCACCAGCACCACCACCCCCGCCCCCAGGCCCCCCACCCCCGCCCCCACCATCGCC
AGCCAGCCCCTGAGCCTGAGGCCCGAGGCCTGCAGGCCCGCCGCCGGCGGCGCCGTGCAC
ACCAGGGGCCTGGACTTCGCCTGCGACATCTACATCTGGGCCCCCCTGGCCGGCACCTGC
GGCGTGCTGCTGCTGAGCCTGGTGATCACCCTGTACTGCAAGAGGGGCAGGAAGAAGCTG
CTGTACATCTTCAAGCAGCCCTTCATGAGGCCCGTGCAGACCACCCAGGAGGAGGACGGC
TGCAGCTGCAGGTTCCCCGAGGAGGAGGAGGGCGGCTGCGAGCTGAGGGTGAAGTTCAGC
AGGAGCGCCGACGCCCCCGCCTACCAGCAGGGCCAGAACCAGCTGTACAACGAGCTGAAC
CTGGGCAGGAGGGAGGAGTACGACGTGCTGGACAAGAGGAGGGGCAGGGACCCCGAGATG
GGCGGCAAGCCCAGGAGGAAGAACCCCCAGGAGGGCCTGTACAACGAGCTGCAGAAGGAC
AAGATGGCCGAGGCCTACAGCGAGATCGGCATGAAGGGCGAGAGGAGGAGGGGCAAGGGC
CACGACGGCCTGTACCAGGGCCTGAGCACCGCCACCAAGGACACCTACGACGCCCTGCAC
ATGCAGGCCCTGCCCCCCAGG
SEQ ID NO: 14 (Anti-BCMA 4-1BB CAR armored with soluble IL-18 nucleic acid sequence)
ATGGCCCTGCCCGTGACCGCCCTGCTGCTGCCCCTGGCCCTGCTGCTGCACGCCGCCAGG
CCCGCCGTGCAGCTGGTGGAGAGCGGCGGCGGCCTGGTGCAGGCCGGCGACAGCCTGAGG
CTGACCTGCACCGCCAGCGGCAGGGCCTTCAGCACCTACTTCATGGCCTGGTTCAGGCAG
GCCCCCGGCAAGGAGAGGGAGTTCGTGGCCGGCATCGCCTGGAGCGGCGGCAGCACCGCC
TACGCCGACAGCGTGAAGGGCAGGTTCACCATCAGCAGGGACAACGCCAAGAACACCGTG
TACCTGCAGATGAACAGCCTGAAGAGCGAGGACACCGCCGTGTACTACTGCGCCAGCAGG
GGCATCGAGGTGGAGGAGTTCGGCGCCTGGGGCCAGGGCACCCAGGTGACCGTGAGCAGC
GGCGGCGGCGGCAGCCAGGTGCAGCTGGAGGAGAGCGGCGGCGGCAGCGTGCAGGCCGGC
GGCAGCCTGAGGCTGAGCTGCGCCTACACCTACAGCACCTACAGCAACTACTACATGGGC
TGGTTCAGGGAGGCCCCCGGCAAGGCCAGGACCAGCGTGGCCATCATCAGCAGCGACACC
ACCATCACCTACAAGGACGCCGTGAAGGGCAGGTTCACCATCAGCAAGGACAACGCCAAG
AACACCCTGTACCTGCAGATGAACAGCCTGAAGCCCGAGGACAGCGCCATGTACAGGTGC
GCCGCCTGGACCAGCGACTGGAGCGTGGCCTACTGGGGCCAGGGCACCCAGGTGACCGTG
AGCAGCACCAGCACCACCACCCCCGCCCCCAGGCCCCCCACCCCCGCCCCCACCATCGCC
AGCCAGCCCCTGAGCCTGAGGCCCGAGGCCTGCAGGCCCGCCGCCGGCGGCGCCGTGCAC
ACCAGGGGCCTGGACTTCGCCTGCGACATCTACATCTGGGCCCCCCTGGCCGGCACCTGC
GGCGTGCTGCTGCTGAGCCTGGTGATCACCCTGTACTGCAAGAGGGGCAGGAAGAAGCTG
CTGTACATCTTCAAGCAGCCCTTCATGAGGCCCGTGCAGACCACCCAGGAGGAGGACGGC
TGCAGCTGCAGGTTCCCCGAGGAGGAGGAGGGCGGCTGCGAGCTGAGGGTGAAGTTCAGC
AGGAGCGCCGACGCCCCCGCCTACCAGCAGGGCCAGAACCAGCTGTACAACGAGCTGAAC
CTGGGCAGGAGGGAGGAGTACGACGTGCTGGACAAGAGGAGGGGCAGGGACCCCGAGATG
GGCGGCAAGCCCAGGAGGAAGAACCCCCAGGAGGGCCTGTACAACGAGCTGCAGAAGGAC
AAGATGGCCGAGGCCTACAGCGAGATCGGCATGAAGGGCGAGAGGAGGAGGGGCAAGGGC
CACGACGGCCTGTACCAGGGCCTGAGCACCGCCACCAAGGACACCTACGACGCCCTGCAC
ATGCAGGCCCTGCCCCCCAGGGGCAGCGGCGCCACCAACTTCAGCCTGCTGAAGCAGGCC
GGCGACGTGGAGGAGAACCCCGGCCCCATGAGGATCAGCAAGCCCCACCTGAGGAGCATC
AGCATCCAGTGCTACCTGTGCCTGCTGCTGAACAGCCACTTCCTGACCGAGGCCGGCATC
CACGTGTTCATCCTGGGCTGCTTCAGCGCCGGCCTGCCCAAGACCGAGGCCTACTTCGGC
AAGCTGGAGAGCAAGCTGAGCGTGATCAGGAACCTGAACGACCAGGTGCTGTTCATCGAC
CAGGGCAACAGGCCCCTGTTCGAGGACATGACCGACAGCGACTGCAGGGACAACGCCCCC
AGGACCATCTTCATCATCAGCATGTACAAGGACAGCCAGCCCAGGGGCATGGCCGTGACC
ATCAGCGTGAAGTGCGAGAAGATCAGCACCCTGAGCTGCGAGAACAAGATCATCAGCTTC
AAGGAGATGAACCCCCCCGACAACATCAAGGACACCAAGAGCGACATCATCTTCTTCCAG
AGGAGCGTGCCCGGCCACGACAACAAGATGCAGTTCGAGAGCAGCAGCTACGAGGGCTAC
TTCCTGGCCTGCGAGAAGGAGAGGGACCTGTTCAAGCTGATCCTGAAGAAGGAGGACGAG
CTGGGCGACAGGAGCATCATGTTCACCGTGCAGAACGAGGAC
SEQ ID NO: 15 (Anti-BCMA 4-1BB CAR armored with soluble IL-15 CAR nucleic acid sequence)
ATGGCACTGCCTGTCACGGCCCTTCTGCTCCCGCTGGCTCTGCTCCTGCACGCCGCACGTCCA
GCGGTGCAGTTGGTGGAGAGCGGAGGTGGCCTCGTGCAGGCCGGCGATTCTTTGCGGCTGA
CCTGTACAGCATCGGGCCGTGCGTTCTCTACCTATTTCATGGCATGGTTCCGCCAGGCGCCTG
GCAAAGAGCGCGAGTTCGTTGCTGGCATAGCCTGGTCTGGAGGCAGTACCGCTTACGCGGA
CAGCGTGAAGGGCCGGTTCACCATCTCTCGCGACAACGCCAAGAACACCGTGTACCTGCAG
ATGAACTCCCTCAAGTCGGAGGACACCGCTGTCTACTACTGCGCCTCCAGGGGCATCGAGGT
AGAGGAGTTCGGTGCTTGGGGCCAAGGCACCCAGGTGACGGTCTCCTCCGGCGGTGGAGGT
AGCCAGGTCCAGCTGGAGGAGAGTGGCGGCGGCTCCGTGCAGGCCGGCGGTTCGCTGCGCC
TGTCCTGTGCCTACACCTACTCCACGTACTCAAACTACTACATGGGCTGGTTCCGGGAGGCC
CCAGGCAAGGCCCGCACCTCCGTGGCCATCATCAGCTCCGACACCACCATCACTTACAAGGA
CGCCGTGAAAGGTCGTTTCACCATCTCCAAGGACAACGCGAAGAACACCCTGTACCTGCAG
ATGAATTCCCTGAAGCCCGAAGACTCGGCTATGTATAGGTGTGCTGCTTGGACCAGCGATTG
GTCTGTGGCTTATTGGGGCCAGGGCACCCAGGTCACAGTGAGCTCTACATCAACTACAACCC
CCGCCCCGCGCCCCCCAACCCCGGCTCCGACTATCGCTTCCCAGCCATTGTCTCTCCGCCCTG
AAGCTTGTAGACCTGCAGCCGGCGGCGCCGTCCATACTCGCGGTTTGGACTTCGCCTGCGAC
ATCTATATCTGGGCGCCCCTGGCCGGTACCTGCGGGGTGCTGCTGCTGAGTCTGGTCATCAC
CCTTTACTGTAAGCGTGGCCGCAAGAAGCTGTTGTACATCTTCAAGCAGCCCTTCATGCGTC
CGGTGCAGACGACCCAGGAGGAAGACGGATGCTCTTGCCGATTCCCTGAGGAAGAGGAGGG
CGGGTGTGAACTCAGAGTAAAATTTAGCCGCTCGGCTGACGCACCCGCCTACCAGCAGGGA
CAGAACCAGCTGTACAACGAGCTCAACCTGGGCCGCCGCGAAGAGTACGATGTTTTGGATA
AACGCCGCGGTCGAGACCCGGAGATGGGAGGTAAGCCCAGGCGCAAAAACCCTCAGGAGG
GCCTGTACAACGAGCTACAGAAAGACAAGATGGCCGAGGCGTATTCCGAGATCGGTATGAA
GGGCGAGCGGCGCAGAGGGAAAGGCCACGACGGCCTTTATCAGGGCCTCTCCACTGCCACC
AAGGATACTTACGACGCACTTCACATGCAGGCCCTGCCCCCGCGTGGGAGCGGGGCTACCA
ACTTTAGCCTGCTGAAGCAGGCGGGAGATGTGGAGGAGAATCCAGGGCCCATGCGCATCTC
TAAACCTCATTTGCGCTCGATCTCGATTCAGTGCTACCTGTGCCTGCTACTCAACTCCCACTT
TCTGACCGAAGCAGGCATCCATGTTTTCATCTTAGGGTGCTTTAGCGCCGGGCTACCCAAGA
CTGAGGCCAACTGGGTCAACGTGATTTCCGACCTTAAGAAGATTGAGGACCTGATCCAGTCG
ATGCACATTGACGCCACTCTGTACACGGAGTCCGATGTGCACCCCAGCTGTAAGGTGACGGC
TATGAAGTGCTTTCTGCTGGAATTGCAGGTGATTTCCCTGGAGTCTGGAGACGCGTCAATCC
ACGACACGGTAGAGAACCTGATCATCCTGGCGAACAACTCCCTCTCGAGCAATGGCAACGT
GACTGAGAGCGGGTGTAAGGAGTGCGAGGAGCTCGAGGAGAAGAATATCAAGGAGTTCCTG
CAATCCTTCGTCCACATCGTGCAGATGTTTATTAATACTAGC
SEQ ID NO: 16 (Anti-BCMA CD28 CAR armored with soluble IL-15 nucleic acid sequence)
ATGGCTTTGCCGGTGACCGCTCTGCTGCTGCCCCTGGCTTTGCTGCTTCACGCCGCTCGCCCT
GCCGTGCAACTCGTGGAATCTGGCGGCGGACTGGTCCAGGCGGGTGATTCTCTCCGGTTGAC
ATGCACTGCTTCCGGGAGGGCGTTCTCCACCTATTTCATGGCGTGGTTCCGCCAGGCGCCGG
GCAAGGAACGCGAGTTCGTGGCGGGCATCGCGTGGTCTGGGGGTTCGACTGCCTACGCGGA
CAGTGTCAAGGGACGGTTCACCATCAGCCGCGACAACGCGAAGAACACGGTATACCTGCAG
ATGAATAGCCTGAAATCCGAAGATACTGCAGTGTATTACTGTGCCTCCCGCGGTATCGAGGT
GGAGGAGTTCGGCGCCTGGGGCCAGGGCACCCAGGTCACCGTGTCGTCCGGCGGCGGTGGC
TCCCAAGTGCAGTTGGAAGAGAGCGGCGGGGGCTCCGTACAGGCTGGGGGCTCCCTTCGCC
TGAGCTGCGCCTACACCTACTCTACCTACAGCAACTACTACATGGGTTGGTTCAGAGAGGCT
CCCGGGAAGGCGCGCACTTCCGTGGCCATCATCTCTTCCGACACGACTATCACCTACAAGGA
CGCTGTGAAGGGAAGATTCACGATCTCAAAAGACAATGCCAAGAACACTCTCTACCTCCAG
ATGAACTCCCTGAAGCCTGAAGACAGCGCAATGTATAGGTGTGCCGCTTGGACGAGCGATT
GGTCTGTCGCATATTGGGGCCAGGGGACCCAGGTGACAGTGTCCTCGACGAGCACCACCAC
ACCTGCTCCTAGGCCCCCAACTCCGGCGCCCACCATTGCTTCACAGCCACTGTCTCTGCGCCC
GGAGGCCTGCCGACCGGCCGCTGGAGGCGCTGTGCATACACGTGGTTTGGATTTCGCCTGTG
ACATCTACATCTGGGCCCCCCTGGCCGGGACCTGCGGGGTGCTGCTGCTTTCGCTGGTGATC
ACCCTATACTGTCGCTCCAAGCGCAGTCGCCTACTTCACAGTGATTACATGAACATGACTCC
CCGCCGTCCCGGCCCTACCCGCAAGCACTACCAGCCCTATGCCCCCCCGCGTGACTTCGCTG
CTTACCGGAGCCGCGTCAAATTTTCACGCAGTGCGGACGCGCCTGCCTATCAGCAGGGACAG
AACCAGCTTTACAACGAGCTCAACCTGGGCCGGCGCGAGGAGTACGACGTGCTGGACAAGC
GCCGTGGACGTGATCCGGAGATGGGCGGAAAACCTCGGCGCAAAAATCCTCAGGAGGGCCT
TTACAACGAGCTTCAGAAGGACAAAATGGCCGAGGCTTACTCGGAGATCGGTATGAAGGGC
GAGCGCCGTCGCGGCAAAGGGCACGACGGCCTGTACCAGGGATTATCGACTGCTACCAAGG
ATACATACGACGCGCTCCACATGCAGGCCCTGCCTCCCCGTGGCTCCGGTGCAACCAACTTC
TCCCTCCTCAAGCAGGCCGGTGACGTGGAGGAGAATCCAGGCCCCATGCGCATCTCCAAGCC
GCACCTGAGGTCCATTTCCATACAATGTTACCTGTGCCTGTTGCTCAACAGCCACTTTCTGAC
CGAGGCCGGCATCCACGTGTTCATCCTGGGTTGCTTTTCGGCCGGCCTGCCGAAGACCGAGG
CTAACTGGGTTAACGTGATCTCTGATCTAAAGAAGATTGAGGACCTGATCCAGTCCATGCAT
ATTGACGCCACCCTGTACACGGAGAGTGACGTGCACCCCTCTTGTAAGGTGACCGCCATGAA
GTGCTTTCTGCTGGAGCTGCAGGTCATCAGCTTGGAGTCTGGGGACGCATCCATTCATGACA
CCGTGGAGAACCTGATTATCCTGGCCAACAACTCTCTGTCCTCAAATGGCAACGTCACCGAG
AGCGGCTGTAAGGAATGCGAGGAGCTGGAGGAGAAGAACATCAAGGAGTTCCTGCAGTCCT
TCGTCCACATCGTCCAGATGTTTATTAACACGTCT
SEQ ID NO: 17 (Anti-BCMA4-1BB CAR armored with membrane-bound IL-18 nucleic
acid sequence)
ATGGCCCTGCCCGTGACCGCCCTGCTGCTGCCCCTGGCCCTGCTGCTGCACGCCGCCAGG
CCCGCCGTGCAGCTGGTGGAGAGCGGCGGCGGCCTGGTGCAGGCCGGCGACAGCCTGAGG
CTGACCTGCACCGCCAGCGGCAGGGCCTTCAGCACCTACTTCATGGCCTGGTTCAGGCAG
GCCCCCGGCAAGGAGAGGGAGTTCGTGGCCGGCATCGCCTGGAGCGGCGGCAGCACCGCC
TACGCCGACAGCGTGAAGGGCAGGTTCACCATCAGCAGGGACAACGCCAAGAACACCGTG
TACCTGCAGATGAACAGCCTGAAGAGCGAGGACACCGCCGTGTACTACTGCGCCAGCAGG
GGCATCGAGGTGGAGGAGTTCGGCGCCTGGGGCCAGGGCACCCAGGTGACCGTGAGCAGC
GGCGGCGGCGGCAGCCAGGTGCAGCTGGAGGAGAGCGGCGGCGGCAGCGTGCAGGCCGGC
GGCAGCCTGAGGCTGAGCTGCGCCTACACCTACAGCACCTACAGCAACTACTACATGGGC
TGGTTCAGGGAGGCCCCCGGCAAGGCCAGGACCAGCGTGGCCATCATCAGCAGCGACACC
ACCATCACCTACAAGGACGCCGTGAAGGGCAGGTTCACCATCAGCAAGGACAACGCCAAG
AACACCCTGTACCTGCAGATGAACAGCCTGAAGCCCGAGGACAGCGCCATGTACAGGTGC
GCCGCCTGGACCAGCGACTGGAGCGTGGCCTACTGGGGCCAGGGCACCCAGGTGACCGTG
AGCAGCACCAGCACCACCACCCCCGCCCCCAGGCCCCCCACCCCCGCCCCCACCATCGCC
AGCCAGCCCCTGAGCCTGAGGCCCGAGGCCTGCAGGCCCGCCGCCGGCGGCGCCGTGCAC
ACCAGGGGCCTGGACTTCGCCTGCGACATCTACATCTGGGCCCCCCTGGCCGGCACCTGC
GGCGTGCTGCTGCTGAGCCTGGTGATCACCCTGTACTGCAAGAGGGGCAGGAAGAAGCTG
CTGTACATCTTCAAGCAGCCCTTCATGAGGCCCGTGCAGACCACCCAGGAGGAGGACGGC
TGCAGCTGCAGGTTCCCCGAGGAGGAGGAGGGCGGCTGCGAGCTGAGGGTGAAGTTCAGC
AGGAGCGCCGACGCCCCCGCCTACCAGCAGGGCCAGAACCAGCTGTACAACGAGCTGAAC
CTGGGCAGGAGGGAGGAGTACGACGTGCTGGACAAGAGGAGGGGCAGGGACCCCGAGATG
GGCGGCAAGCCCAGGAGGAAGAACCCCCAGGAGGGCCTGTACAACGAGCTGCAGAAGGAC
AAGATGGCCGAGGCCTACAGCGAGATCGGCATGAAGGGCGAGAGGAGGAGGGGCAAGGGC
CACGACGGCCTGTACCAGGGCCTGAGCACCGCCACCAAGGACACCTACGACGCCCTGCAC
ATGCAGGCCCTGCCCCCCAGGGGCAGCGGCGCCACCAACTTCAGCCTGCTGAAGCAGGCC
GGCGACGTGGAGGAGAACCCCGGCCCCATGAGGATCAGCAAGCCCCACCTGAGGAGCATC
AGCATCCAGTGCTACCTGTGCCTGCTGCTGAACAGCCACTTCCTGACCGAGGCCGGCATC
CACGTGTTCATCCTGGGCTGCTTCAGCGCCGGCCTGCCCAAGACCGAGGCCTACTTCGGC
AAGCTGGAGAGCAAGCTGAGCGTGATCAGGAACCTGAACGACCAGGTGCTGTTCATCGAC
CAGGGCAACAGGCCCCTGTTCGAGGACATGACCGACAGCGACTGCAGGGACAACGCCCCC
AGGACCATCTTCATCATCAGCATGTACAAGGACAGCCAGCCCAGGGGCATGGCCGTGACC
ATCAGCGTGAAGTGCGAGAAGATCAGCACCCTGAGCTGCGAGAACAAGATCATCAGCTTC
AAGGAGATGAACCCCCCCGACAACATCAAGGACACCAAGAGCGACATCATCTTCTTCCAG
AGGAGCGTGCCCGGCCACGACAACAAGATGCAGTTCGAGAGCAGCAGCTACGAGGGCTAC
TTCCTGGCCTGCGAGAAGGAGAGGGACCTGTTCAAGCTGATCCTGAAGAAGGAGGACGAG
CTGGGCGACAGGAGCATCATGTTCACCGTGCAGAACGAGGACCCCACCAACGGCCCCAAG
ATCCCCAGCATCGCCACCGGCATGGTGGGCGCCCTGCTGCTGCTGCTGGTGGTGGCCCTG
GGCATCGGCCTGTTCATGAGGAGG
SEQ ID NO: 18 (Anti-CD19 4-1BB CAR nucleic acid sequence)
ATGGCCCTGCCCGTGACCGCCCTGCTGCTGCCCCTGGCCCTGCTGCTGCACGCCGCCAGG
CCCGACATCCAGATGACCCAGACCACCAGCAGCCTGAGCGCCAGCCTGGGCGACAGGGTG
ACCATCAGCTGCAGGGCCAGCCAGGACATCAGCAAGTACCTGAACTGGTACCAGCAGAAG
CCCGACGGCACCGTGAAGCTGCTGATCTACCACACCAGCAGGCTGCACAGCGGCGTGCCC
AGCAGGTTCAGCGGCAGCGGCAGCGGCACCGACTACAGCCTGACCATCAGCAACCTGGAG
CAGGAGGACATCGCCACCTACTTCTGCCAGCAGGGCAACACCCTGCCCTACACCTTCGGC
GGCGGCACCAAGCTGGAGATCACCGGCGGCGGCGGCAGCGGCGGCGGCGGCAGCGGCGGC
GGCGGCAGCGAGGTGAAGCTGCAGGAGAGCGGCCCCGGCCTGGTGGCCCCCAGCCAGAGC
CTGAGCGTGACCTGCACCGTGAGCGGCGTGAGCCTGCCCGACTACGGCGTGAGCTGGATC
AGGCAGCCCCCCAGGAAGGGCCTGGAGTGGCTGGGCGTGATCTGGGGCAGCGAGACCACC
TACTACAACAGCGCCCTGAAGAGCAGGCTGACCATCATCAAGGACAACAGCAAGAGCCAG
GTGTTCCTGAAGATGAACAGCCTGCAGACCGACGACACCGCCATCTACTACTGCGCCAAG
CACTACTACTACGGCGGCAGCTACGCCATGGACTACTGGGGCCAGGGCACCAGCGTGACC
GTGAGCAGCACCACCACCCCCGCCCCCAGGCCCCCCACCCCCGCCCCCACCATCGCCAGC
CAGCCCCTGAGCCTGAGGCCCGAGGCCTGCAGGCCCGCCGCCGGCGGCGCCGTGCACACC
AGGGGCCTGGACTTCGCCTGCGACATCTACATCTGGGCCCCCCTGGCCGGCACCTGCGGC
GTGCTGCTGCTGAGCCTGGTGATCACCCTGTACTGCAAGAGGGGCAGGAAGAAGCTGCTG
TACATCTTCAAGCAGCCCTTCATGAGGCCCGTGCAGACCACCCAGGAGGAGGACGGCTGC
AGCTGCAGGTTCCCCGAGGAGGAGGAGGGCGGCTGCGAGCTGAGGGTGAAGTTCAGCAGG
AGCGCCGACGCCCCCGCCTACAAGCAGGGCCAGAACCAGCTGTACAACGAGCTGAACCTG
GGCAGGAGGGAGGAGTACGACGTGCTGGACAAGAGGAGGGGCAGGGACCCCGAGATGGGC
GGCAAGCCCAGGAGGAAGAACCCCCAGGAGGGCCTGTACAACGAGCTGCAGAAGGACAAG
ATGGCCGAGGCCTACAGCGAGATCGGCATGAAGGGCGAGAGGAGGAGGGGCAAGGGCCAC
GACGGCCTGTACCAGGGCCTGAGCACCGCCACCAAGGACACCTACGACGCCCTGCACATG
CAGGCCCTGCCCCCCAGG
SEQ ID NO: 19 (Anti-CD19 4-1BB CAR armored with soluble IL-18 nucleic acid sequence)
ATGGCCCTGCCCGTGACCGCCCTGCTGCTGCCCCTGGCCCTGCTGCTGCACGCCGCCAGG
CCCGACATCCAGATGACCCAGACCACCAGCAGCCTGAGCGCCAGCCTGGGCGACAGGGTG
ACCATCAGCTGCAGGGCCAGCCAGGACATCAGCAAGTACCTGAACTGGTACCAGCAGAAG
CCCGACGGCACCGTGAAGCTGCTGATCTACCACACCAGCAGGCTGCACAGCGGCGTGCCC
AGCAGGTTCAGCGGCAGCGGCAGCGGCACCGACTACAGCCTGACCATCAGCAACCTGGAG
CAGGAGGACATCGCCACCTACTTCTGCCAGCAGGGCAACACCCTGCCCTACACCTTCGGC
GGCGGCACCAAGCTGGAGATCACCGGCGGCGGCGGCAGCGGCGGCGGCGGCAGCGGCGGC
GGCGGCAGCGAGGTGAAGCTGCAGGAGAGCGGCCCCGGCCTGGTGGCCCCCAGCCAGAGC
CTGAGCGTGACCTGCACCGTGAGCGGCGTGAGCCTGCCCGACTACGGCGTGAGCTGGATC
AGGCAGCCCCCCAGGAAGGGCCTGGAGTGGCTGGGCGTGATCTGGGGCAGCGAGACCACC
TACTACAACAGCGCCCTGAAGAGCAGGCTGACCATCATCAAGGACAACAGCAAGAGCCAG
GTGTTCCTGAAGATGAACAGCCTGCAGACCGACGACACCGCCATCTACTACTGCGCCAAG
CACTACTACTACGGCGGCAGCTACGCCATGGACTACTGGGGCCAGGGCACCAGCGTGACC
GTGAGCAGCACCACCACCCCCGCCCCCAGGCCCCCCACCCCCGCCCCCACCATCGCCAGC
CAGCCCCTGAGCCTGAGGCCCGAGGCCTGCAGGCCCGCCGCCGGCGGCGCCGTGCACACC
AGGGGCCTGGACTTCGCCTGCGACATCTACATCTGGGCCCCCCTGGCCGGCACCTGCGGC
GTGCTGCTGCTGAGCCTGGTGATCACCCTGTACTGCAAGAGGGGCAGGAAGAAGCTGCTG
TACATCTTCAAGCAGCCCTTCATGAGGCCCGTGCAGACCACCCAGGAGGAGGACGGCTGC
AGCTGCAGGTTCCCCGAGGAGGAGGAGGGCGGCTGCGAGCTGAGGGTGAAGTTCAGCAGG
AGCGCCGACGCCCCCGCCTACAAGCAGGGCCAGAACCAGCTGTACAACGAGCTGAACCTG
GGCAGGAGGGAGGAGTACGACGTGCTGGACAAGAGGAGGGGCAGGGACCCCGAGATGGGC
GGCAAGCCCAGGAGGAAGAACCCCCAGGAGGGCCTGTACAACGAGCTGCAGAAGGACAAG
ATGGCCGAGGCCTACAGCGAGATCGGCATGAAGGGCGAGAGGAGGAGGGGCAAGGGCCAC
GACGGCCTGTACCAGGGCCTGAGCACCGCCACCAAGGACACCTACGACGCCCTGCACATG
CAGGCCCTGCCCCCCAGGGGCAGCGGCGCCACCAACTTCAGCCTGCTGAAGCAGGCCGGC
GACGTGGAGGAGAACCCCGGCCCCATGAGGATCAGCAAGCCCCACCTGAGGAGCATCAGC
ATCCAGTGCTACCTGTGCCTGCTGCTGAACAGCCACTTCCTGACCGAGGCCGGCATCCAC
GTGTTCATCCTGGGCTGCTTCAGCGCCGGCCTGCCCAAGACCGAGGCCTACTTCGGCAAG
CTGGAGAGCAAGCTGAGCGTGATCAGGAACCTGAACGACCAGGTGCTGTTCATCGACCAG
GGCAACAGGCCCCTGTTCGAGGACATGACCGACAGCGACTGCAGGGACAACGCCCCCAGG
ACCATCTTCATCATCAGCATGTACAAGGACAGCCAGCCCAGGGGCATGGCCGTGACCATC
AGCGTGAAGTGCGAGAAGATCAGCACCCTGAGCTGCGAGAACAAGATCATCAGCTTCAAG
GAGATGAACCCCCCCGACAACATCAAGGACACCAAGAGCGACATCATCTTCTTCCAGAGG
AGCGTGCCCGGCCACGACAACAAGATGCAGTTCGAGAGCAGCAGCTACGAGGGCTACTTC
CTGGCCTGCGAGAAGGAGAGGGACCTGTTCAAGCTGATCCTGAAGAAGGAGGACGAGCTG
GGCGACAGGAGCATCATGTTCACCGTGCAGAACGAGGAC
SEQ ID NO: 20 (Anti-CD19 4-1BB CAR armored with membrane-bound IL-18 nucleic
acid sequence)
ATGGCCCTGCCCGTGACCGCCCTGCTGCTGCCCCTGGCCCTGCTGCTGCACGCCGCCAGG
CCCGACATCCAGATGACCCAGACCACCAGCAGCCTGAGCGCCAGCCTGGGCGACAGGGTG
ACCATCAGCTGCAGGGCCAGCCAGGACATCAGCAAGTACCTGAACTGGTACCAGCAGAAG
CCCGACGGCACCGTGAAGCTGCTGATCTACCACACCAGCAGGCTGCACAGCGGCGTGCCC
AGCAGGTTCAGCGGCAGCGGCAGCGGCACCGACTACAGCCTGACCATCAGCAACCTGGAG
CAGGAGGACATCGCCACCTACTTCTGCCAGCAGGGCAACACCCTGCCCTACACCTTCGGC
GGCGGCACCAAGCTGGAGATCACCGGCGGCGGCGGCAGCGGCGGCGGCGGCAGCGGCGGC
GGCGGCAGCGAGGTGAAGCTGCAGGAGAGCGGCCCCGGCCTGGTGGCCCCCAGCCAGAGC
CTGAGCGTGACCTGCACCGTGAGCGGCGTGAGCCTGCCCGACTACGGCGTGAGCTGGATC
AGGCAGCCCCCCAGGAAGGGCCTGGAGTGGCTGGGCGTGATCTGGGGCAGCGAGACCACC
TACTACAACAGCGCCCTGAAGAGCAGGCTGACCATCATCAAGGACAACAGCAAGAGCCAG
GTGTTCCTGAAGATGAACAGCCTGCAGACCGACGACACCGCCATCTACTACTGCGCCAAG
CACTACTACTACGGCGGCAGCTACGCCATGGACTACTGGGGCCAGGGCACCAGCGTGACC
GTGAGCAGCACCACCACCCCCGCCCCCAGGCCCCCCACCCCCGCCCCCACCATCGCCAGC
CAGCCCCTGAGCCTGAGGCCCGAGGCCTGCAGGCCCGCCGCCGGCGGCGCCGTGCACACC
AGGGGCCTGGACTTCGCCTGCGACATCTACATCTGGGCCCCCCTGGCCGGCACCTGCGGC
GTGCTGCTGCTGAGCCTGGTGATCACCCTGTACTGCAAGAGGGGCAGGAAGAAGCTGCTG
TACATCTTCAAGCAGCCCTTCATGAGGCCCGTGCAGACCACCCAGGAGGAGGACGGCTGC
AGCTGCAGGTTCCCCGAGGAGGAGGAGGGCGGCTGCGAGCTGAGGGTGAAGTTCAGCAGG
AGCGCCGACGCCCCCGCCTACAAGCAGGGCCAGAACCAGCTGTACAACGAGCTGAACCTG
GGCAGGAGGGAGGAGTACGACGTGCTGGACAAGAGGAGGGGCAGGGACCCCGAGATGGGC
GGCAAGCCCAGGAGGAAGAACCCCCAGGAGGGCCTGTACAACGAGCTGCAGAAGGACAAG
ATGGCCGAGGCCTACAGCGAGATCGGCATGAAGGGCGAGAGGAGGAGGGGCAAGGGCCAC
GACGGCCTGTACCAGGGCCTGAGCACCGCCACCAAGGACACCTACGACGCCCTGCACATG
CAGGCCCTGCCCCCCAGGGGCAGCGGCGCCACCAACTTCAGCCTGCTGAAGCAGGCCGGC
GACGTGGAGGAGAACCCCGGCCCCATGAGGATCAGCAAGCCCCACCTGAGGAGCATCAGC
ATCCAGTGCTACCTGTGCCTGCTGCTGAACAGCCACTTCCTGACCGAGGCCGGCATCCAC
GTGTTCATCCTGGGCTGCTTCAGCGCCGGCCTGCCCAAGACCGAGGCCTACTTCGGCAAG
CTGGAGAGCAAGCTGAGCGTGATCAGGAACCTGAACGACCAGGTGCTGTTCATCGACCAG
GGCAACAGGCCCCTGTTCGAGGACATGACCGACAGCGACTGCAGGGACAACGCCCCCAGG
ACCATCTTCATCATCAGCATGTACAAGGACAGCCAGCCCAGGGGCATGGCCGTGACCATC
AGCGTGAAGTGCGAGAAGATCAGCACCCTGAGCTGCGAGAACAAGATCATCAGCTTCAAG
GAGATGAACCCCCCCGACAACATCAAGGACACCAAGAGCGACATCATCTTCTTCCAGAGG
AGCGTGCCCGGCCACGACAACAAGATGCAGTTCGAGAGCAGCAGCTACGAGGGCTACTTC
CTGGCCTGCGAGAAGGAGAGGGACCTGTTCAAGCTGATCCTGAAGAAGGAGGACGAGCTG
GGCGACAGGAGCATCATGTTCACCGTGCAGAACGAGGACCCCACCAACGGCCCCAAGATC
CCCAGCATCGCCACCGGCATGGTGGGCGCCCTGCTGCTGCTGCTGGTGGTGGCCCTGGGC
ATCGGCCTGTTCATGAGGAGG
SEQ ID NO: 21 (Anti-GPC3 4-1BB CAR nucleic acid sequence)
ATGGCCCTGCCCGTGACCGCCCTGCTGCTGCCCCTGGCCCTGCTGCTGCACGCCGCCAGG
CCCGACGTGGTGATGACCCAGAGCCCCCTGAGCCTGCCCGTGACCCCCGGCGAGCCCGCC
AGCATCAGCTGCAGGAGCAGCCAGAGCCTGGTGCACAGCAACGCCAACACCTACCTGCAC
TGGTACCTGCAGAAGCCCGGCCAGAGCCCCCAGCTGCTGATCTACAAGGTGAGCAACAGG
TTCAGCGGCGTGCCCGACAGGTTCAGCGGCAGCGGCAGCGGCACCGACTTCACCCTGAAG
ATCAGCAGGGTGGAGGCCGAGGACGTGGGCGTGTACTACTGCAGCCAGAACACCCACGTG
CCCCCCACCTTCGGCCAGGGCACCAAGCTGGAGATCAAGAGGGGCGGCGGCGGCAGCGGC
GGCGGCGGCAGCGGCGGCGGCGGCAGCCAGGTGCAGCTGGTGCAGAGCGGCGCCGAGGTG
AAGAAGCCCGGCGCCAGCGTGAAGGTGAGCTGCAAGGCCAGCGGCTACACCTTCACCGAC
TACGAGATGCACTGGGTGAGGCAGGCCCCCGGCCAGGGCCTGGAGTGGATGGGCGCCCTG
GACCCCAAGACCGGCGACACCGCCTACAGCCAGAAGTTCAAGGGCAGGGTGACCCTGACC
GCCGACGAGAGCACCAGCACCGCCTACATGGAGCTGAGCAGCCTGAGGAGCGAGGACACC
GCCGTGTACTACTGCACCAGGTTCTACAGCTACACCTACTGGGGCCAGGGCACCCTGGTG
ACCGTGAGCAGCACCACCACCCCCGCCCCCAGGCCCCCCACCCCCGCCCCCACCATCGCC
AGCCAGCCCCTGAGCCTGAGGCCCGAGGCCTGCAGGCCCGCCGCCGGCGGCGCCGTGCAC
ACCAGGGGCCTGGACTTCGCCTGCGACATCTACATCTGGGCCCCCCTGGCCGGCACCTGC
GGCGTGCTGCTGCTGAGCCTGGTGATCACCCTGTACTGCAAGAGGGGCAGGAAGAAGCTG
CTGTACATCTTCAAGCAGCCCTTCATGAGGCCCGTGCAGACCACCCAGGAGGAGGACGGC
TGCAGCTGCAGGTTCCCCGAGGAGGAGGAGGGCGGCTGCGAGCTGAGGGTGAAGTTCAGC
AGGAGCGCCGACGCCCCCGCCTACAAGCAGGGCCAGAACCAGCTGTACAACGAGCTGAAC
CTGGGCAGGAGGGAGGAGTACGACGTGCTGGACAAGAGGAGGGGCAGGGACCCCGAGATG
GGCGGCAAGCCCAGGAGGAAGAACCCCCAGGAGGGCCTGTACAACGAGCTGCAGAAGGAC
AAGATGGCCGAGGCCTACAGCGAGATCGGCATGAAGGGCGAGAGGAGGAGGGGCAAGGGC
CACGACGGCCTGTACCAGGGCCTGAGCACCGCCACCAAGGACACCTACGACGCCCTGCAC
ATGCAGGCCCTGCCCCCCAGG
SEQ ID NO: 22 (Anti-GPC3 4-1BB CAR armored with soluble IL-18 nucleic acid sequence)
ATGGCCCTGCCCGTGACCGCCCTGCTGCTGCCCCTGGCCCTGCTGCTGCACGCCGCCAGG
CCCGACGTGGTGATGACCCAGAGCCCCCTGAGCCTGCCCGTGACCCCCGGCGAGCCCGCC
AGCATCAGCTGCAGGAGCAGCCAGAGCCTGGTGCACAGCAACGCCAACACCTACCTGCAC
TGGTACCTGCAGAAGCCCGGCCAGAGCCCCCAGCTGCTGATCTACAAGGTGAGCAACAGG
TTCAGCGGCGTGCCCGACAGGTTCAGCGGCAGCGGCAGCGGCACCGACTTCACCCTGAAG
ATCAGCAGGGTGGAGGCCGAGGACGTGGGCGTGTACTACTGCAGCCAGAACACCCACGTG
CCCCCCACCTTCGGCCAGGGCACCAAGCTGGAGATCAAGAGGGGCGGCGGCGGCAGCGGC
GGCGGCGGCAGCGGCGGCGGCGGCAGCCAGGTGCAGCTGGTGCAGAGCGGCGCCGAGGTG
AAGAAGCCCGGCGCCAGCGTGAAGGTGAGCTGCAAGGCCAGCGGCTACACCTTCACCGAC
TACGAGATGCACTGGGTGAGGCAGGCCCCCGGCCAGGGCCTGGAGTGGATGGGCGCCCTG
GACCCCAAGACCGGCGACACCGCCTACAGCCAGAAGTTCAAGGGCAGGGTGACCCTGACC
GCCGACGAGAGCACCAGCACCGCCTACATGGAGCTGAGCAGCCTGAGGAGCGAGGACACC
GCCGTGTACTACTGCACCAGGTTCTACAGCTACACCTACTGGGGCCAGGGCACCCTGGTG
ACCGTGAGCAGCACCACCACCCCCGCCCCCAGGCCCCCCACCCCCGCCCCCACCATCGCC
AGCCAGCCCCTGAGCCTGAGGCCCGAGGCCTGCAGGCCCGCCGCCGGCGGCGCCGTGCAC
ACCAGGGGCCTGGACTTCGCCTGCGACATCTACATCTGGGCCCCCCTGGCCGGCACCTGC
GGCGTGCTGCTGCTGAGCCTGGTGATCACCCTGTACTGCAAGAGGGGCAGGAAGAAGCTG
CTGTACATCTTCAAGCAGCCCTTCATGAGGCCCGTGCAGACCACCCAGGAGGAGGACGGC
TGCAGCTGCAGGTTCCCCGAGGAGGAGGAGGGCGGCTGCGAGCTGAGGGTGAAGTTCAGC
AGGAGCGCCGACGCCCCCGCCTACAAGCAGGGCCAGAACCAGCTGTACAACGAGCTGAAC
CTGGGCAGGAGGGAGGAGTACGACGTGCTGGACAAGAGGAGGGGCAGGGACCCCGAGATG
GGCGGCAAGCCCAGGAGGAAGAACCCCCAGGAGGGCCTGTACAACGAGCTGCAGAAGGAC
AAGATGGCCGAGGCCTACAGCGAGATCGGCATGAAGGGCGAGAGGAGGAGGGGCAAGGGC
CACGACGGCCTGTACCAGGGCCTGAGCACCGCCACCAAGGACACCTACGACGCCCTGCAC
ATGCAGGCCCTGCCCCCCAGGGGCAGCGGCGCCACCAACTTCAGCCTGCTGAAGCAGGCC
GGCGACGTGGAGGAGAACCCCGGCCCCATGAGGATCAGCAAGCCCCACCTGAGGAGCATC
AGCATCCAGTGCTACCTGTGCCTGCTGCTGAACAGCCACTTCCTGACCGAGGCCGGCATC
CACGTGTTCATCCTGGGCTGCTTCAGCGCCGGCCTGCCCAAGACCGAGGCCTACTTCGGC
AAGCTGGAGAGCAAGCTGAGCGTGATCAGGAACCTGAACGACCAGGTGCTGTTCATCGAC
CAGGGCAACAGGCCCCTGTTCGAGGACATGACCGACAGCGACTGCAGGGACAACGCCCCC
AGGACCATCTTCATCATCAGCATGTACAAGGACAGCCAGCCCAGGGGCATGGCCGTGACC
ATCAGCGTGAAGTGCGAGAAGATCAGCACCCTGAGCTGCGAGAACAAGATCATCAGCTTC
AAGGAGATGAACCCCCCCGACAACATCAAGGACACCAAGAGCGACATCATCTTCTTCCAG
AGGAGCGTGCCCGGCCACGACAACAAGATGCAGTTCGAGAGCAGCAGCTACGAGGGCTAC
TTCCTGGCCTGCGAGAAGGAGAGGGACCTGTTCAAGCTGATCCTGAAGAAGGAGGACGAG
CTGGGCGACAGGAGCATCATGTTCACCGTGCAGAACGAGGAC

### REFERENCES

1. Hay KA, Turtle CJ. Chimeric Antigen Receptor (CAR) T Cells: Lessons Learned from Targeting of CD19 in B-Cell Malignancies. Drugs. 2017; 77(3):237-245.
2. Boyiadzis, M. M., Dhodapkar, M. V., Brentjens, R. J. et al. Chimeric antigen receptor (CAR) T therapies for the treatment of hematologic malignancies: clinical perspective and significance.j. immunotherapy cancer 2018; 6, 137
3. Ma S, Li X, Wang X, et al. Current Progress in CAR-T Cell Therapy for Solid Tumors. IntJBiol Sci. 2019; 15(12):2548-2560.
4. Kheng N, Shaun OB, et al. CAR T Cell Therapy for Solid Tumors. Annu Rev Med. e 2017 68:1, 139-152
5. Gill S. How close are we to CAR T-cell therapy for AML? Best Pract Res Clin Haematol. 2019 December; 32(4):101104
6. Rotolo R, Leuci V, Donini C, et al. CAR-Based Strategies beyond T Lymphocytes: Integrative Opportunities for Cancer Adoptive Immunotherapy. Int J Mol Sci. 2019;20(11):2839.
7. Yazdanifar, Mahboubeh et al. “γδ T Cells: The Ideal Tool for Cancer Immunotherapy.” Cells vol. 9,5 E1305. 24 May. 2020.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An engineered γδ T cell comprising:
(i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from, a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

2. The engineered γδ T cell of claim 1, wherein the IL-18 receptor comprises IL-18Rα, IL-18Rβ or the combination thereof.

3. The engineered γδ T cell of claim 1, wherein the chimeric cytokine receptor further comprises the exodomain of a cytokine other than IL-18, or an artificial ligand.

4. The engineered γδ T cell of claim 1, wherein the IL-18 is in soluble form or membrane-bound form.

5. The engineered γδ T cell of any one of the preceding claims, wherein the engineered γδ T cell is selected from the group consisting of γδ T cell, δ1 T cell, δ3 T cell, or the combination thereof.

6. The engineered γδ T cell of claim 1, wherein the first nucleic acid further comprises a first regulatory region which comprises a promoter operatively linked to the first nucleic acid sequence.

7. The engineered γδ T cell of claim 1, wherein the second nucleic acid sequence further comprises a second regulatory region operatively linked to the second nucleic acid sequence.

8. The engineered γδ T cell of claim 7, wherein the second regulatory region comprises (i) an inducible promoter, and/or (ii) a promoter and one or more transcription factor binding sites, wherein the transcription factor binding sites bind to transcription factors that are active in activated γδ T cells.

9. The engineered γδ T cell of claim 8, wherein the transcription factor binding sites comprise one or more copies of the transcription factor binding site selected from the group consisting of NF-κB, AP-1, Myc, NR4A, TOX1, TOX2, TOX3, TOX4, STAT1, STAT2, STAT3, STAT4, STAT5, STAT6, or combinations thereof.

10. The engineered γδ T cell of claim 8, wherein the promoter comprises an IFN-β promoter, an IL-2 promoter, an BCL-2 promoter, a GM-CSF promoter, an IL-6 promoter, an IFN-γ promoter, an IL-12 promoter, an IL-4 promoter, an IL-15 promoter, an IL-18 promoter or an IL-21 promoter.

11. The engineered γδ T cell of any one of the preceding claims, wherein the first nucleic acid and the second nucleic acid are comprised in one vector.

12. The engineered γδ T cell of claim 11, wherein the first nucleic acid and the second nucleic acid are transcribed in opposite directions.

13. The engineered γδ T cell of any one of claims 1 to 10, wherein the first nucleic acid and the second nucleic acid are comprised in separate vectors.

14. The engineered γδ T cell of claims 11 or 13, wherein the vector is a virus vector.

15. The engineered γδ T cell of claim 14, wherein the virus vector is a lentivirus vector, retrovirus vector, adenoviral vectors, adeno-associated virus vectors, vaccinia vector, or herpes simplex viral vector.

16. The engineered γδ T cell of any one of the preceding claims, wherein the extracellular antigen recognition domain is selective for a tumor antigen or an infectious disease-associated antigen.

17. The engineered γδ T cell of claim 16, wherein the tumor antigen is selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof.

18. The engineered γδ T cell of any one of the preceding claims, wherein the extracellular antigen recognition domain is multispecific.

19. The engineered γδ T cell of any one of the preceding claims, wherein the CAR is a tandem CAR or dual CAR.

20. The engineered γδ T cell of claim 1 or 19, wherein the CAR targets the same tumor antigen.

21. The engineered γδ T cell of claim 20, wherein the CAR targets different epitopes on the same tumor antigen.

22. The engineered γδ T cell of claim 1 or 19, wherein the CAR targets different tumor antigens.

23. The engineered γδ T cell of any one of claims 20 to 22, wherein the tumor antigen comprises BCMA, CD19 and/or GPC3.

24. The engineered γδ T cell of claim 19, wherein the tandem CAR comprises: more than one antigen-binding portions that target different epitopes on BCMA, CD19 or GPC3, a transmembrane domain, and an intracellular signaling domain.

25. The engineered γδ T cell of any one of the preceding claims, wherein the intracellular signaling domain comprises a primary intracellular signaling domain of an immune effector cell derived from a signal transducing molecule selected from the group consisting of CD3ζ, FcRγ, FcRβ, CD3γ, CD3δ, CD3ε, CD5, CD22, CD79a, CD79b, CD66d and combinations thereof.

26. The engineered γδ T cell of any one of the preceding claims, wherein the intracellular signaling domain comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.

27. The engineered γδ T cell of any one of the preceding claims, wherein the transmembrane domain is from CD4, CD8α, CD28, or ICOS.

28. The engineered γδ T cell of any one of the preceding claims, wherein the nucleic acid sequence that encodes a CAR further comprises a hinge region located between the extracellular antigen recognition domain and the transmembrane domain.

29. The engineered γδ T cell of any one of the preceding claims, wherein both the first nucleic acid and the second nucleic acid have a leading peptide.

30. The engineered γδ T cell of any one of the preceding claims, wherein the engineered γδ T cell comprises a nucleic acid having a nucleotide sequence at least about 80%, 85%, 90%, 91%, 92%, 93%, 94%, 95%, 96%, 97%, 98%, 99% or 100% identical to SEQ ID NO: 14,17,19, 20 or 22.

31. The engineered γδ T cell of claim 30, wherein the engineered γδ T cell comprises a nucleic acid having a nucleotide sequence of any one of SEQ ID NO: 14,17,19, 20 or 22.

32. The engineered γδ T cell of any one of the preceding claims, wherein the engineered γδ T cell is allogeneic.

33. The engineered γδ T cell of any one of the preceding claims, wherein the engineered γδ T cell is autologous.

34. An engineered γδ T cell comprising:
(i) a first nucleic acid, which comprises a first regulatory region operatively linked to a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from, a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R),
wherein the extracellular antigen recognition domain is selective for a tumor antigen selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof;
the intracellular signaling domain comprises a primary intracellular signaling domain of an immune effector cell derived from a signal transducing molecule selected from the group consisting of CD3ζ, FcRγ, FcRβ, CD3γ, CD38, CD3z, CD5, CD22, CD79a, CD79b, CD66d and combinations thereof; and the intracellular signaling domain further comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof;
the transmembrane domain is from CD4, CD8α, CD28, or ICOS; and
optionally, the second nucleic acid sequence further comprises a second regulatory region which is inducible and operatively linked to the second nucleic acid sequence.

35. An engineered γδ T cell comprising:
(i) a first nucleic acid, which comprises a first regulatory region operatively linked to a first nucleic acid sequence that encodes a chimeric antigen receptor (CAR) comprising: more than one tandem antigen recognition portions targeting BCMA, CD19 and/or GPC3; a transmembrane domain selected from CD4, CD8α, CD28, or ICOS; a CD3(intracellular signaling domain; and a CD28 or 4-1BB intracellular co-stimulatory domain; and
(ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R).

36. An engineered γδ T cell comprising:
(i) a chimeric antigen receptor (CAR) comprising an extracellular antigen recognition domain that is selective for a target, a transmembrane domain, and an intracellular signaling domain, and/or
a T cell receptor (TCR) or antigen recognition domain fused to the CD3 chain of a TCR complex, where the TCR complex comprising (a) a TCR chain selected from gamma chain and a delta chain of a T cell receptor, (b) an epsilon chain, a delta chain, and/or a gamma chain of CD3, or (c) a zeta chain of CD3; and
(ii) an exogenous cytokine IL-18 or a functional variant thereof, or a chimeric cytokine receptor comprising the endodomain of the IL-18 receptor (IL-18R).

37. The engineered γδ T cell of claim 36, wherein the extracellular antigen recognition domain is selective for a tumor antigen selected from the group consisting of CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, HER-2 and combinations thereof;
the intracellular signaling domain comprises a primary intracellular signaling domain of an immune effector cell derived from a signal transducing molecule selected from the group consisting of CD3, FcRγ, FcRβ, CD3γ, CD3δ, CD3ε, CD5, CD22, CD79a, CD79b, CD66d and combinations thereof; and/or the intracellular signaling domain comprises an intracellular co-stimulatory domain derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof; and
the transmembrane domain is from CD4, CD8α, CD28, or ICOS.

38. The engineered γδ T cell of claim 36 or 37, wherein the IL-18 receptor comprises IL-18Rα, IL-18R3 or the combination thereof.

39. The engineered γδ T cell of claim 36 or 37, wherein the chimeric cytokine receptor further comprises the exodomain of a cytokine other than IL-18, or an artificial ligand.

40. The engineered γδ T cell of claim 36 or 37, wherein the IL-18 is in soluble form or membrane-bound form.

41. The engineered γδ T cell of any one of claims 36 to 40, wherein the CAR is a tandem CAR targeting BCMA, CD19 and/or GPC3.

42. An engineered γδ T cell comprising:
(i) a tandem chimeric antigen receptor (CAR) comprising more than one antigen recognition portions targeting BCMA, CD19 and/or GPC3, a transmembrane domain, and an intracellular signaling domain; and
(ii) an exogenous cytokine IL-18 or a functional variant thereof, or a chimeric cytokine receptor comprising the endodomain of the IL-18 receptor (IL-18R). wherein the tandem CAR targets the same tumor antigen or different tumor antigens.

43. The engineered γδ T of claim 42, wherein the intracellular signaling domain is CD3ζ, the intracellular signaling domain also comprises an intracellular co-stimulatory domain CD28 or 4-1BB, and the transmembrane domain is from CD4, CD8α, CD28, or ICOS.

44. The engineered γδ T of claim 42 or 43, wherein the IL-18 receptor comprises IL-18Rα, IL-18Rβ or the combination thereof.

45. The engineered γδ T cell of any one of claims 42 to 44, wherein the chimeric cytokine receptor further comprises the exodomain of a cytokine other than IL-18, or an artificial ligand.

46. The engineered γδ T cell of claim 42 or 43, wherein the IL-18 is in soluble form or membrane-bound form.

47. The engineered γδ T cell of claim 42, wherein the engineered γδ T cell comprises a polypeptide having an amino acid sequence at least about 80%, 85%, 90%, 91%, 92%, 93%, 94%, 95%, 96%, 97%, 98%, 99% or 100% identical to SEQ ID NO: 2, 5, 7, 8 or 10.

48. The engineered γδ T cell of claim 47, wherein the engineered γδ T cell comprises a polypeptide having an amino acid sequence of any one of SEQ ID NO: 2, 5, 7, 8 or 10.

49. A pharmaceutical composition, comprising an effective amount of the engineered γδ T cell of any one of the preceding claims and a pharmaceutically acceptable excipient.

50. The pharmaceutical composition of claim 49, wherein the composition comprises a therapeutically effective amount of the engineered γδ T cell for treating a hematological cancer or solid tumor.

51. A method of providing an anti-tumor immunity in a subject comprising administering to the subject an effective amount of the engineered γδ T cell of any one of claims 1 to 48 or the pharmaceutical composition of claim 49 or 50.

52. A method of treating cancer in a subject, the method comprising administering to the subject an effective amount of the engineered γδ T cell of claims 1 to 48 or the pharmaceutical composition of claim 49 or 50, wherein the engineered γδ T cells treat the cancer.

53. A method of delaying or preventing metastasis or recurrence of a cancer in a subject, the method comprising administering to the subject an effective amount of the engineered γδ T cell of claims 1 to 48 or the pharmaceutical composition of claim 49 or 50, wherein the engineered γδ T cells delay or prevent metastasis or recurrence of the cancer.

54. A method of making a chimeric antigen receptor γδ T cell armored with IL-18, which comprises introducing into a γδ T cell:
(i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from, a gamma chain and a delta chain of a T cell receptor, (b) an epsilon chain, a delta chain, and/or a gamma chain of CD3, or (c) a zeta chain of CD3; and
(ii) a second nucleic acid, which comprises a second nucleic acid sequence that encodes an exogenous cytokine IL-18 or a functional variant thereof, or a chimeric cytokine receptor comprising the endodomain of the IL-18 receptor (IL-18R).

55. A kit for making a chimeric antigen receptor γδ T cell armored with IL-18, which comprises:
(a) a container comprising
(1) (i) a first nucleic acid, which comprises a first nucleic acid sequence that encodes a chimeric antigen receptor (CAR) comprising an extracellular antigen recognition domain that is selective for a target, a transmembrane domain, and an intracellular signaling domain, and/or
a first nucleic acid, which comprises a first nucleic acid sequence that encodes a T cell receptor (TCR) or antigen recognition domain fused to the CD3 chain of a TCR complex, where the TCR complex comprising (a) a TCR chain selected from a gamma chain and a delta chain of a T cell receptor, (b) an epsilon chain, a delta chain, and/or a gamma chain of CD3, or (c) a zeta chain of CD3; and
(ii) a second nucleic acid, which comprises a nucleic acid sequence that encodes an exogenous cytokine IL-18 or a functional variant thereof, or a chimeric cytokine receptor comprising the endodomain of the IL-18 receptor (IL-18R);
or
(2) a vector comprising the first and second nucleic acids;
(b) a container comprising γδ T cells; and
(c) instructions for using the kit.

56. Use of the engineered γδ T cell of claims 1 to 48 or the pharmaceutical composition of claim 49 or 50, to treat a cancer or an infectious disease in a subject.
